(19) United States
(12) Patent Application Publication
(10) Pub. No.: US 2006/0264440 A1
(43) Pub. Date: Nov. 23, 2006

(54) Pyrazoles and methods of making and using the same

(75) Inventors: Wen-Cherng Lee, Lexington, MA (US); Lihong Sun, Arlington, MA (US); Feng Shan, Braintree, MA (US); Claudio Chuaqui, Somerville, MA (US); Mark Cornebise, Watertown, MA (US); Timothy W. Pontz, Brighton, MA (US); Mary Beth Carter, Arlington, MA (US); Juswinder Singh, Ashland, MA (US); Paula Ann Boriack-Sjodin, Waltham, MA (US); Leona Ling, Winchester, MA (US); Russell C. Petter, Stow, MA (US)

Correspondence Address: Jonathan P O'Brien;MILLER CANFIELD PADDOCK & STONE, 444 West Michigan Avenue, Kalamazoo, MI, 49007, US

(73) Assignee: BIOGEN IDEC MA INC., Cambridge, MA (US)

(21) Appl. No.: 10/545,179

(22) PCT Filed: Feb. 12, 2004
(86) PCT No.: PCT/US04/04049

§ 371 Date: Apr. 26, 2006

Related U.S. Application Data
(60) Provisional application No. 60/446,777, filed on Feb. 12, 2003.

(51) Int. Cl. A61K 31/522; A61K 31/52; A61K 31/519; A61K 31/517; C07D 473/02; C07D 487/04

(52) U.S. Cl. 514/249; 544/238; 544/235; 544/281; 544/284; 544/353; 544/277; 544/278; 514/252.02; 514/252.04; 514/263.2; 514/260.1; 514/259.1; 514/266.2; 514/266.21

(57) ABSTRACT

The invention is based on the discovery that compounds of formula I possess unexpectedly high affinity for Alk 5 and/or Alk 4, and can be useful as antagonists thereof for preventing and/or treating numerous diseases, including fibrotic disorders. In one embodiment, the invention features a compound of formula I (I).

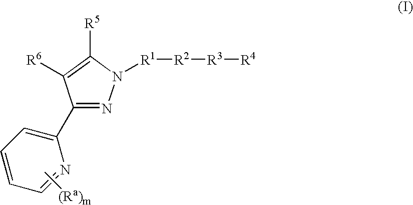

### BACKGROUND OF THE INVENTION

TGFβ (Transforming Growth Factor β) is a member of a large family of dimeric polypeptide growth factors that includes activins, inhibins, bone morphogenetic proteins (BMPs), growth and differentiation factors (GDFs) and mullerian inhibiting substance (MIS). TGFβ exists in three isoforms (TGFβ1, TGFβ2, and TGFβ3) and is present in most cells, along with its receptors. Each isoform is expressed in both a tissue-specific and developmentally regulated fashion. Each TGFβ isoform is synthesized as a precursor protein that is cleaved intracellularly into a C-terminal region (latency associated peptide (LAP)) and an N-terminal region known as mature or active TGFβ. LAP is typically non-covalently associated with mature TGFβ prior to secretion from the cell. The LAP-TGFβ complex cannot bind to the TGFβ receptors and is not biologically active. TGFβ is generally released (and activated) from the complex by a variety of mechanisms including interaction with thrombospondin-1 or plasmin.

Following activation, TGFβ binds at high affinity to the type II receptor (TGFβRII), a constitutively active serine/threonine kinase. The ligand-bound type II receptor phosphorylates the TGFβ type I receptor (Alk 5) in a glycine/serine rich domain, which allows the type I receptor to recruit and phosphorylate downstream signaling molecules, Smad2 or Smad3. See, e.g., Huse, M. et al., Mol. Cell. 8: 671-682 (2001). Phosphorylated Smad2 or Smad3 can then complex with Smad4, and the entire hetero-Smad complex translocates to the nucleus and regulates transcription of various TGFβ-responsive genes. See, e.g., Massagué, J. Ann. Rev. Biochem. Med. 67: 773 (1998).

Activins are also members of the TGFβ superfamily which are distinct from TGFβ in that they are homo- or heterodimers of activin βa or βb. Activins signal in a similar manner to TGFβ, that is, by binding to a constitutive serine-threonine receptor kinase, activin type II receptor (ActRIIB), and activating a type I serine-threonine receptor, Alk 4, to phosphorylate Smad2 or Smad3. The consequent formation of a hetero-Smad complex with Smad4 also results in the activin-induced regulation of gene transcription.

Indeed, TGFβ and related factors such as activin regulate a large array of cellular processes, e.g., cell cycle arrest in epithelial and hematopoietic cells, control of mesenchymal cell proliferation and differentiation, inflammatory cell recruitment, immunosuppression, wound healing, and extracellular matrix production. See, e.g., Massagué, J. Ann. Rev. Cell. Biol. 6: 594-641 (1990); Roberts, A. B. and Sporn M. B. Peptide Growth Factors and Their Receptors, 95: 419-472 Berlin: Springer-Verlag (1990); Roberts, A. B. and Sporn M. B. Growth Factors 8:1-9 (1993); and Alexandrow, M. G., Moses, H. L. Cancer Res. 55: 1452-1457 (1995). Hyperactivity of TGFβ signaling pathway underlies many human disorders (e.g., excess deposition of extracellular matrix, an abnormally high level of inflammatory responses, fibrotic disorders, and progressive cancers). Similarly, activin signaling and overexpression of activin is linked to pathological disorders that involve extracellular matrix accumulation and fibrosis (see, e.g., Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); Sugiyama, M. et al., Gastroenterology 114: 550-558 (1998); Munz, B. et al., EMBO J. 18: 5205-5215 (1999)), inflammatory responses (see, e.g., Rosendahl, A. et al., Am. J. Repir. Cell Mol. Biol. 25: 60-68 (2001)), cachexia or wasting (see Matzuk, M. M. et al., Proc. Nat. Acad. Sci. USA 91: 8817-8821 (1994); Coerver, K. A. et al, Mol. Endocrinol. 10: 534-543 (1996); Cipriano, S. C. et al. Endocrinology 141: 2319-27 (2000)), diseases of or pathological responses in the central nervous system (see Logan, A. et al. Eur. J. Neurosci. 11: 2367-2374 (1999); Logan, A. et al. Exp. Neurol. 159: 504-510 (1999); Masliah, E. et al., Neurochem. Int. 39: 393-400 (2001); De Groot, C. J. A. et al, J. Neuropathol. Exp. Neurol. 58: 174-187 (1999), John, G. R. et al, Nat Med. 8: 1115-21 (2002)) and hypertension (see Dahly, A. J. et al., Am. J. Physiol. Regul. Integr. Comp. Physiol. 283: R757-67 (2002)). Studies have also shown that TGFβ and activin can act synergistically to induce extracellular matrix (see, e.g., Sugiyama, M. et al., Gastroenterology 114: 550-558, (1998)). It is therefore desirable to develop modulators (e.g., antagonists) to signaling pathway components of the TGFβ family to prevent/treat disorders related to the malfunctioning of this signaling pathway.

### SUMMARY OF THE INVENTION

The invention is based on the discovery that compounds of formula (I) are unexpectedly potent antagonists of the TGFβ family type I receptors, Alk5 and/or Alk 4. Thus, compounds of formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

In one aspect, the invention features a compound of formula I:

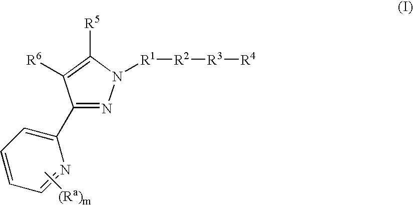

Each R^{a} is independently alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonyl amino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, or heteroaroyl. R¹ is a bond, alkylene, alkenylene, alkynylene, or —(CH₂)ᵣ₁—O—(CH₂)ᵣ₂—, where each of r1 and r2 is independently 2 or 3. R² is cycloalkyl, heterocycloalkyl, cycloalkenyl, heterocycloalkenyl, aryl, heteroaryl, or a bond. R³ is —C(O)—, —C(O)O—, —OC(O)—, —C(O)—N(R^{b})—, —N(R^{b})—C(O)—, —O—C(O)—N(R^{b})—, —N(R^{b})—C(O)—O—, —O—S(O)ₚ—N(R^{b})—, —N(R^{b})—S(O)ₚ—O—, —N(R^{b})—C(O)—R^{c})—, —N(R^{b})—S(O)ₚ—N(R^{b})—, —C(O)—N(R^{b})—S(O)ₚ—, —S(O)ₚ—N(R^{b})—C(O)—, —S(O)ₚ—N(R^{b})—, —N(R^{b})—S(O)ₚ—, —N(R^{b})—, —S(O)ₚ—, —O—, —S—, or —(C(R^{b})(R^{c}))_{q}—, or a bond. Each of R^{b} and R^{c} is independently hydrogen, hydroxy, alkyl, aryl, aralkyl, heterocycloalkyl, heteroaryl, or heteroaralkyl. p is 1 or 2; and q is 1-4. R⁴ is hydrogen, alkyl, alkenyl, alkynyl, cycloalkyl, (cycloalkyl)alkyl, heterocycloalkyl, (heterocycloalkyl)alkyl, cycloalkenyl, (cycloalkenyl)alkyl, heterocycloalkenyl, (heterocycloalkenyl)alkyl, aryl, aralkyl, heteroaryl, or heteroaralkyl. R⁵ is hydrogen, unsubstituted alkyl, halo-substituted alkyl, alkoxy, alkylsulfinyl, ammo, alkenyl, alkynyl, cycloalkyl, cycloalkoxy, cycloalkylsulfinyl, heterocycloalkyl, heterocycloalkoxy, heterocycloalkylsulfinyl, aryl, aryloxy, arylsulfinyl, heteroaryl, heteroaryloxy, or heteroarylsulfiyl. R⁶ is (1) a 5- to 6-membered heterocyclyl (e.g., heterocycloalkyl, heterocycloalkenyl, or heteroaryl) containing 1-3 hetero ring atoms selected from the group consisting of —O—, —S—, —N═, and —NR^{d}—, where R^{d} is hydrogen, alkyl, cycloalkyl, cycloalkylalkyl, aryl, aralkyl; heterocycloalkyl, heterocycloalkylalkyl, heteroaryl, or heteroaralkyl. This 5- to 6-membered heterocyclyl must be substituted with R^{e} and optionally substituted with one to two R^{f}. R^{e} is oxo, thioxo, alkoxy, alkylsulfinyl, —NH₂, —NH(unsubstituted alkyl), or —N(unsubstituted alkyl)₂, and R^{f} is alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, aminocarbonyl, alkylcarbonylamino, alkylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, heterocycloalkyl, heterocycloalkyloxy, heterocycloalkylsulfanyl, aryl, aryloxy, arylsulfanyl, aroyl, heteroaryl, heteroaryloxy, heteroarylsulfanyl, or heteroaroyl. Alternatively, R⁶ is (2) a fused ring heteroaryl selected from the group consisting of: 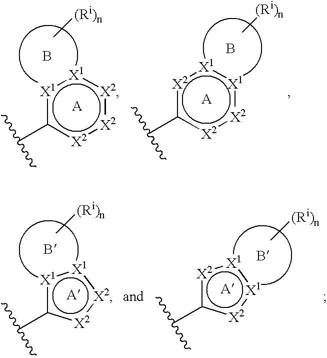
Ring A is an aromatic ring containing 0-4 hetero ring atoms, and ring B is a 5- to 7-membered aromatic or nonaromatic ring containing 0-4 hetero ring atoms; provided that at least one of ring A and ring B contains one or more hetero ring atoms. Ring A′ is an aromatic ring containing 0-4 hetero ring atoms, and ring B′ is a 5- to 7-membered saturated or unsaturated ring containing 0-4 hetero ring atoms; provided that at least one of ring A′ and ring B′ contains one or more hetero ring atoms. Each hetero ring atom of the fused ring heteroaryl is —O—, —S—, —N═, or —NR^{g}—. Specifically, each X¹ ring atom is independently N or C; each X² ring atom is independently —O—, —S—, —N═, —NR^{g}, or —CHR^{h}—. R^{g} is hydrogen, alkyl, cycloalkyl, cycloalkylalkyl, aryl, aralkyl, heterocycloalkyl, heterocycloalkylalkyl, heteroaryl, or heteroaralkyl; and each of R^{h} and Rⁱ is independently alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, or heteroaroyl. n is 0-2; and m is 0-3; provided that when m is greater than or equal to 2, two adjacent R^{a} groups can join together to form a 4- to 8-membered optionally substituted cyclic moiety. That is, the 2-pyridyl ring can fuse with a 4- to 8-membered cyclic moiety to form a moiety such as 7H-[1]pyrindinyl, 6,7-dihydro-5H-[1]pyrindinyl, 5,6,7,8-tetrahydro-quinolinyl, 5,7-dihydro-furo[3,4-b]pyridinyl, or 3,4-dihydro-1H-thiopyrano[4,3-c]pyridinyl. It is further provided that if R⁶ is substituted or unsubstituted naphthyridinyl (e.g., 2-naphthyridinyl), quinolinyl (e.g., 2-quinolinyl or 4-quinolinyl), imidazo[1,2-a]pyridyl, or benzimidazolyl, then —R¹-R²—R³-R⁴ is not H, unsubstituted alkyl, —CH₂—C(O)—N(H)-unsubstituted alkyl, —CH₂—C(O)—N(unsubstituted alkyl)₂, or benzyl.

In one embodiment, R⁶ is a 5- to 6-membered heterocyclyl containing 1-3 hetero ring atoms selected from the group consisting of —O—, —S—, —N—, and —NR^{d}— where R^{d} is hydrogen or alkyl. For example, R⁶ can be a 6-membered heteroaryl containing 1 or 2 hetero ring atoms wherein each hetero ring atom is —N═ or —NR^{d}—. Shown below are two examples of R⁶ as a 6-membered heteroaryl:

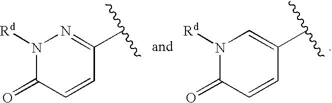

In another embodiment, R⁶ is 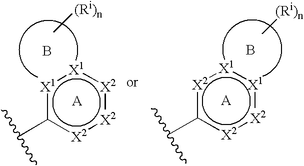
where ring B can be a 5- to 6-membered aromatic or nonaromatic ring. Some examples of such a group are: 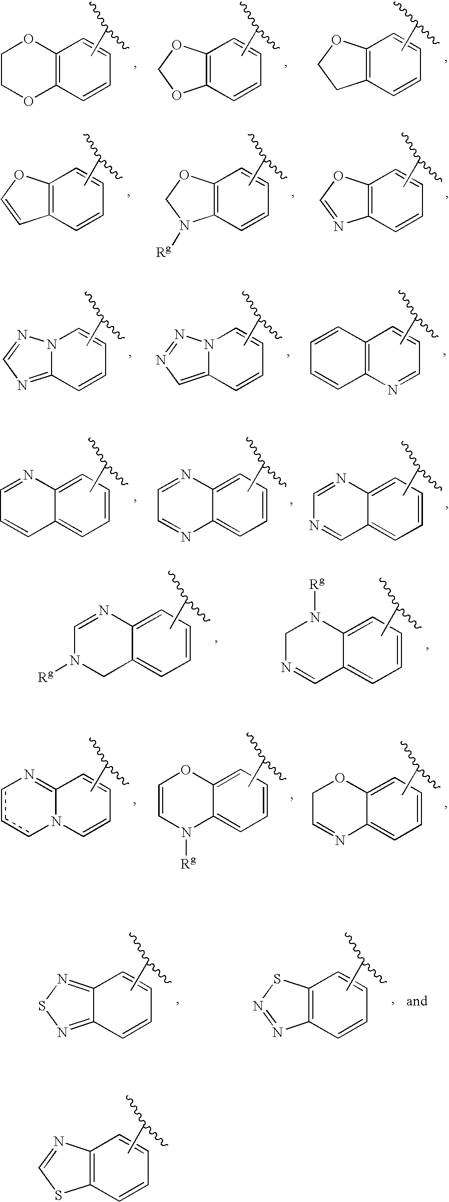
These groups can be unsubstituted or substituted (at one or both rings) with alkyl, alkoxy, halo, oxo, thioxo, amino, alkylsulfinyl, cyano, carboxy, aryl, or heteroaryl and R^{g} is hydrogen, alkyl, cycloalkyl, cycloalkylalkyl, aryl, aralkyl, heterocycloalkyl, heterocycloalkylalkyl, heteroaryl, or heteroaralkyl. Some preferred examples of R⁶ are

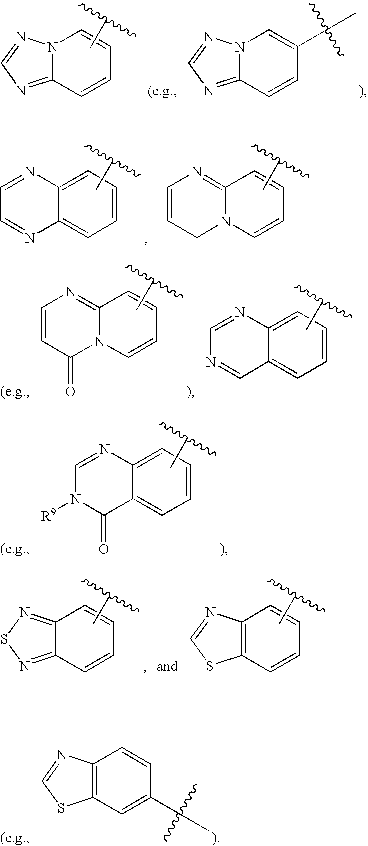

In one embodiment, R⁶ can contain two or three hetero ring atoms (such as oxygen, sulfur, or nitrogen). The para-position of ring A can be occupied by or substituted with one of said hetero ring atoms. Some examples of R⁶ wherein the para-position of its ring A is occupied by a hetero ring atom are: 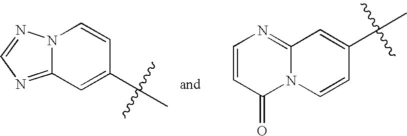
Some examples of R⁶ wherein the para-position of its ring A is substituted with a hetero ring atom are: 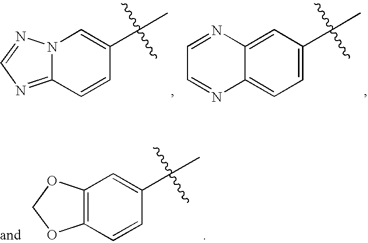
. In one embodiment, the para-position of ring A is substituted with —OR^{j}, —SR^{j}, —O—CO—R^{j}, —O—SO₂—R^{j}, —N(R^{j})₂, —NR^{j}—CO—R^{j}, —NR^{j}—SO₂—R^{j}, or —NR^{j}—C—N(R^{j})₂, where each R^{j} is independently hydrogen, alkyl, cycloalkyl, cycloalkylalkyl, aryl, aralkyl, heterocycloalkyl, heterocycloalkyalkyl, heteroaryl, or heteroaralkyl. Some examples of such R⁶ groups include

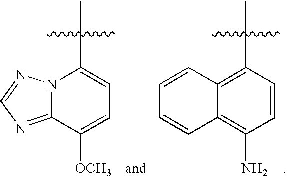

In another embodiment, R⁶ is 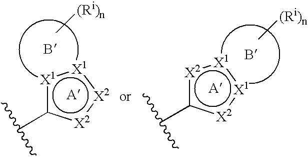
where ring B can be a 5- to 6-membered aromatic or nonaromatic ring. Some examples of
such a group are: 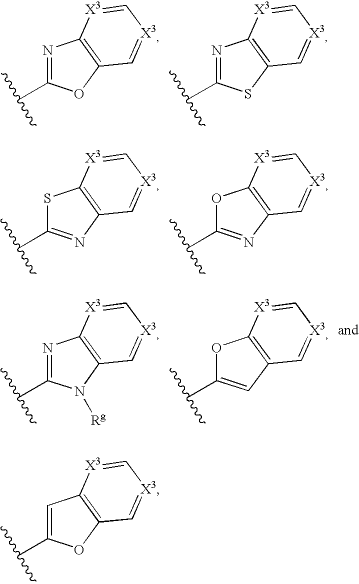
, wherein X³ is independently N or C (i.e., ring B can contain 0-2 nitrogen ring atoms). Note that each R⁶ is optionally substituted with alkyl, alkoxy, halo, oxo, thioxo, amino, alkylsulfinyl, cyano, carboxy, aryl, or heteroaryl. Specific examples of such an R⁶ group are shown below:

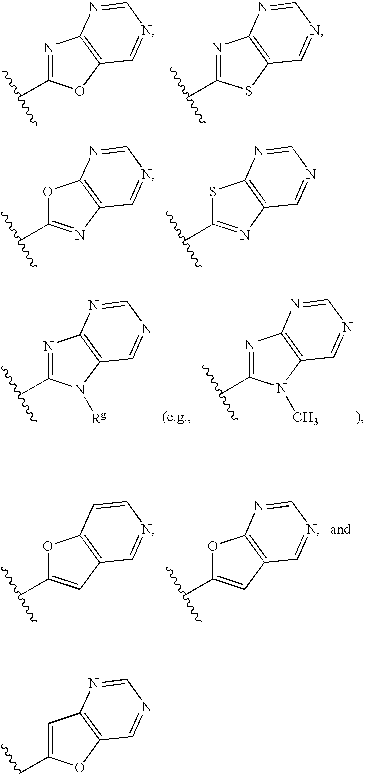

In one embodiment, R¹ is a bond, alkylene, or —(CH₂)₂—O—(CH₂)₂—.

In one embodiment, R² is cycloalkyl, heterocycloalkyl, aryl, heteroaryl, or a bond.

In one embodiment, R³ is —N(R^{b})—C(O)—, —N(R^{b})—S(O)ₚ—, —C(O)—, —C(O)—O—, —O—C(O)—, —C(O)—N(R)—, —S(O)ₚ—, —O—, —S—, —S(O)ₚ—N(R^{b})—, —N(R^{b})—, —N(R^{b})—C(O)—O—, —N(R^{b})—C(O)—N(R)—, or a bond.

In one embodiment, R⁴ is hydrogen, alkyl, heterocycloalkyl, aryl, or heteroaryl.

In one embodiment, R¹ is a bond or alkylene; R² is a bond; R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S(O)ₚ—N(R^{b})—, —N(R^{b})—, or a bond; and R⁴ is hydrogen, alkyl, heterocycloalkyl, aryl, or heteroaryl. In another embodiment, R¹ is —(CH₂)₂—O—(CH₂)₂—; R² piperidinyl, piperazinyl, pyrrolidinyl, tetrahydrofuranyl, tetrahydropyranyl, cyclohexyl, cyclopentyl, bicyclo[2.2.1]heptane, bicyclo[2.2.2]octane, bicyclo[3.2.1]octane, 2-oxa-bicyclo[2.2.2]octane, 2-aza-bicyclo[2.2.2]octane, 3-aza-bicyclo[3.2.1]octane, cubanyl, or 1-aza-bicyclo[2.2.2]octane; R³ is a bond; and R⁴ is hydrogen, alkyl, heterocycloalkyl, aryl, or heteroaryl. In a further embodiment, R¹ is a bond; R² is piperidinyl, piperazinyl, pyrrolidinyl, tetrahydrofuranyl, tetrahydropyranyl, cyclohexyl, cyclopentyl, bicyclo[2.2.1]heptane, bicyclo[2.2.2]octane, bicyclo[3.2.1]octane, 2-oxa-bicyclo[2.2.2]octane, 2-aza-bicyclo[2.2.2]octane, 3-aza-bicyclo[3.2.1]octane, cubanyl, or 1-aza-bicyclo[2.2.2]octane; R³ is —N(R R^{b})—C(O)—, —N(R)—S(O)ₚ—, —C(O)—, —C(O)—O—, —O—C(O)—, —C(O)—N(R R^{b})—, —S(O)ₚ—, —O—, —S—, —S(O)ₚ—N(R^{b}, —N(R R^{b})—, or a bond; and R⁴ is hydrogen, alkyl, heterocycloalkyl, aryl, or heteroaryl. In still a further embodiment, each of R¹, R², and R³ is a bond; and R⁴ is hydrogen or alkyl substituted with cyano.

In one embodiment, R⁵ is hydrogen, unsubstituted alkyl, or halo-substituted alkyl.

In one embodiment, m is 0, 1, or 2. In one embodiment, m is 0 or 1.

In one embodiment, each R^{a} is independently alkyl, alkoxy, alkylsulfinyl, halo, amino, aminocarbonyl, alkoxycarbonyl, cycloalkyl, or heterocycloalkyl. In one embodiment, R^{a} is substituted at the 6-position.

In one embodiment, R⁶ is 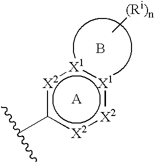
in which ring B is a 5- to 6-membered aromatic or nonaromatic ring; R⁵ is hydrogen, unsubstituted alkyl, or halo-substituted allyl; R⁴ is hydrogen, alkyl, heterocycloalkyl, aryl, or heteroaryl; R³ is —N(R R^{b})—C(O)—, —N(R^{b})—S(O)ₚ—, —C(O)—, —C(O)—O—, —O—C(O)—, —C(O)—N(R R^{b})—, —S(O)ₚ—, —O—, —S—, —S(O)ₚ—N(R^{b})—, —N(R^{b})—, or a bond; R² is a bond; R¹ is a bond or alkylene; and R^{a} is alkyl, alkoxy, alkylsulfinyl, halo, amino, aminocarbonyl, or alkoxycarbonyl; provided that if m is not 0, at least one R^{a} is substituted at the 6-position.

In one embodiment, the para-position of ring A of R⁶ is occupied by or substituted with a hetero ring atom (e.g., O, S, or N) or the para-position of ring A is substituted with —OR^{j}, —SR^{j}, —O—CO—R^{j}, —O—SO₂—R^{j}, —N(R^{j})₂, —NR^{j}—CO—R^{j}, —NR^{j}—SO₂—R^{j}, or —NR^{j}—CO—N(R^{j})₂ where each R^{j} is independently hydrogen, alkyl, cycloalkyl, cycloalkylalkyl, aryl, aralkyl, heterocycloalkyl, heterocycloalkylalkyl, heteroaryl, or heteroaralkyl.

In one embodiment, R⁶ is 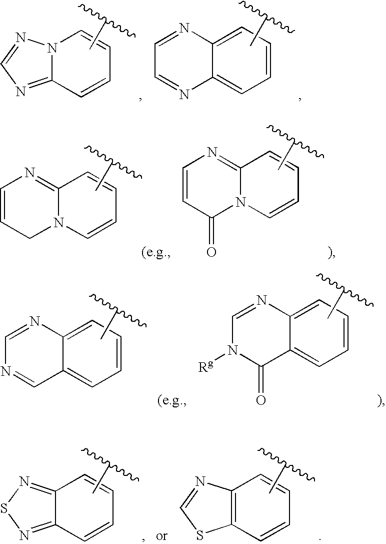
Each of these groups is unsubstituted or substituted (at one or both rings) with alkyl, alkoxy, halo, hydroxy, oxo, amino, alkylsulfinyl, cyano, carboxy, aryl, or heteroaryl. R⁵ is hydrogen, unsubstituted methyl, or trifluoromethyl. R⁴ is hydrogen or alkyl. R³ is —N(R^{b})—C(O)—, —N(R^{b})—S(O)ₚ—, —C(O)—N(R^{b})—, —S(O)ₚ—N(R^{b})—, —N(R^{b})—, or a bond. R² is cycloalkyl or a bond. R¹ is a bond, alkylene, or —(CH₂)₂—O—(CH₂)₂—. In one embodiment, R⁵ is hydrogen and R⁴-R³-R²-R¹— is hydrogen.

It should be noted that the present invention includes compounds having any combination of the groups described herein.

An N-oxide derivative or a pharmaceutically acceptable salt of each of the compounds of formula (I) is also within the scope of this invention. For example, a nitrogen ring atom of the pyrazole core ring or a nitrogen-containing heterocyclyl substituent can form an oxide in the presence of a suitable oxidizing agent such as m-chloroperbenzoic acid or H₂O₂.

A compound of formula (I) that is acidic in nature (e.g., having a carboxyl or phenolic hydroxyl group) can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of formula (I) can be treated with an acid to form acid addition salts. Examples of such an acid include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to a skilled person in the art. The acid addition salts can be prepared by treating a compound of formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of formula (I) can also be, e.g., in a form of achiral compounds, racemic mixtures, optically active compounds, pure diastereomers, or a mixture of diastereomers.

Compounds of formula (I) exhibit surprisingly high affinity to the TGFβ family type I receptors, Alk 5 and/or Alk 4, e.g., with IC₅₀ and Kᵢ value each of less than 10 μM under conditions as described in Example 116 and Example 118, respectively. Some compounds of formula (I) exhibit IC₅₀ and/or Kᵢ value of below 1.0 μM (or even below 0.1 μM).

Compounds of formula (I) can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

In another aspect, the present invention features a pharmaceutical composition comprising a compound of formula (I) (or a combination of two or more compounds of formula (I)) and a pharmaceutically acceptable carrier. Also included in the present invention is a medicament composition including any of the compounds of formula (I), alone or in a combination, together with a suitable excipient.

In a further aspect, the invention features a method of inhibiting the TGFβfamily type I receptors, Alk 5 and/or Alk 4 (e.g., with an IC₅₀ value of less than 10 μM; preferably, less than 1.0 μM; more preferably, less than 0.1 μM) in a cell, including the step of contacting the cell with an effective amount of one or more compounds of formula (I). Also with the scope of the invention is a method of inhibiting the TGFβand/or activin signaling pathway in a cell or in a subject (e.g., a mammal such as human), including the step of contacting the cell with or administering to the subject an effective amount of one or more of a compound of formula (I).

Also within the scope of the present invention is a method of treating a subject or preventing a subject from suffering a condition characterized by or resulted from an elevated level of TGFβ and/or activin activity. The method includes the step of administering to the subject an effective amount of one or more of a compound of formula (I). The conditions include an accumulation of excess extracellular matrix; a fibrotic condition (e.g., scleroderma, lupus nephritis, connective tissue disease, wound healing, surgical scarring, spinal cord injury, CNS scarring, acute lung injury, idiopathic pulmonary fibrosis, chronic obstructive pulmonary disease, adult respiratory distress syndrome, acute lung injury, drug-induced lung injury, glomerulonephritis, diabetic nephropathy, hypertension-induced nephropathy, hepatic or biliary fibrosis, liver cirrhosis, primary biliary cirrhosis, cirrhosis due to fatty liver disease (alcoholic and nonalcoholic steatosis), primary sclerosing cholangitis, restenosis, cardiac fibrosis, opthalmic scarring, fibrosclerosis, fibrotic cancers, fibroids, fibroma, fibroadenomas, fibrosarcomas, transplant arteriopathy, and keloid); TGFβ-induced metastasis of tumor cells; and carcinomas (e.g., squamous cell carcinomas, multiple myeloma, melanoma, glioma, glioblastomas, leukemia, and carcinomas of the lung, breast, ovary, cervix, liver, biliary tract, gastrointestinal tract, pancreas, prostate, and head and neck); and other conditions such as cachexia, hypertension, ankylosing spondylitis, demyelination in multiple sclerosis, cerebral angiopathy and Alzheimer's disease.

As used herein, an “alkyl” group refers to a saturated aliphatic hydrocarbon group containing 1-8 (e.g., 1-6 or 1-4) carbon atoms. An alkyl group can be straight or branched. Examples of an alkyl group include, but are not limited to, methyl, ethyl, propyl, isopropyl, butyl, isobutyl, sec-butyl, tert-butyl, n-pentyl, n-heptyl, and 2-ethylhexyl. An alkyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkylene” is a divalent alkyl group, as defined herein.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkenylene” is a divalent alkenyl group, as defined herein.

As used herein, an “allynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkynylene” is a divalent alkynyl group, as defined herein.

As used herein, an “amino” group refers to —NR^{X}R^{Y} wherein each of R^{X} and R^{Y} is independently hydrogen, alkyl, cycloalkyl, (cycloalkyl)alkyl, aryl, aralkyl, heterocycloalkyl, heterocycloalkyl)alkyl, heteroaryl, or heteroaralkyl. When the term “amino” is not the terminal group (e.g., alkylcarbonylamino), it is represented by —NR^{X}-R^{X} has the same meaning as defined above.

As used herein, an “aryl” group refers to phenyl, naphthyl, or a benzofused group having 2 to 3 rings. For example, a benzofused group includes phenyl fused with one or two C₄₋₈ carbocyclic moieties, e.g., 1, 2, 3, 4-tetrahydronaphthyl, indanyl, or fluorenyl. An aryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl.

As used herein, a “cycloalkyl” group refers to an aliphatic carbocyclic ring of 3-10 (e.g., 4-8) carbon atoms. Examples of cycloalkyl groups include cyclopropyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, and bicyclo[3.2.3]nonyl. A “cycloalkenyl” group, as used herein, refers to a non-aromatic carbocyclic ring of 3-10 (e.g., 4-8) carbon atoms having one or more double bond. Examples of cycloalkenyl groups include cyclopentenyl, 1,4-cyclohexa-di-enyl, cycloheptenyl, cyclooctenyl, hexahydro-indenyl, octahydro-naphthyl, bicyclo[2.2.2]octenyl, and bicyclo[3.3.1]nonenyl. A cycloalkyl or cycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, a “heterocycloalkyl” group refers to a 3- to 10-membered (e.g., 4- to 8-membered) saturated ring structure, in which one or more of the ring atoms is a heteroatom, e.g., N, O, or S. Examples of a heterocycloalkyl group include piperidinyl, piperazinyl, tetrahydropyranyl, tetrahydrofuryl, dioxolanyl, oxazolidinyl, isooxazolidinyl, morpholinyl, octahydro-benzofuryl, octahydro-chromenyl, octahydro-thiochromenyl, octahydro-indolyl, octahydro-pyrindinyl, decahydro-quinolinyl, octahydro-benzo[b]thiophenyl, 2-oxa-bicyclo[2.2.2]octyl, 1-aza-bicyclo[2.2.2]octyl, 3-aza-bicyclo[3.2.1]octyl, anad 2,6-dioxa-tricyclo[3.3.1.0^{3,7}]nonyl. A “heterocycloalkenyl” group, as used herein, refers to a 3- to 10-membered (e.g., 4- to 8-membered) non-aromatic ring structure having one or more double bonds, and wherein one or more of the ring atoms is a heteroatom, e.g., N, O, or S. A heterocycloalkyl or heterocycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloakyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

A “heteroaryl” group, as used herein, refers to a monocyclic, bicyclic, or tricyclic ring structure having 5 to 15 ring atoms wherein one or more of the ring atoms is a heteroatom, e.g., N, O, or S and wherein one ore more rings of the bicyclic or tricyclic ring structure is aromatic. Some examples of heteroaryl are pyridyl, furyl, pyrrolyl, thienyl, thiazolyl, oxazolyl, imidazolyl, indolyl, tetrazolyl, benzofuryl, benzthiazolyl, xanthene, thioxanthene, phenothiazine, dihydroindole, and benzo[1,3]dioxole. A heteroaryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl. A “heteroaralkyl” group, as used herein, refers to an alkyl group (e.g., a C₁₋₄ alkyl group) that is substituted with a heteroaryl group. Both “alkyl” and “heteroaryl” have been defined above.

As used herein, “cyclic moiety” includes cycloalkyl, heterocycloalkyl, cycloalkenyl, heterocycloalkenyl, aryl, or heteroaryl, each of which has been defined previously.

As used herein, a “hetero ring atom” is a non-carbon ring atom of a heterocycloalkyl, heterocycloalkenyl, or heteroaryl and is selected from the group consisting of oxygen, sulfur, and nitrogen.

As used herein, an “acyl” group refers to a formyl group or alkyl-C(═O)— where “alkyl” has been defined previously. Acetyl and pivaloyl are examples of acyl groups.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} is alkyl, cycloalkyl, (cycloalkyl)alkyl, aryl, aralkyl, heterocycloalkyl, (heterocycloalkyl)alkyl, heteroaryl, or heteroaralkyl.

As used herein, a “carboxy” and a “sulfo” group refer to —COOH and —SO₃H, respectively.

As used herein, an “alkoxy” group refers to an alkyl-O— group where “alkyl” has been defined previously.

As used herein, a “sulfoxy” group refers to —O—SO—R^{X} or —SO—R^{X}, where R^{X} has been defined above.

As used herein, a “halogen” or “halo” group refers to fluorine, chlorine, bromine or iodine.

As used herein, a “sulfamoyl” group refers to the structure —SO₂—NR^{X}R^{Y} or —NR^{X}—SO₂—R^{Z} wherein R^{X}, R^{Y}, and R^{Z} have been defined above.

As used herein, a “sulfamide” group refers to the structure —NR^{X}—S(O)₂—NR^{Y}R^{Z} wherein R^{X}, R^{Y}, and R^{Z} have been defined above.

As used herein, a “urea” group refers to the structure —NR^{X}—CO—NR^{Y}R^{Z} and a “thiourea” group refers to the structure —NR^{X}—CS—NR^{Y}R^{Z}. R^{X}, R^{Y}, and R^{Z} have been defined above.

As used herein, an effective amount is defined as the amount which is required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

An antagonist is a molecule that binds to the receptor without activating the receptor. It competes with the endogenous ligand(s) or substrate(s), for binding site(s) on the receptor and, thus inhibits the ability of the receptor to transduce an intracellular signal in response to endogenous ligand binding.

As compounds of formula (I) are antagonists of TGFβ receptor type I (Alk5) and/or activin receptor type I (Alk4), these-compounds are useful in inhibiting the consequences of TGFβ and/or activin signal transduction such as the production of extracellular matrix (e.g., collagen and fibronectin), the differentiation of stromal cells to myofibroblasts, and the stimulation of and migration of inflammatory cells. Thus, compounds of formula (I) inhibit pathological inflammatory and fibrotic responses and possess the therapuetical utility of treating and/or preventing disorders or diseases for which reduction of TGFβ and/or activin activity is desirable (e.g., various types of fibrosis or progressive cancers).

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

Other features and advantages of the invention will be apparent from the following detailed description, and from the claims.

### DETAILED DESCRIPTION OF THE INVENTION

In general, the invention features compounds of formula (I), which exhibit surprisingly high affinity for the TGFβ family type I receptors, Alk 5 and/or Alk 4.

Synthesis of Compounds of Formula (I)

Compounds of formula (I) may be prepared by a number of known methods from commercially available or known starting materials. In one method, a compound of formula (I) are prepared according to Scheme 1 below. Specifically, a pyridine of formula (II), which contains a 2-(α, β-unsaturated carbonyl) substituent can cyclize with hydrazine to form a pyrazole core ring to produce a 2-(pyrazol-3-yl)-pyridine intermediate (III). Note that the pyridine of formula (II) is commercially available (Sigma-Aldrich, St. Louis, Mo., catalog number 51,167-6) or can be prepared by known methods (see, e.g., Jameson, D. and Guise, L. Tetrahedron Letters, 32(18): 1999-2002. The intermediate (III) can be further substituted at the 4-position of the pyrazole core ring with a good leaving group such as iodo by reacting with an iodination reagent (e.g., N-iodosuccinimide) to form a 2-(4-iodo-pyrazol-3-yl)-pyridine (IV). The iodo substituent forms an ideal platform for R⁶ substitutions. For example, the iodo substituent can be converted into a boronic acid substituent (see compound (V) below), which can react with a R⁶-halide (VI) (e.g., an aryl halide or a heteroaryl halide) via Suzuki coupling reaction to form a compound of formula (I). See, e.g., Example 1 below. Other substitution reactions can also be employed to produce a wide range of compounds of formula (I) (see, e.g., via a reaction between the protected iodinated compound (IVa) and phthalic anhydride to form a di-keto intermediate (VI), which can undergo a cyclization reaction with an R^{g}-substituted hydrazine to form a compound (I); for reference, see J. Med. Chem., 44(16): 2511-2522 (2001); see also Examples 3 and 4 below). It should be noted that the pyrazole core ring should be properly protected (see, e.g., the N,N-dimethylaminosulfonyl group of compound (IVa)) to eliminate undesired side reactions.

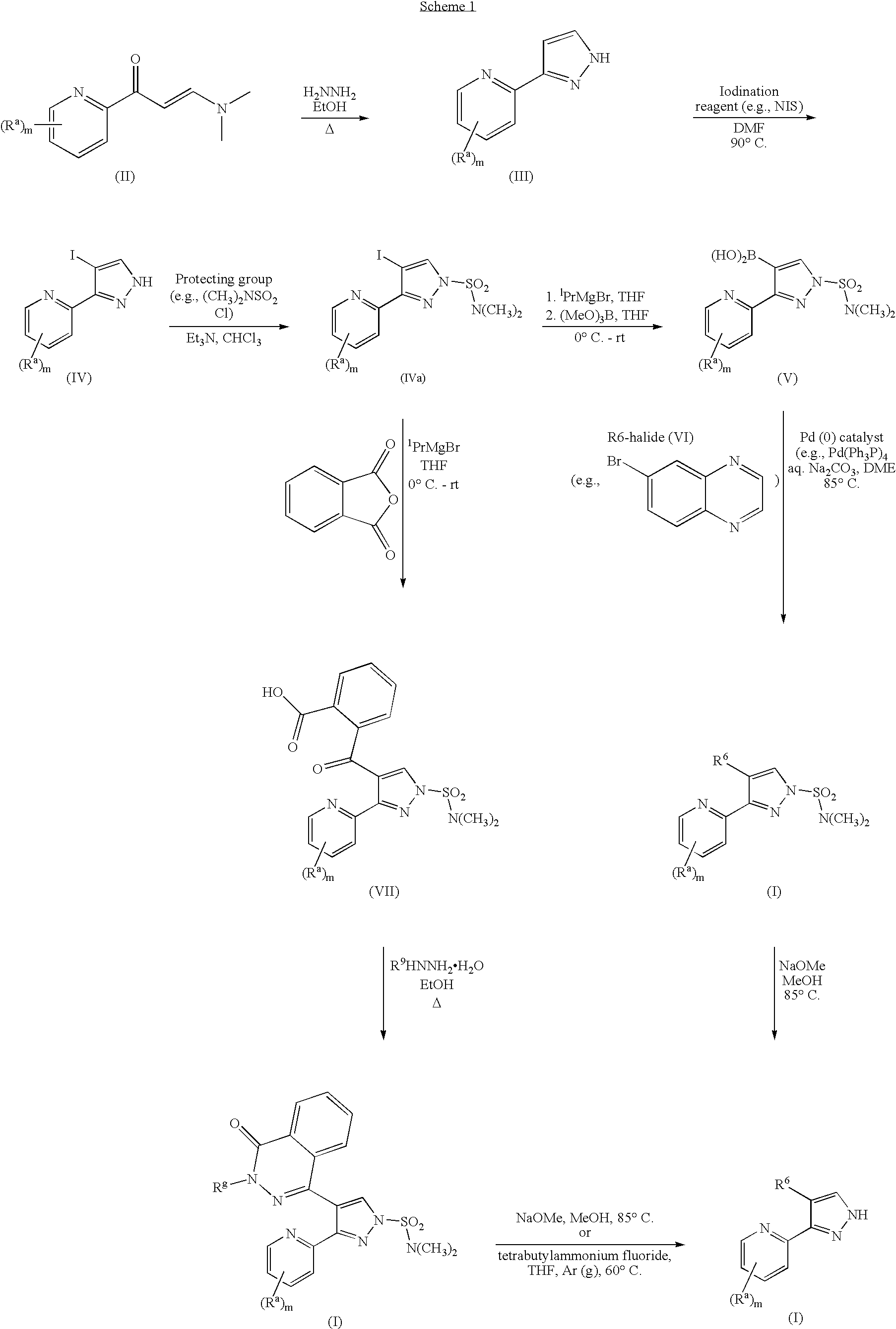

Compounds of formula (VI) are commercially available or can be prepared by known methods. Some exemplary reactions for preparing a compound of formula (VI) are shown below in Scheme 2. See also Examples A-I below.

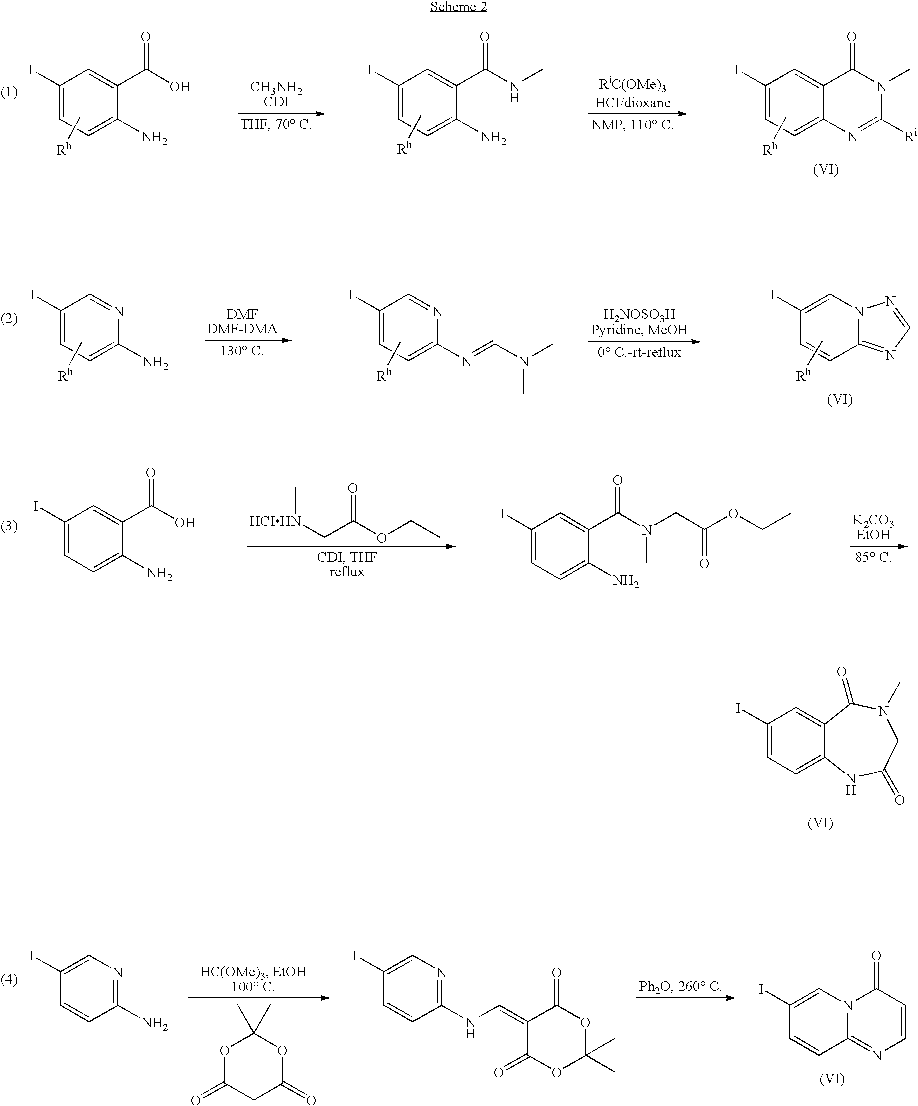

Alternatively, a compound of formula (I) can be formed via a phenylacetyl pyridine compound (IX) as shown in Scheme 3 below. Specifically, a pyridine-carboxyaldehyde compound (VIII) is converted to the N,P acetal intermediate with aniline and diphenylphosphite. This acetal intermediate is then coupled to an aldehyde substituted with R⁶ in basic condition (e.g., Cs₂CO₃) to afford an enamine intermediate, which is hydrolyzed to the ketone intermediate of formula (IX). For reference, see, e.g., Journet et al., Tetrahedron Letters v. 39, p. 1717-1720 (1998). Cyclizing the ketone intermediate (IX) with N,N-dimethylformamide dimethyl acetal (DMFDMA) and hydrazine affords the pyrazole ring of the desired compound of formula (I). See, e.g., Example 5 below. The pyrazole ring of a compound of formula (I) can also be formed by cyclizing the ketone intermediate (IX) with an R⁵-substituted carboxylic acid hydrazide (X). For reference, see, e.g., Chemistry of Heterocyclic compounds 35(11): 1319-1324 (2000).

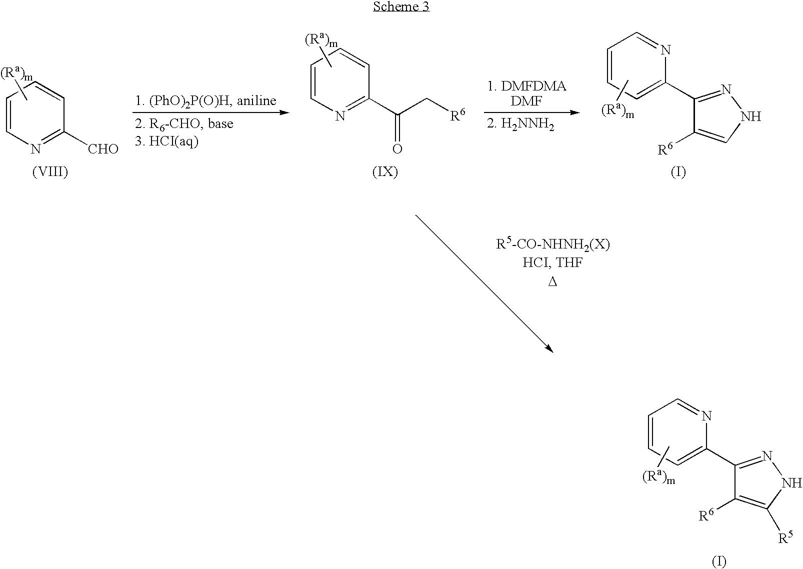

Another method of preparing the intermediate (DC) is depicted in Scheme 4 below. For reference, see, e.g., WO 02/066462, WO 02/062792, and WO 02/062787.

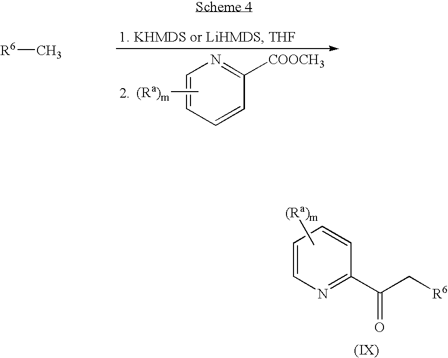

Some methods for preparing a compound of formula (I) wherein —R¹-R²-R³-R⁴ is not hydrogen are shown in Scheme 5 below. In reaction (A) below, a compound of formula (I) wherein the 1-position of the pyrazole core ring is unsubstituted undergoes a substitution reaction with X—R¹-R²-R³-R⁴ where X is a leaving group such as trifluoromethylsulfonate, tosylate, and halide, e.g., Cl, Br, or I (see, e.g., Examples 6-9). Alternatively, a compound of formula (I) wherein the 1-position of the pyrazole core ring is unsubstituted can undergo a conjugate addition reaction as shown in reaction (B) below. As is well known to a skilled person in the art, the electrophile or acceptor in the addition reaction generally contains a double bond connecting to an electron-withdrawing group or a double bond conjugating to groups such as carbonyl, cyano, or nitro. See, e.g., Example 10 below.

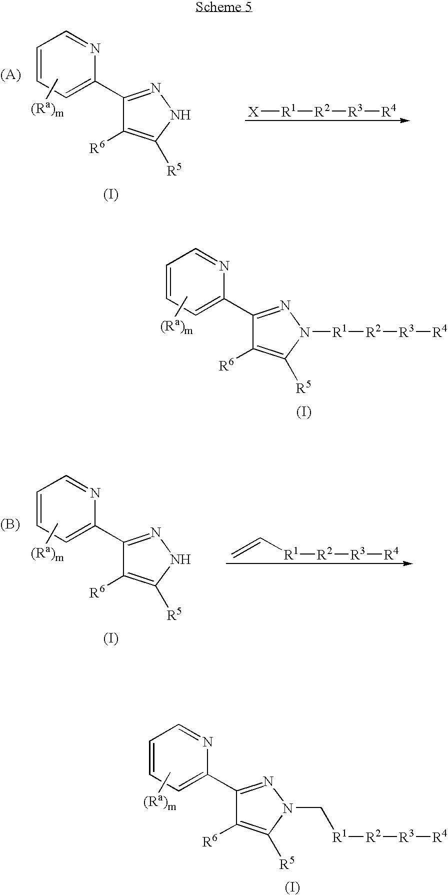

The -R¹-R²-R³-R⁴ group can be further transformed into other functionalities as shown in Scheme 6 below. For example, a compound of formula (I) wherein the -R¹-R²-R³-R⁴ group is cyanoalkyl can be reduced to aminoalkyl, which can be further converted to other functionalities such as heteroaralkyl, heterocycloalkylalkyl, and carboxylic acid. See, e.g., Examples 11-18 below.

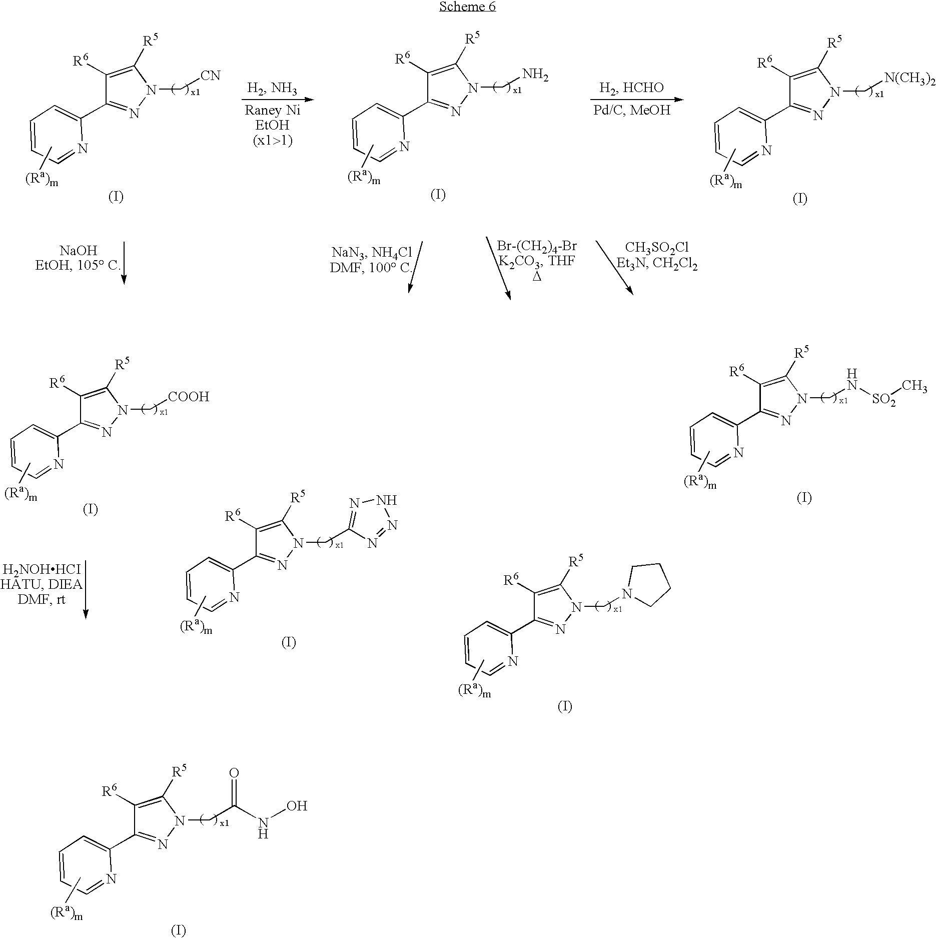

Substituents at the 2-pyridine ring (i.e., R^{a}) can also be converted into other functionalities. For example, a compound of formula (I) wherein R^{a} is bromo (can be obtained by employing a bromo-substituted compound of formula (VIII) (Sigmna-Aldrich, St. Louis, Mo.) can be converted into functionalities such as alkyl, alkenyl, cycloalkyl and the like as described in Examples 19-22.

Likewise, substituents of the R⁶ moiety can be further converted into other functionalities as well. See, e.g., Example 23.

As will be obvious to a skilled person in the art, some starting materials and intermediates may need to be protected before undergoing synthetic steps as described above. For suitable protecting groups, see, e.g., T. W. Greene, Protective Groups in Organic Synthesis, John Wiley & Sons, Inc., New York (1981).

Uses of Compounds of Formula (I)

As discussed above, hyperactivity of the TGFβ family signaling pathways can result in excess deposition of extracellular matrix and increased inflammatory responses, which can then lead to fibrosis in tissues and organs (e.g., lung, kidney, and liver) and ultimately result in organ failure. See, e.g., Border, W. A. and Ruoslahti E. J. Clin. Invest. 90: 1-7 (1992) and Border, W. A. and Noble, N. A. N. Engl. J. Med. 331: 1286-1292 (1994). Studies have been shown that the expression of TGFβ and/or activin mRNA and the level of TGFβ and/or activin are increased in patients suffering from various fibrotic disorders, e.g., fibrotic kidney diseases, alcohol-induced and autoimmune hepatic fibrosis, myelofibrosis, bleomycin-induced pulmonary fibrosis, and idiopathic pulmonary fibrosis. Elevated TGFβ and/or activin is has also been demonstrated in cachexia, demyelination of neurons in multiple sclerosis, Alzheimer's disease, cerebral angiopathy and hypertension.

Compounds of formula (I), which are antagonists of the TGFβ family type I receptors, Alk 5 and/or Alk 4, and inhibit TGFβ and/or activin signaling pathway, are therefore useful for treating and/or preventing disorders or diseases mediated by an increased level of TGFβ and/or activin activity. As used herein, a compound inhibits the TGFβ family signaling pathway when it binds (e.g., with an IC₅₀ value of less than 10 μM; preferably, less than 1 μM; more preferably, less than 0.1 μM) to a receptor of the pathway (e.g., Alk 5 and/or Alk 4), thereby competing with the endogenous ligand(s) or substrate(s) for binding site(s) on the receptor and reducing the ability of the receptor to transduce an intracellular signal in response to the endogenous ligand or substrate binding. The aforementioned disorders or diseases include any conditions (a) marked by the presence of an abnormally high level of TGFβ and/or activin; and/or (b) an excess accumulation of extracellular matrix; and/or (c) an increased number and synthetic activity of myofibroblasts. These disorders or diseases include, but are not limited to, fibrotic conditions such as scleroderma, idiopathic pulmonary fibrosis, glomerulonephritis, diabetic nephropathy, lupus nephritis, hypertension-induced nephropathy, ocular or corneal scarring, hepatic or biliary fibrosis, acute lung injury, pulmonary fibrosis, post-infarction cardiac fibrosis, fibrosclerosis, fibrotic cancers, fibroids, fibroma, fibroadenomas, and fibrosarcomas. Other fibrotic conditions for which preventive treatment with compounds of formula (I) can have therapeutic utility include radiation therapy-induced fibrosis, chemotherapy-induced fibrosis, surgically induced scarring including surgical adhesions, laminectomy, and coronary restenosis.

Increased TGFβ activity is also found to manifest in patients with progressive cancers. Studies have shown that in late stages of various cancers, both the tumor cells and the stromal cells within the tumors generally overexpress TGFβ. This leads to stimulation of angiogenesis and cell motility, suppression of the immune system, and increased interaction of tumor cells with the extracellular matrix. See, e.g., Hojo, M. et al., Nature 397: 530-534 (1999). As a result, the tumors cells become more invasive and metastasize to distant organs. See, e.g., Maehara, Y. et al., J. Clin. Oncol. 17: 607-614 (1999) and Picon, A. et al., Cancer Epidemiol. Biomarkers Prev. 7: 497-504 (1998). Thus, compounds of formula (I), which are antagonists of the TGFβ type I receptor and inhibit TGFβ signaling pathway, are also useful for treating and/or preventing various late stage cancers which overexpress TGFβ. Such late stage cancers include carcinomas of the lung, breast, liver, biliary tract, gastrointestinal tract, head and neck, pancreas, prostate, cervix as well as multiple myeloma, melanoma, glioma, and glioblastomas.

Importantly, it should be pointed out that because of the chronic and in some cases localized nature of disorders or diseases mediated by overexpression of TGFβ and/or activin (e.g., fibrosis or cancers), small molecule treatments (such as treatment disclosed in the present invention) are favored for long-term treatment.

Not only are compounds of formula (I) useful in treating disorders or diseases mediated by high levels of TGFβ and/or activin activity, these compounds can also be used to prevent the same disorders or diseases. It is known that polymorphisms leading to increased TGFβ and/or activin production have been associated with fibrosis and hypertension. Indeed, high serum TGFβ levels are correlated with the development of fibrosis in patients with breast cancer who have received radiation therapy, chronic graft-versus-host-disease, idiopathic interstitial pneumonitis, veno-occlusive disease in transplant recipients, and peritoneal fibrosis in patients undergoing continuous ambulatory peritoneal dialysis. Thus, the levels of TGFβ and/or activin in serum and of TGFβ and/or activin mRNA in tissue can be measured and used as diagnostic or prognostic markers for disorders or diseases mediated by overexpression of TGFβ and/or activin, and polymorphisms in the gene for TGFβ that determine the production of TGFβ and/or activin can also be used in predicting susceptibility to disorders or diseases. See, e.g., Blobe, G. C. et al., N. Engl. J. Med. 342(18): 1350-1358 (2000); Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); and Sugiyama, M. et al., Gastroenterology 114: 550-558 (1998).

Administration of Compounds of Formula (I)

As defined above, an effective amount is the amount which is required to confer a therapeutic effect on the treated patient. For a compound of formula (I), an effective amount can range from about 1 mg/kg to about 150 mg/kg (e.g., from about 1 mg/kg to about 100 mg/kg). Effective doses will also vary, as recognized by those skilled in the art, dependant on route of administration, excipient usage, and the possibility of co-usage with other therapeutic treatments including use of other therapeutic agents and/or radiation therapy.

Compounds of formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in a isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, ferret, lizard, reptile, or bird).

Optionally, compounds of formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid, steroid anti-inflammatory agents, and chemotherapeutics or radiation, as well as agents that antagonize ligand binding or activation of the TGFβ receptors, e.g., anti-TGFβ, anti-TGFβ receptor antibodies, or antagonists of the TGFβ type II receptors.

The invention will be further described in the following examples, which do not limit the scope of the invention described in the claims.

Synthesis of a compound of formula (VI) is described in Examples A-I below. See also Scheme 2 above.

### EXAMPLE A

### 6-Iodo-3-methyl-3H-quinazolin-4-one

To a solution of 5.0 grams (19.0 mmol) of 2-amino-5-iodobenzoic acid in 200 mL dry THF was added 4.6 g (28.5 mmol, 1.5 equiv.) of N,N′-carbonyldiimidazole in one portion with stirring to give a brown mixture. This mixture was heated to reflux for 3 hours and allowed to cool to room temperature. 19 mL (38 mmol, 2 equiv.) of a 2.0 M solution of methylamine in THF was then added to the mixture, which resulted in some gas evolution. The resulting mixture was heated to reflux and stirred for 2 hours, allowed to cool to room temperature and concentrated in vacuo to a purple/brown oil. This oil was dissolved in ethyl acetate, washed three times with 1N NaOH, twice with a 5% citric acid solution, then brine, dried (Na₂SO₄), filtered, and concentrated to a purple solid. This solid was dissolved in hot EtOH, to which water was added until turbid. The reaction mixture was then cooled at 0° C. overnight to give a precipitate. The precipitate was isolated by vacuum filtration, washed with water, and air-dried to give 2-amino-5-iodo-N-methyl-benzamide. Addition of water to the filtrate led to additional precipitate, which was similarly isolated. Total yield of the two crops was 4.55 gram (16.5 mmol, 87%) of 2-amino-5-iodo-N-methyl-benzamide as a pale purple solid. ¹H-NMR (300 MHz, DMSO-d₆, δ): 8.25 (1H, s), 7.71 (1H, s), 7.36 (1H, d, 8.7 Hz), 7.57 (3H, m), 2.69 (3H, d, 6 Hz); m/z=277 [M+H]⁺, 246 [M-NHCH₃]⁺.

To a solution of 2.0 grams (7.2 mmol) of 2-amino-5-iodo-N-methyl-benzamide in 20 mL of NMP was added 6 mL (excess) of trimethyl orthoformate with stirring to give a pale brown solution. To this solution was added 1.0 mL (catalytic) of 4 N HCl in dioxane to give a light-colored precipitate shortly after addition. The mixture was heated to 110° C. for overnight with stirring during which time the reaction mixture became clear. The reaction solution was then cooled and poured into 250 mL ice water to produce an immediate precipitate. The supernatant was neutralized with saturated NaHCO₃ solution (about 5 mL). The solid was isolated by vacuum filtration, washed with water, and air-dried to give 1.40 g (4.9 mmol, 68%) of the product 6-iodo-3-methyl-3H-quinazolin-4-one as a light gray solid. ¹H-NMR (300 MHz, CDCl₃, δ): 8.63 (1H, s), 8.04 (1H, s), 8.00 (1H, d, 8.4 Hz), 7.43 (1H, d, 8.7 Hz), 3.59 (3H, s); m/z: 287 [M+H]⁺.

### EXAMPLE B

### 6-Iodo-[1,2,4]triazolo[1,5-a]pyridine

To a solution of 1.0 gram (4.5 mmol) of 2-amino-5-iodopyridine in 5 mL dry DMF under N2 was added 5 mL (excess) of DMF-dimethylacetal (Sigma-Aldrich, St. Louis, Mo.; 5×1 mL ampules) and the resulting pale yellow solution was heated to 80° C. with stirring for 2 hours. The solution was then allowed to cool and concentrated in vacuo to dryness. The resulting yellow crystalline formamidine, N′-(5-iodo-pyridin-2-yl)-N,N-dimethyl-formamidine, was used in the next step without further purification; ¹H-NMR (300 MHz, CDCl₃, δ): 8.37 (s, 2H), 7.73 (dd, J=2 Hz, 8 Hz, 1H), 6.74 (d, J=9 Hz, 1H), 3.07 (s, 6H); m/z: 276 [M+H]⁺.

To a solution of N′-(5-iodo-pyridin-2-yl)-N,N-dimethyl-formamidine in 8 mL of methanol was added 0.84 mL (10.4 mmol) pyridine and the resulting solution was cooled to 0° C. under nitrogen gas with stirring. To this solution was added 0.66 gram (5.9 mmol) hydroxylamine-O-sulfonic acid to produce a pale yellow suspension. This suspension was allowed to warm to room temperature, then heated to reflux to give a yellow solution. After 16 hours, the solution was allowed to cool to room temperature, during which time crystals began to form. The mixture was cooled to 0° C. (ice bath) for two hours and the crystals were filtered off. After washing extensively with water, the crystals were air-dried to give 0.74 g (3.0 mmol, 67%) of the title compound as very fine, off-white needles; 1H-NMR (300 MHz, CDCl₃, d): 8.88 (1H, s), 8.28 (1H, s), 7.71 (1H, dd, 1.2 Hz, 9.3 Hz), 7.57 (1H, d, 9.3 Hz); m/z: 246 [M+H]⁺.

### EXAMPLE C

### 6-Iodo-2-methyl-[1,2,4]triazolo[1,5-a]pyridine

The title compound was prepared as described in Example B using 5 mL N,N-dimethylacetamide dimethylacetal in 10 mL N,N-dimethylacetamide instead of DMF-dimethyl acetal in DMF. Yield of product was 0.5 g (1.9 mmol, 22%) as very fine, tan-colored crystals. ¹H-NMR (300 MHz, CDCl₃, δ): 8.73 (d, J=1 Hz, 1H), 7.63 (dd, J=1 Hz, 9 Hz, 1H), 7.42 (dd, J=1 Hz, 9 Hz, 1H), 2.58 (s, 3H); m/z: 260 [M+H]⁺.

### EXAMPLE D

### 6-Bromo-5-methyl-[1,2,4]triazolo[1,5-a]pyxidine

Likewise, the title compound was prepared as described above using 1 g (5.3 mmol) 6-amino-3-bromo-2-methylpyridine (Sigma-Aldrich, St. Louis, Mo.) instead of 2-amino-5-iodopyridine. Yield of product was 0.44 g (2.0 mmol, 39%) as fine, white crystals. ¹H-NMR (300 MHz, CDCl₃, δ): 8.34 (s, 1H), 7.65 (d, J=10 Hz, 1H), 7.55 (d, J=10 Hz, 1H), 2.95 (s, 3H); m/z: 213 [M+H]⁺.

### EXAMPLE E

### 7-Iodo-4-methyl-3,4-dihydro-1-H-benzo[e][1,4]diazepine-2,5-dione

To a solution of 1.0 g (3.8 mmol) 2-amino-5-iodobenzoic acid in THF was added 0.925 g (5.7 mmol, 1.5 equiv.) N,N′-carbonyldiimidazole with stirring and the pale yellow solution was heated to reflux for 3 hours, then cooled to ambient temperature. To this solution was added 0.7 mL (4.0 mmol) diisopropylethylamine and 0.875 g (5.7 mmol) sarcosine ethyl ester hydrochloride. The resulting solution was heated to reflux and stirred for overnight. After cooling and concentrating in vacuo, the residue was dissolved in ethyl acetate and washed with 1N NaOH, then 5% citric acid solution, and brine. The organic layer was dried with Na₂SO₄, filtered, and concentrated to a yellow oil. This intermediate, [(2-amino-5-iodo-benzoyl)-methyl-amino]-acetic acid ethyl ester, was used in the next step without further purification. 363 [M+H]⁺, 318 [M-OEt]⁺, 317 [M(cyclized product)+H]⁺.

To a solution of [(2-amino-5-iodo-benzoyl)-methyl-amino]-acetic acid ethyl ester in 50 mL ethanol was added 0.5 g (3.6 mmol) K₂CO₃ and the resulting suspension heated to 85° C. with stirring for 1 hour. The orange mixture was cooled, concentrated in vacuo, and the residue was partitioned between 1N HCl and CH₂Cl₂. The organic layer was separated, dried with Na₂SO₄, filtered, and concentrated to a yellow, foamy solid. This solid was slurried in a small (<5 mL) amount of methanol and the resulting solid filtered, washed with minimal, ice-cold methanol and then water, and finally air-dried to give 0.52 g (1.6 mmol, 43%) of the title compound as an off-white solid. ¹H-NMR (300 MHz, DMSO-d₆, 6): 10.50 (s, 1H), 7.98 (s, 1H), 7.81 (d, J=9 Hz, 1H), 7.57 (d, J=9 Hz, 1H), 3.86 (s, 2H), 3.09 (s, 3H); m/z: 317 [M+H]⁺.

### EXAMPLE F

### 6-Iodo-4-methoxyquinazoline

A suspension of 0.5 g (1.7 mmol) 4-chloro-6-iodoquinazoline (Davos Chemical Corp., Englewood Cliffs, N.J.) in 5 mL of 0.5 M sodium methoxide in methanol was heated to 70° C. in a sealed tube with stirring for 2 hours, then cooled to initiate crystal formation. The mixture was concentrated in vacuo. The residue was suspended in water, filtered, washed with additional water, and air-dried to produce 0.4 g (1.4 mmol, 82%) of the title compound as fine, white needles. ¹H-NMR (300 MHz, CDCl₃, δ): 8.82 (d, J=2 Hz, 1H), 8.55 (d, J=2 Hz, 1H), 8.07 (dt, J=2 Hz, 9 Hz, 1H), 7.67 (dd, J=3 Hz, 9 Hz, 1H), 4.18 (s, 3H); m/z: 287 [M+H]⁺.

### EXAMPLE G

### 6-Iodo-4-aminoquinazoline

A suspension of 0.5 g (1.7 mmol) 4-chloro-6-iodoquinazoline (Davos Chemical Corp., Englewood Cliffs, N.J.) in 10 mL of 7 M ammonia in methanol was heated to 70° C. in a sealed tube with stirring for 90 minutes, then cooled to initiate crystal formation. The mixture was cooled to 0° C., filtered, washed with cold methanol and then petroleum ether, and air-dried to produce 0.39 g (1.4 mmol, 82%) of the title compound as a white solid. ¹H-NMR (300 MHz, DMSO-d₆, δ): 8.64 (d, J=2 Hz, 1H), 8.39 (s, 1H), 8.07 (dd, J=2 Hz, 9 Hz, 1H), 7.85 (br s, 2H), 7.43 (d, J=9 Hz, 1H); m/z: 272 [M+H]⁺.

### EXAMPLE H

### 7-Iodopyrido[1,2-a]pyrimidin-4-one

To a suspension of 2.0 g (9.1 mmol) 2-amino-5-iodopyridine and 1.44 g (10 mmol) of malonic acid cyclic isopropylidene ester in ethanol was added 1.0 mL (9.1 mmol) trimethyl orthoformate and the mixture was heated to 100° C. with stirring. The resulting pale yellow solution began to reflux and the solvent was distilled off to give a bright yellow solid. Heating was continued for 15 minutes until solvent ceased distilling, and the solid was cooled and dissolved in hot acetonitrile to give an orange solution. Upon cooling, dark pink crystals were formed. These crystals were filtered off and recrystallized from acetonitrile to produce 2.2 g (5.9 mmol, 64%) of 5-[(5-iodo-pyridin-2-ylamino)-methylene]-2,2-dimethyl-[1,3]dioxane-4,6-dione as a mixture of pink needles and white filaments. ¹H-NMR (300 MHz, CDCl₃, δ): 11.28 (d, J=13 Hz, 1H), 9.35 (d, J=14 Hz, 1H), 8.62 (d, J=2 Hz, 1H), 8.03 (dd, J=2 Hz, 8 Hz, 1H), 6.86 (d, J=8 Hz, 1H), 1.77 (s, 6H); m/z: 375 [M+H)⁺.

In a 100 mL flask was heated 10 mL phenyl ether to reflux (using a sand bath) with stirring. To the phenyl ether was added 1.0 g (2.7 mmol) of 5-[(5-iodo-pyridin-2-ylamino)-methylene]-2,2-dimethyl-[1,3]dioxane-4,6-dione in one portion to produce an orange solution. This solution was stirred at reflux for 15 minutes (color darkened during the period). The solution was cooled to room temperature and diluted with about 100 mL hexanes to give yellow/brown crystals. These crystals were filtered off and dissolved in hot 95% ethanol, filtered, and cooled to 0° C. The resulting yellow crystals were filtered off and air-dried to give 90 mg of the title compound. The filtrate was concentrated to a yellow solid which was >95% title compound by HPLC. Combined yield of title compound was 320 mg (1.18 mmol, 44%) as a yellow solid. ¹H-NMR (300 MHz, CDCl₃, δ): 9.33 (d, J=2 Hz, 1H), 8.29 (d, J=7 Hz, 1H), 7.89 (dd, J=2 Hz, 9 Hz, 1H), 7.43 (d, J=9 Hz, 1H), 6.49 (d, J=7 Hz, 1H); m/z: 273 [M+H]⁺. For reference, see, e.g., U.S. Pat. No. 3,907,798.

### EXAMPLE I

### 4-Bromo-1-methoxyisoquinoline

A solution of 0.5 g (2.1 mmol) 4-bromo-1-chloroisoquinoline in 10 mL (5 mmol) 0.5 M sodium methoxide in methanol was heated to 70° C. for overnight with stirring, then cooled to a4 mbient temperature and diluted with 30 mL water to produce copious white precipitate. The mixture was cooled to 0° C. for 60 minutes, then filtered, washed with water, and air-dried to produce 0.44 g (1.8 mmol, 88%) of the title compound as a white, waxy solid. ¹H-NMR (300 MHz, CDCl₃, δ): 8.25 (d, J=8 Hz, 1H), 8.18 (s, 1H), 8.06 (d, J=8 Hz, 1H), 7.78 (td, J=1 Hz, 7 Hz, 1H), 7.60 (td, J=1 Hz, 7 Hz, 1H), 4.12 (s, 3H); m/z: 239 [M+H]⁺.

Synthetic procedures illustrated in Schemes 1, 3, 5, and 6 above were employed in the preparation of the title compounds below.

### EXAMPLE 1

### 3-Pyridin-2-yl-4-quinoxalin-6-yl-pyrazole-1-sulfonic acid dimethylamide

Synthesis of the title compound is described in parts (a)-(e) below.

### (a) 2-(1H-Pyrazol-3-yl)-pyridine

To a solution of 10 g (56.7 mmol) of 3-dimethylamino-1-pyridin-2-yl-propenone in 100 “mL absolute ethanol was added 1.96 mL (62.4 mmol, 1.1 equiv.) of anhydrous hydrazine with stirring to give a pale yellow solution. This solution was heated to reflux and stirred overnight, then concentrated to give a tan-colored solid. The solid was then crystallized from ethyl acetate/hexane to give 8.06 g (55.5 mmol, 98%) of 2-(1H-pyrazol-3-yl)-pyridine as tan-colored crystals. ¹H-NMR (300 MHz, CDCl₃, δ): 11.69 (br s, 1H), 8.66 (dd, J=1 Hz, 5 Hz, 1H), 7.76 (d, J=3 Hz, 1H), 7.74 (s, 1H), 7.66 (d, J=2 Hz, 1H), 7.23 (t, J=9 Hz, 1H), 6.81 (d, J=3 Hz, 1H); m/z: 146 [M+H]⁺.

### (b) 2-(4-Iodo-1H-pyrazol-3-yl)pyridine

To an ice-cold, stirred solution of 3.0 g (20.7 mmol) of 2-(1H-pyrazol-3-yl)-pyridine in 25 mL dry DMF was added 4.66 g (20.7 mmol) of N-iodosuccinimide (freshly recrystallized from dioxane/ether) in portions over 10 minutes. The resulting orange solution was warmed to room temperature, then heated to 90° C. overnight with stirring, after which the solution turned dark orange. The solution was partitioned between CH₂Cl₂ and saturated NaHCO₃ solution. The organic solution was washed twice with saturated NaHCO₃, once with water, then brine, and dried with Na₂SO₄. The filtrate was concentrated and the residue was recrystallized twice from ethanol/water to give 3.77 g (13.9 mmol, 67%) of 2-(4-iodo-1H-pyrazol-3-yl)-pyridine as fine, beige-colored crystals. ¹H-NMR (300 MHz, CDCl₃, δ): 11.50 (br s, 1H), 8.64 (dd, J=2 Hz, 6 Hz, 1H), 8.39 (d, J=8 Hz, 1H), 7.82 (td, J=2 Hz, 8 Hz, 1H), 7.69 (s, 1H), 7.30 (qd, J=1 Hz, 5 Hz, 1H); m/z: 272 [M+H]⁺.

### (c) 4-Iodo-3-pyridin-2-yl-pyrazole-1-sulfonic acid dimethylamide

To a solution of 2.46 g (9.1 mmol) of 2-(4-iodo-1H-pyrazol-3-yl)-pyridine in 100 mL CHCl₃ was added 7.0 mL (50 mmol, 5.5 equiv.) of triethylamine with stirring to give a pale yellow solution. This was cooled to 0° C. and 4.9 mL (45.4 mmol, 5 equiv.) of N,N-dimethylsulfamoyl chloride was added slowly over 10 minutes. The yellow solution was allowed to warm to room temperature, then heated to reflux overnight with stirring. The resulting solution was cooled, washed twice with 1N NaOH, then brine, and dried, filtered and concentrated. The residue was dissolved in about 50 mL of 1:1 ethyl acetate/hexanes, passed through a 1.5 inch silica gel plug. The silica plug was washed with an additional 200 mL of 1:1 EA/hex to give a pale orange filtrate. The filtrate was concentrated and the orange residue recrystallized from ethanol/water to give 1.67 g (4.4 mmol, 49%) of 4-iodo-3-pyridin-2-yl-pyrazole-1-sulfonic acid dimethylamide as fine, light orange crystals. ¹H-NMR (300 MHz, CDCl₃, δ): 8.74 (dq, J=0.9 Hz, 1.8 Hz, 4.8 Hz, 1H), 8.11 (s, 1H), 7.95 (dt, J=1.2 Hz, 7.8 Hz, 1H), 7.77 (td, J=1.8 Hz, 7.5 Hz, 1H), 7.33 (qd, J=1.2 Hz, 4.8 Hz, 1H), 3.00 (s, 6H); m/z: 379 [M+H]⁺.

### (d) 1-(N,N-Dimethyl)-sulfamoyl-3-pyridin-2-yl-pyrazole-4-boronic acid

An oven-dried 100 mL flask containing 0.50 g (1.35 mmol) of 4-iodo-3-pyridin-2-yl-pyrazole-1-sulfonic acid dimethylamide was sealed with a septum and flushed with dry nitrogen. The solid was dissolved in 10 mL dry THF with stirring, resulting in a palte orange-colored solution, which was cooled to 0° C. To this solution was slowly added 1.6 mL (1.6 mmol, 1.2 equiv.) of a 1.0 M solution of isopropyl magnesium bromide in THF via syringe to give an orange solution. This solution was allowed to warm to room temperature and stirred for 2 hours, then cannulated into an ice-cold solution of 0.30 mL (2.7 mmol, 2 equiv.) of dry trimethyl borate in 5 mL of dry THF to give a cloudy yellow mixture. This reaction mixture was allowed to warm to room temperature and stirred for 1 hour, then quenched with 5 mL saturated aqueous NH₄Cl solution to give a bilayer. To this was added 15 mL of 1 N NaOH to increase the pH of the aqueous layer to about 10. The layers were separated and the organic solution was extracted once with 1N NaOH. The combined organic solution was acidified to about pH 5-6 with glacial acetic acid, which produced a translucent crystalline precipitate. This mixture containing the crystalline precipitate was cooled to 0° C. for 30 minutes, and the precipitation was filtered, washed with water, and air-dried to give 0.27 g (0.9 mmol, 68%) of 1-(N,N-dimethyl)-sulfamoyl-3-pyridin-2-yl-pyrazole-4-boronic acid as a white solid. ¹H-NM (300 MHz, CDCl₃, δ): 8.74 (br s, 2H), 8.58 (dq, J=0.9 Hz, 1.8 Hz, 4.8 Hz, 1H), 8.42 (s, 1H), 8.37 (dt, J=1.2 Hz, 7.8 Hz, 1H), 7.88 (td, J=1.8 Hz, 8.1 Hz, 1H), 7.38 (qd, J=1.2 Hz, 5.1 Hz, 1H), 3.00 (s, 6H); m/z: 297 [M+H]⁺.

### (e) 3-Pyridin-2-yl-4-quinoxalin-6-yl-pyrazole-1-sulfonic acid dimethylamide

In a pressure tube was combined 425 mg (1.4 mmol) 1-(N,N-dimethyl)-sulfamoyl-3-pyridin-2-yl-pyrazole-4-boronic acid, 200 mg (0.95 mmol) 6-bromoquinoxaline, and 66 mg (0.06 mmol, 6 mol %) of tetrakis-(triphenylphosphine)-palladium (0), which were suspended in 6 mL of ethylene glycol dimethyl ether with stirring. To this reaction mixture was added 3 mL 1M Na₂CO₃ solution before the pressure tube was capped and heated to 85° C. When the reaction mixture reached the desired temperature, it turned into a yellow solution, which was stirred overnight, allowed to cool, and diluted with ethyl acetate. The organic layer was washed 3× with 1N NaOH, then brine, dried (Na₂SO₄), filtered and concentrated to a pale yellow solid. This solid was recrystallized from ethanol to give 260 mg (0.68 mmol, 72%) of 3-pyridin-2-yl-4-quinoxalin-6-yl-pyrazole-1-sulfonic acid dimethylamide as fine, pale orange needles. ¹H-NMR (300 MHz, CDCl₃, δ): 8.83 (s, 2H), 8.50 (dd, 3=0.3 Hz, 4.5 Hz, 1H), 8.26 (s, 1H), 8.13 (d, J=1.8 Hz, 1H), 8.04 (d, J=8.7 Hz, 1H), 7.90 (d, J=7.8 Hz, 1H), 7.77 (td, J=1.8 Hz, 7.5 Hz, 2H), 7.29 (qd, J=0.9 Hz, 4.8 Hz, 1H), 3.09 (s, 6H); m/z: 381 [M+H]⁺.

### EXAMPLE 2

### 6-(3-Pyridin-2-yl-1H-pyrazol-4-yl)-quinoxaline

In a pressure tube was dissolved 100 mg (0.26 mmol) of 3-pyridin-2-yl-4-quinoxalin-6-yl-pyrazole-1-sulfonic acid dimethylamide (see Example 1 above) in 4 mL (excess) of 0.5 M NaOMe in MeOH. The tube was then capped and heated to 85° C. overnight with stirring. The resulting yellow solution was cooled to ambient temperature, neutralized with glacial AcOH, and then purified using reverse-phase preparative HPLC (H₂O/acetonitrile, no buffer; 5% AcCN to 80% AcCN over 10 minutes) to produce 18 mg (0.07 mmol, 25%) of 6-(3-pyridin-2-yl-1H-pyrazol-4-yl)-quinoxaline as a white fluffy solid following lyophilization. ¹H-NMR (300 MHz, CDCl₃, δ): 11.50 (br s, 1H), 8.87 (d, J=1 Hz, 2H), 8.67 (d, J=5 Hz, 1H), 8.21 (d, J=2 Hz, 1H), 8.14 (d, J=9 Hz, 1H), 7.84 (dd, J=2 Hz, 9 Hz, 1H), 7.82 (s 1H), 7.56 (td, J=1 Hz, 7 Hz, 1H), 7.40 (d, J=8 Hz, 1H), 7.25 (m, 1H); m/z: 274 [M+H]⁺.

### EXAMPLE 3

### 4-(4-Oxo-3,4-dihydro-phthalazin-1-yl)-3-pyridin-2-yl-pyrazole-1-sulfonic acid dimethylamide

Synthesis of the title compound is described in parts (a) and (b) below.

### (a) 2-(1-Dimethylsulfamoyl-3-pyridin-2-yl-1H-pyrazole-4-carbonyl)-benzoic acid

A solution of 200 mg (0.53 mmol) of 4-iodo-3-pyridin-2-yl-pyrazole-1-sulfonic acid dimethylamide (see Example 1, subpart (c) above) in 10 mL THF under dry N₂ was cooled to 0° C. with stirring, and 0.9 mL (0.9 mmol) of a 1.0 M solution of isopropyl magnesium bromide in THF was added to produce a yellow solution. This solution was warmed to ambient temperature and stirred for two hours. After the yellow solution was cooled to 0° C., another solution of 130 mg (0.89 mmol) of phthalic anhydride in 5 mL THF was added. The resulting solution was warmed to ambient temperature and stirred for 90 minutes, then diluted with saturated sodium bicarbonate solution (50 mL) and washed once with ethyl acetate. The aqueous layer was acidified to about pH 5 with 1 N HCl and extracted twice with CH₂Cl₂. The organic layers were combined, dried (Na₂SO₄), filtered and concentrated to a yellow-colored oil, which crystallized on standing to give 120 mg (0.30 mmol, 57%) of 2-(1-dimethylsulfamoyl-3-pyridin-2-yl-1H-pyrazole-4-carbonyl)-benzoic acid. This material was used in the next step without further purification. ¹H-NMR (300 MHz, CDCl₃, δ): 8.65 (d, J=5 Hz, 1H), 8.26 (d, J=7 Hz, 1H), 7.96 (m, 2H), 7.80 (td, J=1 Hz, 8 Hz, 1H), 7.71 (m, 2H), 7.50 (m, 1H), 7.40 (s, 1H), 3.02 (s, 6H); m/z: 401 [M+H]⁺.

### (b) 4-(4-Oxo-3,4-dihydro-phthalazin-1-yl)-3-pyridin-2-yl-pyrazole-1-sulfonic acid dimethylamide

To a suspension of 120 mg (0.3 mmol) of 2-(1-dimethylsulfamoyl-3-pyridin-2-yl-1H-pyrazole-4-carbonyl)-benzoic acid in 10 mL of ethanol was added 1 mL (excess) of hydrazine hydrate with stirring. The resulting solution was heated to reflux for 2 hours, cooled, and then concentrated in vacuo to produce a pink/white solid, which was suspended in hot ethanol, and filtered. The filtrate was diluted with water to turbidity. A crystalline precipitate resulted upon cooling at 4° C. overnight. The crystals were filtered off, washed with water, and air-dried to produce 70 mg (0.18 mmol, 59%) of 4-(4-oxo-3,4-dihydro-phthalazin-1-yl)-3-pyridin-2-yl-pyrazole-1-sulfonic acid dimethylamide as fine, pale pink crystals. ¹H-NMR (300 MHz, DMSO-d₆, δ): 12.72 (s, 1H), 8.67 (s, 1H), 8.26 (d, J=7 Hz, 1H), 8.14 (d, J=4 Hz, 1H), 7.99 (d, J=7 Hz, 1H), 7.78 (m, 1H), 7.71 (m, 3H), 7.46 (d, J=7 Hz, 1H), 7.34 (m, 1H), 3.01 (s, 6H); m/z: 397 [M+H]⁺.

### EXAMPLE 4

### 4-(3-Pyridin-2-yl-1H-pyrazol-4-yl)-2H-phthalazin-1-one

Using the same procedure as described in Example 2 above, 4(4-oxo-3,4-dihydro-phthalazin-1-yl)-3-pyridin-2-yl-pyrazole-1-sulfonic acid dimethylamide (see Example 3 above) was treated with excess NaOMe in MeOH to produce the title compound as a white solid. ¹H-NMR (300 MHz, DMSO-d₆, δ): 12.68 (s, 1H), 8.35 (d, J=4 Hz, 1H), 8.28 (d, J=7 Hz, 1H), 8.12 (s, 1H), 7.90 (d, J=8 Hz, 1H), 7.78 (m, 1H), 7.71 (m, 3H), 7.35 (d, J=8 Hz, 1H), 7.24 (t, J=6 Hz, 1H); m/z: 290 [M+H]⁺.

### EXAMPLE 5

### 2-(4-Benzo[1,3]dioxol-5-yl-1H-pyrazol-3-yl)-6-bromo-pyridine

Synthesis of the title compound is described in parts (a) and (b) below.

### (a) 2-Benzo[1,3]dioxol-5-yl-1-(6-bromo-pyridin-2-yl)-ethanone

To a solution of 6-bromo-2-pyridine-carboxylaldehyde (log, 53.76 mmol) in 2-propanol was added aniline (6 mL, 64.51 mmol) and then followed with addition of diphenylphosphite (16.5 mL, 86.02 mmol). The resulting solution was stirred at room temperature for overnight. Precipitations formed in the solution were collected and washed with cold 2-propanol three times and dried to give [(6-bromo-pyridin-2-yl)-phenylamino-methyl]-phosphonic acid diphenyl ester (NP-acetal) as a white solid (19.15 g, 72%). To a solution of the N, P-acetal (37 g, 74.60 mmol) and piperonal (11.2 g, 74.60 mmol) in a mixture of THF (200 mL) and 2-propanol (200 mL) was added cesium carbonate (29 g, 89.52 mmol). The resulting reaction mixture was stirred at room temperature for overnight. A solution of 3M HCl was then added to the reaction mixture and stirred for 3 hours. The solvent of the resulting mixture was evaporated off. The resulting residue was extracted with EtOAc and water. The organic extracts were dried over MgSO₄ and concentrated. The residue was recrystallized in 2-propanol to give the title compound as a white solid (20 g, 84%).

### (b) 2-(4-Benzo[1,3]dioxol-5-yl-1H-pyrazol-3-yl)-6-bromo-pyridine

To a solution of 2-benzo[1,3]dioxol-5-yl-1-(6-bromo-pyridin-2-yl)-ethanone (21.8 g, 68 mmol) in THF (350 mL) was added N,N-dimethylformamide dimethyl acetal (DMFDMA) (23.2 mL, 272 mmol). The mixture was stirred at 60° C. for 3 hours. After the solvent was removed, the resulting residue was dissolved in ethanol (400 mL) and hydrazine (8.9 mL, 409 mmol) was added. The resulting solution was stirred at room temperature for 3 hours and concentrated in vacuo. The residue was purified by silica gel flash column chromatograph to give the title compound (22.5 g, 96%). ¹H-NMR (300 MHz, MeOH-d₄, δ): 7.79-7.20 (m, 4H), 6.92-6.79 (m, 3H), 5.98 (s, 2H).

### EXAMPLE 6

### [4-Benzo[1,3]dioxol-5-yl-3-(6-methyl-pyridin-2-yl)-pyrazol-1-yl]-acetonitrile

To a solution of 150 mg (0.48 mmol) of 2-(4-benzo[1,3]dioxol-5-yl-1H-pyrazol-3-yl)-6-methyl-pyridine prepared in the same manner as described in Example 5 above, using 6-methyl-2-pyridine-carboxylaldehyde instead of 6-bromo-2-pyridine-carboxylaldehyde as starting material in subpart (a)) in THF was added 2.0 mL (1.0 mmol) of 0.5 M NaOMe in MeOH to produce a reaction mixture. The mixture was stirred until 0.07 mL (1.0 mmol) of bromoacetonitrile was added to produce a solution, which was stirred at room temperature overnight. The solution was concentrated to form a residue, which was dissolved in minimal 1:1 MeOH/CH₂Cl₂, loaded onto silica and eluted with 4% MeOH in CH₂Cl₂ to produce 135 mg (0.42 mmol, 88%) of [4-benzo[1,3]dioxol-5-yl-3-(6-methyl-pyridin-2-yl)-pyrazol-1-yl]-acetonitrile as a colorless solid. ¹H-NMR (300 MHz, CDCl₃, δ): 7.61 (s, 1H), 7.50 (q, J=6 Hz, 15 Hz, 1H), 7.11 (m, 2H), 6.77 (s, 1H), 6.75 (s, 2H), 5.95 (s, 2H), 5.18 (s, 2H), 2.60 (s, 3H); m/z: 319 [M+H]⁺.

### EXAMPLE 7

### 4-[4-Benzo[1,3]dioxol-5-yl-3-(6-methyl-pyridin-2-yl)-pyrazol-1-yl]-bicyclo[2.2.2]octane-1-carboxylic acid methyl ester

Synthesis of the title compound is described in parts (a)-(c) below.

### (a) 4-Hydroxy-bicyclo[2.2.2]octane-1-carboxylic acid methyl ester

To a solution of 4-hydroxy-bicyclo[2.2.2]octane-1-carboxylic acid (0.10 g, 0.59 mmol) in methanol (5 mL), was slowly added a solution of (trimethylsilyl)diazomethane in hexane (2.0 M, 1 mL). The reaction mixture was stirred for 2 hours at room temperature. Solvent was then removed to give 4-hydroxy-bicyclo[2.2.2]octane-1-carboxylic acid methyl ester as a yellow solid (0.105 g, 99%). ¹H NMR (300 MHz, CDCl₃, δ): 3.56 (s, 3H), 1.85 (m, 6H), 1.59 (m, 6H).

### (b) 4-Trifluoromethanesulfonyloxy-bicyclo[2.2.2]octane-1-carboxylic acid methyl ester

To a solution of 4-hydroxy-bicyclo[2.2.2]octane-1-carboxylic acid methyl ester (0.105 g, 0.57 mmol) and pyridine (0.10 mL, 1.24 mmol) in dichloromethane (4 mL) was added trifluoromethanesulfonic anhydride (0.10 mL, 0.59 mmol) slowly at 0° C. and stirred for 3 hours. The reaction mixture was diluted with dichloromethane (50 mL). The dichloromethane solution was washed with cold HCl (1 M), followed with 10% NaHCO₃, and then brine. The organic layer was dried over Na₂SO₄ and concentrated to give 4-trifluoromethanesulfonyloxy-bicyclo[2.2.2]octane-1-carboxylic acid methyl ester as a red oil (0.11 g, 61%).

### (c) 4-Trifluoromethanesulfonyloxy-bicyclo[2.2.2]octane-1-carboxylic acid methyl ester

To a solution of 4-trifluoromethanesulfonyloxy-bicyclo[2.2.2]octane-1-carboxylic acid methyl ester (202 mg, 0.64 mmol) and DIEA (223 uL, 1.28 mmol)) in trifluorotoluene (10 mL) was added 2-(4-benzo[1,3]dioxol-5-yl-1H-pyrazol-3-yl)-6-methyl-pyridine (268 mg, 0.96 mmol; see Example 6 above). The mixture was heated to 100° C. for 29 hours and cooled down to room temperature and diluted with CH₂Cl₂. The mixture was then washed with water and brine, dried over MgSO₄, and concentrated. The residue was purified by preparative HPLC to give the title compound, 4-[4-benzo[1,3]dioxol-5-yl-3-(6-methyl-pyridin-2-yl)-pyrazol-1-yl]-bicyclo[2.2.2]octane-1-carboxylic acid methyl ester (10 mg, 4%): ¹H NMR (400 MHz, DMSO-d₆, δ): 8.11 (s, 1H), 7.91 (t, 1H), 7.44 (d, 1H), 7.41 (d, 1H), 7.02 (s, 1H), 6.87-6.82 (m, 2H), 6.00 (s, 2H), 3.62 (s, 3H), 2.54 (s, 3H), 2.16-2.13 (m, 6H), 1.98-1.94 (m, 6H); MS (ESP+) m/z 446.3 (M+1) and an isomer of the title compound.

### EXAMPLE 8

### 4-(2-{2-[4-Benzo[1,3]dioxol-5-yl-3-(6-methyl-pyridin-2-yl)-pyrazol-1-yl]-ethoxy}-ethoxy)-bicyclo[2.2.2]octane-1-carboxylic acid methyl ester

2-(4-Benzo[1,3]dioxol-5-yl-1H-pyrazol-3-yl)-6-methyl-pyridine (0.146 g, 0.52 mmol; see Example 6 above) was added to a solution of 4-trifluoromethanesulfonyloxy-bicyclo[2.2.2]octane-1-carboxylic acid methyl ester (0.11 g, 0.35 mmol; see Example 7, subparts (a) and (b) above) and diisopropylethylamine (0.09 g, 0.70 mmol) in 1,4-dioxane (5 mL). The reaction mixture was heated to 100° C. with stirring for 30 hours. Solvent was then removed. The residue was partitioned between ethyl acetate and water. The organic layer was washed with brine and dried over Na₂SO₄. The residue obtained from concentration was purified by preparative HPLC to produce the title compound, 4-(2-{2-[4-benzo[1,3]dioxol-5-yl-3-(6-methyl-pyridin-2-yl)-pyrazol-1-yl]-ethoxy}-ethoxy)-bicyclo[2.2.2]octane-1-carboxylic acid methyl ester (0.05 g, 27%): ¹H NMR (300 MHz, Methanol-d₄, δ): 8.26 (t, 1H, J=8.1 Hz), 8.01 (s, 1H), 7.74 (d, 1H, J=7.8 Hz), 7.61 (d, 1H, J3=8.1 Hz), 6.84 (m, 3H), 6.00 (s, 2H), 4.48 (m, 2H), 3.92 (m, 2H), 3.57 (m, 2H), 3.31 (m, 2H), 2.84 (s, 3H), 1.79 (m, 6H), 1.58 (m, 6H). MS (ES+) m/z 534.2 (M+1) and an isomer of the title compound.

### EXAMPLE 9

### 4-(2-{2-[4-Benzo[1,3]dioxol-5-yl-3-(6-methyl-pyridin-2-yl)-pyrazol-1-yl]-ethoxy}-ethoxy)-bicyclo[2.2.2]octane-1-carboxylic acid

A solution of 4-(2-{2-[4-benzo[1,3]dioxol-5-yl-5-(6-methyl-pyridin-2-yl)-pyrazol-1-yl]-ethoxy}-ethoxy)-bicyclo[2.2.2]octane-1-carboxylic acid methyl ester (0.02 g, 0.037 mmol; see Example 8 above) in concentrated hydrochloric acid (3 mL) was stirred at room temperature for 18 hours. The reaction mixture was then quenched with concentrated ammonium hydroxide. Water was removed under vacuum to give a white solid, which was washed with methylene chloride, and the methylene chloride wash was concentrated. Preparative HPLC purification gave the title compound as a yellow solid (0.002 g, 11%). ¹H NMR (300 MHz, Methanol-d₄, δ): 8.28 (t, 1H, J=8.1 Hz), 8.03 (s, 1H), 7.75 (d, 1H, J=7.8 Hz), 7.62 (d, 1H, J=7.8 Hz), 6.85 (m, 3H), 6.00 (s, 2H), 4.49 (m, 2H), 3.93 (m, 2H), 3.58 (m, 2H), 3.48 (m, 2H), 2.84 (s, 3H), 1.79 (m, 6H), 1.59 (m, 6H); MS (ESP+) m/z 520.4 (M+1).

### EXAMPLE 10

### 3-[4-Benzo[1,3]dioxol-5-yl-3-(6-methyl-pyridin-2-yl)-pyrazol-1-yl]-propionitrile

To a solution of 250 mg (0.9 mmol) 2-(4-benzo[1,3]dioxol-5-yl-1H-pyrazol-3-yl)-6-methyl-pyridine (see Example 6 above) in EtOH was added 0.25 mL of a 50% aq. KOH solution with stirring to give a pink precipitate. To this precipitate was added 0.12 mL (1.8 mmol) of acrylonitrile. The resulting solution was stirred overnight, and then filtered. The filtrate was concentrated to form a residue, which was dissolved in minimal 1:1 MeOH/CH₂Cl₂, loaded onto silica and eluted with 4% MeOH in CH₂Cl₂ to produce 160 mg (0.48 mmol, 54%) of 3-[4-benzo[1,3]dioxol-S-yl-3-(6-methyl-pyridin-2-yl)-pyrazol-1-yl]-propionitrile as a colorless solid. ¹H-NMR (400 MHz, DMSO-d₆, δ): 8.04 (s, 1H), 7.61 (t, J=12 Hz, 1H), 7.39 (d, J=6 Hz, 1H), 7.20 (d, J=6 Hz, 1H), 7.05 (s, 1H), 6.83 (s, 2H), 5.97 (s, 2H), 4.44 (t, J=6 Hz, 2H), 3.13 (t, J=6 Hz, 2H), 2.41 (s, 3H); m/z: 333 [M+H]⁺.

### EXAMPLE 11

### 3-[4-Benzo[1,3]dioxol-5-yl-3-(6-methyl-pyridin-2-yl)-pyrazol-1-yl]-propylamine

To a solution of 130 mg (0.39 mmol) of 3-[4-benzo[1,3]dioxol-5-yl-3-(6-methyl-pyridin-2-yl)-pyrazol-1-yl]-propionitrile (see example 10, above) in 4 mL of EtOH was added 2 mL (excess) of a 2 M solution of ammonia in EtOH with stirring. To this resulting solution was added a catalytic amount of Raney nickel that was prewashed with EtOH. The mixture was subjected to 40 psi of hydrogen gas with vigorous stirring for 2 hours, after which it was filtered through a plug of Celite. The filtrate was concentrated to produce 135 mg (quantitative) of the title compound as a colorless oil which was used in the following transformations without further purification; m/z 337 [M+H]⁺.

### EXAMPLE 12

### 3-(3-Pyridin-2-yl-4-quinolin-4-yl-pyrazol-1-yl)-propylamine

To a solution of 130 mg (0.39 mmol) of 3-(3-pyridin-2-yl-4-quinolin-4-yl-pyrazol-1-yl)-propionitrile (prepared by reacting 4-(3-pyridin-2-yl-1H-pyrazol-4-yl)-quinoline with acrylnitrile) in 4 mL of EtOH was added 2 mL (excess) of a 2 M solution of ammonia in EtOH with stirring. To this resulting solution was added a catalytic amount of Raney nickel that was prewashed with EtOH. The mixture was subjected to 40 psi of hydrogen gas with vigorous stirring for two hours, after which it was filtered through a plug of Celite. The filtrate was concentrated to give 135 mg (quantitative) of the title compound as a colorless oil, which was used in the following transformations without further purification. A 30 mg portion was dissolved in 5 mL CH₂Cl₂, 1.0 mL of 1M HCl in ether added to give a precipitate, the precipitate isolated by filtration and air-dried to give the title compound as its hydrochloride salt. ¹H-NMR (300 MHz, DMSO-d₆, δ): 9.18 (d, J-=4 Hz, 1H), 8.46 (s, 1H), 8.39 (d, J=8 Hz, 1H), 8.17 (br s, 2H), 8.10 (d, J-=5 Hz, 1H), 8.06 (t, J-=7 Hz, 1H), 8.01 (d, J-=8 Hz, 1H), 7.87 (m, 3H), 7.72 (t, J=7 Hz, 1H), 7.26 (t, J=6 Hz, 1H), 4.47 (t, J=7 Hz, 2H), 2.91 (m, 2H), 2.27 (m, 2H); m/z 330.8 [M+H]⁺.

### EXAMPLE 13

### N-{3-[4-Benzo[1,3]dioxol-5-yl-3-(6-methyl-pyridin-2-yl)-pyrazol-1-yl]-propyl}-methanesulfonamide

To a solution of 135 mg (0.39 mmol) of 3-[4-benzo[1,3]dioxol-5-yl-3-(6-methyl-pyridin-2-yl)-pyrazol-1-yl]-propylamine (see Example 11 above) in CH₂Cl₂ was added 0.14 mL (1.0 mmol) of triethylamine with stirring, followed by 0.06 mL (0.8 mmol) of methanesulfonyl chloride to give a yellow solution. This yellow solution was stirred at room temperature for 2 hours, then concentrated, redissolved in MeOH and purified by preparative HPLC (H₂O/acetonitrile, no buffer; 5% AcCN to 80% AcCN over 10 minutes) to produce 21 mg of the title compound as a pale yellow solid. ¹H-NMR (300 MHz, CDCl₃, δ): 7.97 (d, J=4 Hz, 1H), 7.55 (s, 1H), 7.40 (m, 2H), 7.13 (s, 1H), 6.79 (d, J=8 Hz, 2H), 6.00 (s, 2H), 4.46 (t, J=6 Hz, 2H), 3.20 (m, 5H), 2.96 (s, 3H), 2.36 (t, J=6 Hz, 2H); m/z: 415 [M+H]⁺.

### EXAMPLE 14

### Dimethyl-[3-(3-pyridin-2-yl-4-quinolin-4-yl-pyrazol-1-yl)-propyl]-amine

To a solution of 50 mg (0.15 mmol) of 3-(3-pyridin-2-yl-4-quinolin-4-yl-pyrazol-1-yl)-propylamine (free base, see Example 12 above) in 3 mL methanol was added 0.025 mL of a 37% aqueous solution of formaldehyde with stirring followed by a catalytic amount of 10% palladium on carbon to give a black mixture. This mixture was placed under 50 psi of hydrogen gas and stirred at room temperature overnight, then purged and filtered through a plug of Celite. The filtrate was concentrated and purified by preparative HPLC (H₂O/acetonitrile, no buffer; 5% AcCN to 80% AcCN over 10 minutes) to produce 17 mg (0.048 mmol, 32%) of the title compound as a colorless solid. ¹H-NMR (300 MHz, CDCl₃, δ): 8.49 (d, J=4 Hz, 1H), 8.07 (d, J=4 Hz, 1H), 7.74 (d, J=8 Hz, 1H), 7.43 (d, J=7 Hz, 1H), 7.29 (d, J=8 Hz, 1H), 6.95 (m, 5H), 6.68 (dd, J=2 Hz, 5 Hz, 1H), 3.98 (t, J=7 Hz, 2H), 1.99 (t, J=7 Hz, 2H), 1.87(s, 6H), 1.81 (t, J=7 Hz, 2H); m/z: 319 [M+H]⁺.

### EXAMPLE 15

### 4-[3-Pyridin-2-yl-1-(3-pyrrolidin-1-yl-propyl)-1H-pyrazol-4-yl]-quinoline, HCl salt

To a solution of 50 mg (0.15 mmol) of 3-(3-pyridin-2-yl-4-quinolin-4-yl-pyrazol-1-yl)-propylamine (free base, see Example 12 above) in 5 mL THF was added 138 mg (1 mmol) K₂CO₃ followed by 0.04 mL (0.32 mmol) of 1,4-dibromobutane to give a colorless mixture. After being heated at reflux overnight, the resulting reaction mixture was filtered, concentrated and purified by preparative HPLC (H₂O/acetonitrile, no buffer; 5% AcCN to 80% AcCN over 10 minutes) to produce a colorless solid, which was converted to its HCl salt by dissolving it in 5 mL CH₂Cl₂ and adding 1.2 equivalents of 1M HCl in Et₂O. The resulting solution was then concentrated to 11 mg of the title compound as a pale yellow solid. ¹H-NMR (300 MHz, DMSO-d₆, δ): 11.02 (br s, 1H), 9.17 (d, J=5 Hz, 1H), 8.45 (s, 1H), 8.36 (d, J=9 Hz, 1H), 8.06 (m, 3H), 7.89 (m, 3H), 7.71 (t, J-=7 Hz, 1H), 7.25 (t, J=5 Hz, 1H), 4.47 (t, J=6 Hz, 2H), 3.55 (d, J=5 Hz, 2H), 3.25 (q, J=2 Hz, 6 Hz, 2H), 3.00 (m, 2H), 2.39 (t, J=7 Hz, 2H), 1.95 (m, 4H); m/z: 385 [M+H]⁺.

### EXAMPLE 16

### 4-{3-Pyridin-2-yl-1-[2-(2H-tetrazol-5-yl)-ethyl]-1H-pyrazol-4-yl}-quinoline

To a mixture of 70 mg (0.20 mmol) 3-(3-pyridin-2-yl-4-quinolin-4-yl-pyrazol-1-yl)-propionitrile (see Example 12 above), 30 mg (0.44 mmol) sodium azide, and 24 mg (0.44 mmol) ammonium chloride in a high-pressure tube was added 3 mL dry DMF. The resulting suspension was heated to 100° C. and stirred overnight, then cooled and concentrated. The residue was dissolved in 5 mL of a 1 M aqueous Na₂CO₃ solution, washed twice with CH₂Cl₂, then the volume was reduced by half in vacuo and neutralized with glacial AcOH. The resulting mixture was purified by preparative HPLC (H₂O/acetonitrile, no buffer; 5% AcCN to 80% AcCN over 10 minutes) to produce 26 mg (0.07 mmol, 35%) of the title compound as a fluffy, white solid. ¹H-NMR (300 MHz, DMSO-d₆, δ): 8.79 (d, J=5 Hz, 1H), 8.09 (d, J=4 Hz, 1H), 8.03 (s, 1H), 7.99 (d, J=8 Hz, 1H), 7.82 (d, J=8 Hz, 1H), 7.75 (td, J=2 Hz, 8 Hz, 1H), 7.69 (m, 1H), 7.39 (td, J=1 Hz, 8 Hz, 1H), 7.28 (d, J=4 Hz, 1H), 7.13 (t, J=5 Hz, 1H), 4.56 (t, J=7 Hz, 2H), 3.31 (t, J=7 Hz, 2H); m/z: 369 [M+H]⁺.

### EXAMPLE 17

### 3-(3-Pyridin-2-yl-4-quinolin-4-yl-pyrazol-1-yl)-propionic acid

To a solution of 110 mg (0.34 mmol) 3-(3-pyridin-2-yl-4-quinolin-4-yl-pyrazol-1-yl)-propionitrile (see Example 12 above) in 5 mL ethanol was added 10 mL of a 5 M aqueous NaOH solution to give a cloudy mixture. This reaction mixture was heated to 105° C. with stirring overnight to give a colorless solution. The resulting reaction mixture was cooled to room temperature and neutralized with glacial acetic acid to give a white precipitate. The precipitate was separated and the filtrate was extracted twice with 50 mL CH₂Cl₂. The organics were combined and dried (Na₂SO₄), filtered, and concentrated to a yellow solid. This solid was combined with the precipitate and purified by preparative HPLC (H₂O/acetonitrile, no buffer; 5% AcCN to 80% AcCN over 10 minutes) to produce 25 mg (0.07 mmol, 21%) of the title compound as a white solid. ¹H-NMR (300 MHz, DMSO-d₆, δ): 12.48 (br s, 1H), 8.80 (d, J=4 Hz, 1H), 8.09 (s, 1H), 8.07 (t, J=5 Hz, 1H), 8.01 (d, J=8 Hz, 1H), 7.71 (m, 4H), 7.38 (td, J=2 Hz, 8 Hz, 1H), 7.29 (d, J=4 Hz, 1H), 7.14 (td, J=1 Hz, 6 Hz, 1H), 4.49 (t, J=7 Hz, 2H), 2.96 (t, J=7 Hz, 2H); m/z: 345 [M+H]⁺.

### EXAMPLE 18

### N-Hydroxy-3-(3-pyridin-2-yl-4-quinolin-4-yl-pyrazol-1-yl)-propionamide

To a solution of 35 mg (0.1 mmol) 3-(3-pyridin-2-yl-4-quinolin-4-yl-pyrazol-1-yl)-propionic acid (see Example 17 above) in 2 mL DMF was added 14 mg (0.2 mmol) hydroxylamine hydrochloride, 46 mg (0.12 mmol) of O-(7-azabenzotriazol-1-yl)-1,1,3,3-tetramethyluronium hexafluorophosphate (HATU), then 0.09 mL (0.5 mmol) diisopropylethylamine to give a yellow solution. This was stirred at room temperature for 2 hours and then purified by preparative HPLC (H₂O/acetonitrile, no buffer; 5% AcCN to 80% AcCN over 10 minutes) to produce 2 mg (0.06 mmol, 6%) of the title compound as a white solid. ¹H-NMR (300 MHz, CDCl₃, δ): 11.17 (br s, 1H), 9.65 (br s, 1H), 8.68 (d, J=4 Hz, 1H), 8.40 (d, J=4 Hz, 1H), 8.07 (d, J=8 Hz, 1H), 7.63 (m, 4H), 7.38 (d, J=7 Hz, 1H), 7.08 (m, 3H), 4.57 (t, J=7 Hz, 2H), 2.92 (t, J=7 Hz, 2H); m/z: 360 [M+H]⁺.

### EXAMPLE 19

### 2-(4-Benzo[1,3]dioxol-5-yl-1H-pyrazol-3-yl)-6-vinyl-pyridine

Synthesis of the title compound is described in parts (a) and (b) below.

### (a) 4-Benzo[1,3]dioxol-5-yl-3-(6-bromo-pyridin-2-yl)-pyrazole-1-sulfonic acid dimethylamide

To a solution of 2-(4-benzo[1,3]dioxol-5-yl-1H-pyrazol-3-yl)-6-bromo-pyridine (11.8 g, 34 mmol; see Example 5 above) in CH₂Cl₂ (250 mL) was added dimethylsulfamoyl chloride (14.7 mL, 136 mmol), triethylamine (28.8 mL, 204 mmol) and DMAP (1.0 g). The mixture was stirred at 60° C. for 3 days, the solvent was then evaporated off. Ethyl acetate (150 mL) was added to the residue, and the insoluble solid was filtered off. The filtrate was concentrated and purified by silica gel flash column chromatograph to give the title compound as a yellow solid (12.1 g, 78%).

### (b) 2-(4-Benzo[1,3]dioxol-5-yl-1H-pyrazol-3-yl)-6-vinyl-pyridine

A mixture 4-benzo[1,3]dioxol-5-yl-3-(6-bromo-pyridin-2-yl)-pyrazole-1-sulfonic acid dimethylamide (210 mg, 0.47 mmol), tributyl(vinyl)tin (295 mg, 0.93 mmol), tetrakis-(triphenylphosphino)palladium (27 mg, 0.024 mmol) in THF (2 mL) was heated in a sealed tube at 120° C. for overnight. The reaction was cooled to room temperature and extracted with CH₂Cl₂ and saturated sodium carbonate. The orgainc layer was dried over MgSO₄ and concentrated. The resulting residue was purified on silica gel column with 0-5% EtOAc/CH₂Cl₂ to give 4-benzo[1,3]dioxol-5-yl-3-(6-vinyl-pyridin-2-yl)-pyrazole-1-sulfonic acid dimethylamide (183 mg, 99%). 100 mg of this sulfonic acid dimethylamide (0.25 mmol) was then dissolved in a mixture of THF (2 mL) and EtOH (8 mL) and a solution of NaOEt in EtOH (23%, 1 mL) was added. The resulting solution was heated to reflux for overnight. The reaction was cooled to room temperature and concentrated. The resulting residue was filtered through a short silica gel column and washed with THF. The filtrates were concentrated and redissolved in DMSO. The resulting solution was purified by semi-preparative HPLC to produce the title compound (30 mg, 41%). MS (ESP⁺) m/z 292.3 (M+1). ¹H NMR (300 MHz, MeOH-d₄, δ): 8.30 (t, 1H), 815 (d, 1H), 7.96 (s, 1H), 7.68 (d, 1H), 7.16 (dd, 1H), 6.90-6.82 (m, 3H), 6.57 (d, 1H), 6.05 (d, 1H), 6.02 (s, 2H).

### EXAMPLE 20

### 2-(4-Benzo[1,3]dioxol-5-yl-1H-pyrazol-3-yl)-6-ethyl-pyridine

A suspension of 2-(4-benzo[1,3]dioxol-5-yl-1H-pyrazol-3-yl)-6-vinyl-pyridine (20 mg, 0.069 mmol; see Example 19 above) and Pd/C (10%, 50 mg) in a mixture of MeOH (5 mL) and EtOAc (5 mL) was stirred at room temperature under 1 atmosphere of hydrogen gas for 1 hour. The residue was filtered off through a Celite cake and washed with THF. The filtrate was concentrated and purified by semi-preparative HPLC to produce the title compound (10 mg, 50%). MS (ESP⁺) m/z 294.1 (+1). ¹H NMR (300 MHz, MeOH-d₄, δ): 8.28 (t, 1H), 7.95 (s, 1H), 7.76 (d, 1H), 7.65 (d, 1H), 6.90-6.82 (m, 3H), 6.01 (s, 2H), 3.11 (q, 2H), 1.43 (t, 3H).

### EXAMPLE 21

### 2-(4-Benzo[1,3]dioxol-5-yl-1H-pyrazol-3-yl)-6-cyclopropyl-pyridine

A solution of cyclopropylmagnesium bromide in THF (0.5 M, 0.5 mL) was added dropwise to the solution of ZnCl₂ in THF (0.5 M, 0.5 mL) at −78° C. with stirring. The resulting suspension was allowed to warm up to room temperature and stirring was continued for an additional 1.5 hours. The suspension was then transferred to a sealed tube together with 4-benzo[1,3]dioxol-5-yl-3-(6-bromo-pyridin-2-yl)-pyrazole-1-sulfonic acid dimethylamide (100 mg, 0.22 mmol; see Example 19, subpart (a) above) and tetrakis-(triphenylphosphino)palladium (25 mg, 0.022 mmol). The mixture was heated to 120° C. for 2 hours and allowed to cool to room temperature for overnight with stirring. The resulting reaction mixture was diluted with EtOAc and washed with saturated NH₄Cl. The orgainc layer was dried over MgSO₄ and concentrated. The residue was purified on silica gel column with 5% EtOAc/CH₂Cl₂ to produce 4-benzo[1,3]dioxol-5-yl-3-(6-cyclopropyl-pyridin-2-yl)-pyrazole-1-sulfonic acid dimethylamide (51 mg, 56%). 4-Benzo[1,3]dioxol-5-yl-3-(6-cyclopropyl-pyridin-2-yl)-pyrazole-1-sulfonic acid dimethylamide (50 mg, 0.12 mmol) was then dissolved in a mixture of THF (1 mL) and EtOH (4 mL) and a solution of NaOEt in EtOH (23%, 1 mL) was added. The resulting solution was then heated to reflux for 2 hours and allowed to cool to room temperature and concentrated. The residue was filtered through a short silica gel cake and washed with THF. The filtrate was concentrated and redissolved in DMSO for purification by semi-preparative HPLC to produce the title compound (10 mg, 27%). MS (ESP+) m/z 306.3 (M+1). ¹H NMR (300 MHz, MeOH-d₄, δ): 8.23 (t, 1H), 7.95 (s, 1H), 7.55 (d, 1H), 7.41 (d, 1H), 6.90-6.81 (m, 3H), 6.01 (s, 2H), 2.6-2.5 (m, 1H), 1.55-1.41 (m, 2H), 1.27-1.22 (m, 2H).

### EXAMPLE 22

### 2-(4-Benzo[1,3]dioxol-5-yl-1H-pyrazol-3-yl)-6-trifluoromethyl-pyridine

A solution of 4-benzo[1,3]dioxol-5-yl-3-(6-bromo-pyridin-2-yl)-pyrazole-1-sulfonic acid dimethylamide (170 mg, 0.37 mmol; see Example 19, subpart (a) above) and methyl fluorosulfonyldifluoroacetate (362 mg, 1.87 mmol) in anhydrous DMF (4 mL) was flushed with nitrogen gas 3 times. Copper powder (12 mg, 0.19 mmol) was then added to the reaction mixture, which was heated to 80° C. for 4 hours. It was cooled down to room temperature and extracted with diethyl ether and water. The ether extract was washed with EDTA (0.5 M, 20 mL) twice and water once, then dried over MgSO₄ and concentrated to give crude 4-benzo[1,3]dioxol-5-yl-3-(6-trifluoromethyl-pyridin-2-yl)-pyrazole-1-sulfonic acid dimethylamide (160 mg) as a bright yellow foam. The crude produce was then dissolved in EtOH (10 mL) and a solution of NaOEt in EtOH (23%, 1 mL) was added. The reaction mixture was then heated to reflux for overnight, cooled to room temperature, and concentrated. The residue was filtered through a short silica gel cake and washed with THF. The filtrate was concentrated and redissolved in DMSO and purified by semi-preparative HPLC to produce the title compound (65 mg, 52% for 2 steps). MS (ESP+) m/z 334.2 (M+1).). ¹H NMR (300 MHz, MeOH-d₄, δ): 7.94 (t, 1H), 7.73-7.69 (m, 3H), 6.87-6.74 (m, 3H), 5.95 (s, 2H).

### EXAMPLE 23

### 4-(1-Oxo-1,2-dihydro-isoquinolin-4-yl)-3-pyridin-2-yl-pyrazole-1-sulfonic acid dimethylamide

To a solution of 55 mg (0.13 mmol) of (1-methoxy-isoquinolin-4-yl)-3-pyridin-2-yl-pyrazole-1-sulfonic acid dimethylamide (which is prepared by coupling 4-Bromo-1-methoxyisoquinoline (the title compound of Example I above) with 1-(N,N-dimethyl)-sulfamoyl-3-pyridin-2-yl-pyrazole-4-boronic acid (the title compound of Example 1(d) above) in the same manner as described in Example 1(e) above) in 5 mL dry acetonitrile was added 0.37 mL (2.6 mmol, 20 equiv.) iodotrimethylsilane to give an orange solution. The reaction mixture was heated to 70° C. with stifling overnight, which was allowed to cool to room temperature, diluted with ethyl acetate, and washed with 10% aq. sodium thiosulfate, water, and brine. The resulting solution was then dried (Na2SO4), filtered, and concentrated to the title compound as a yellow solid without further purification; m/z 396 [M+H]+.

### EXAMPLE 24

### 2-(4-benzo[1,3]dioxol-5-yl-5-trifluoromethyl-1H-pyrazol-3-yl)-6-bromo-pyridine

A solution of 2-benzo[1,3]dioxol-5-yl-1-(6-bromo-pyridin-2-yl)-ethanone (0.359 mmol) in anhydrous THF (5 mL) was added to a slurry of sodium hydride (0.725 mmol) in anhydrous THF (5 mL) at RT. After 5 minutes, N-trifluoroacetylimidazole (0.395 mmol) was added. After an additional 30 minutes at room temperature, hydrazine (1.5 mL) was added. After another 30 minutes, glacial acetic acid (10 mL) was added and the reaction warmed to 100° C. for 1 hour. The reaction was then concentrated in vacuo and purified via reverse phase HPLC (acetonitrile-water gradient) to give a solid identified as 2-(4-benzo[1,3]dioxol-5-yl-5-trifluoromethyl-1H-pyrazol-3-yl)-6-bromo-pyridine: MS (ESP+) 411.9 (M+1).

### EXAMPLE 25

### 1-tert-Butyl-3-[6-(3-pyridin-2-yl-1H-pyrazol-4-yl)-quinazolin-4-yl]-urea

To an oven-dried 100 mL round bottom flask was added 500 mg (1.26 mmol) of 4-(4-amino-quinazolin-6-yl)-3-pyridin-2-yl-pyrazole-1-sulfonic acid dimethylamide (which is prepared by coupling 6-iodo-4-aminoquinazoline (the title compound of Example G above) with 1-(N,N-dimethyl)-sulfamoyl-3-pyridin-2-yl-pyrazole-4-boronic acid (the title compound of Example 1(d) above) in the same manner as described in Example 1(e) above), the flask capped with a rubber septum and flushed with argon. To this was added 15 mL dry DMF with stirring to give a colorless solution, then 60 mg (1.5 mmol, 1.2 equiv.) of NaH (60% w/w in mineral oil) was added, giving copious gas evolution and producing a yellow mixture. This was stirred at ambient temperature for 30 min., then 145 μL (1.26 mmol) of tert-butyl isocyanate was added and the resulting mixture stirred overnight. The yellow reaction was quenched with about 0.5 mL glacial AcOH and the colorless solution concentrated. The residue was treated with H₂O to give a light brown solid. This was filtered off, washed with water, and air-dried, then recrystallized from ethanol/water to give 585 mg (1.18 mmol, 94%) of 4-[4-(3-tert-butyl-ureido)-quinazolin-6-yl]-3-pyridin-2-yl-pyrazole-1-sulfonic acid dimethylamide as a tan solid. X-ray diffraction-quality crystals were obtained from chloroform/hexane. ¹H-NMR (300 MHz, DMSO-d₆, δ): 9.95 (1H; s), 9.93 (1H; s), 8.83 (1H; s), 8.73 (1H; s), 8.70 (1H; s), 8.50 (1H; d, J=4 Hz), 7.93 (1H; t, J=4 Hz), 7.88 (1H; d, J=8 Hz), 7.71 (2H; m), 7.45 (1H; m), 2.94 (6H; s), 1.36 (9H; s); m/z 495 [M+H]⁺.

4-[4-(3-tert-Butyl-ureido)-quinazolin-6-yl]-3-pyridin-2-yl-pyrazole-1-sulfonic acid dimethylamide was then deprotected in the same manner as described in Example 2 to produce the title compound. ¹H-NMR (400 MHz, CDCl₃, δ): 10.19 (s, 1H), 9.86 (s, 1H), 8.81 (s, 1H), 8.60 (br s, 2H), 7.93 (s, 2H), 7.66 (s, 1H), 7.60 (t, J=8 Hz, 1H), 7.43 (d, J=8 Hz, 1H), 7.20 (m, 1H), 1.54 (s, 9H); m/z 388 [M+H]⁺.

### EXAMPLE 26

### 4-Morpholin-4-yl-6-(3-pyridin-2-yl-1H-pyrazol-4-yl)-quinazoline

To a solution of 0.2 gram (0.48 mmol) of 4-(4-chloro-quinazolin-6-yl)-3-pyridin-2-yl-pyrazole-1-sulfonic acid dimethylamide (which is prepared by coupling 4-chloro-6-iodo-quinazoline (Davos Chemical Corp., Upper Saddle River, N.J.) with 1-(N,N-dimethyl)-sulfamoyl-3-pyridin-2-yl-pyrazole-4-boronic acid (the title compound of Example 1(d) above) in the same manner as described in Example 1(e) above) in 4 mL acetonitrile in a high-pressure tube was added 0.13 mL (1.5 mmol) morpholine to give a colorless solution. The tube was capped and the solution heated to 85° C. with stirring for three hours. The resulting solution was cooled, concentrated, and the residue brought up in ethyl acetate. This was washed with a 5% citric acid solution, then brine, and dried (Na₂SO₄), filtered and concentrated to form 4-(4-morpholin-4-yl-quinazolin-6-yl)-3-pyridin-2-yl-pyrazole-1-sulfonic acid dimethylamide (0.14 gram, 0.39 mmol, 70%), which was used in the next step without further purification; m/z: 466 (M+1)⁺.

4-(4-Morpholin-4-yl-quinazolin-6-yl)-3-pyridin-2-yl-pyrazole-1-sulfonic acid dimethylamide was then deprotected in the same manner as described in Example 2 to produce the title compound. ¹H-NMR (300 MHz, CDCl₃, δ): 8.79 (s, 1H), 8.67 (d, J=4 Hz, 1H), 7.99 (d, J=9 Hz, 1H), 7.89 (s, 1H), 7.84 (dd, J=2 Hz, 9 Hz, 1H), 7.76 (s, 1”), 7.60 (td, J=2 Hz, 8 Hz, 1H), 7.40 (d, J=8 Hz, 1H), 7.27 (m, 1H), 3.78 (m, 4H), 3.73 (m, 4H); m/z 359 [M+H]⁺.

### EXAMPLE 27

### 4-(4-Methoxy-phenyl)-6-(3-pyridin-2-yl-1H-pyrazol-4-yl)-quinazoline

To a solution of 180 mg (0.43 mmol) of 4-(4-chloro-quinazolin-6-yl)-3-pyridin-2-yl-pyrazole-1-sulfonic acid dimethylamide (which is prepared by coupling 4-chloro-6-iodo-quinazoline (Davos Chemical Corp., Upper Saddle River, N.J.) with 1-(N,N-dimethyl)-sulfamoyl-3-pyridin-2-yl-pyrazole-4-boronic acid (the title compound of Example 1 (d) above) in the same manner as described in Example 1 (e) above) in 5 mL toluene in a high-pressure tube was added 99 mg (0.65 mmol, 1.5 equiv.) 4-methoxybenzeneboronic acid, 90 mg (0.65 mmol) solid K₂CO₃, and 25 mg (0.022 mmol, 5 mol %) tetrakis(triphenyl-phosphine)palladium (0) to give a yellow solution. The tube was flushed with argon, capped and the solution heated to 100° C. with stirring overnight. The resulting mixture was cooled, diluted with ethyl acetate, washed with 1N NaOH, a 5% citric acid solution, then brine, and dried (Na₂SO₄), filtered and concentrated to form 4-[4-(4-methoxy-phenyl)-quinazolin-6-yl]-3-pyridin-2-yl-pyrazole-1-sulfonic acid dimethylamide (160 mg, 0.33 mmol, 77%) which was used in the next step without further purification; m/z: 487 (M+1)⁺.

4-[4-(4-Methoxy-phenyl)-quinazolin-6-yl]-3-pyridin-2-yl-pyrazole-1-sulfonic acid dimethylamide was then deprotected in the same manner as described in Example 2 to produce the title compound. ¹H-NMR (300 MHz, DMSO-d₆, δ): 8.84 (d, J=1 Hz, 1H), 8.67 (dd, J=2 Hz, 4 Hz, 1H), 8.25 (d, J=2 Hz, 1H), 7.98 (d, J=8 Hz, 2H), 7.92 (d, J=9 Hz, 1H), 7.86 (dt, J=2 Hz, 9 Hz, 1H), 7.76 (d, J=1 Hz, 1H), 7.55 (tt, J=2 Hz, 8 Hz, 1H), 7.32 (d, J=8 Hz, 1H), 7.23 (m, 1H), 7.11 (d, J=8 Hz, 2H), 4.05 (s, 3H); m/z: 380 (M+1)⁺.

### EXAMPLE 28

### 5-Methyl-thiophene-2-carboxylic acid [6-(3-pyridin-2-yl-1H-pyrazol-4-yl)-quinazolin-4-yl]-amide

To a solution of 200 mg (0.5 mmol) of 4-(4-amino-quinazolin-6-yl)-3-pyridin-2-yl-pyrazole-1-sulfonic acid dimethylamide (which is prepared by coupling 6-iodo-4-aminoquinazoline (the title compound of Example G above) with 1-(N,N-dimethyl)-sulfamoyl-3-pyridin-2-yl-pyrazole-4-boronic acid (the title compound of Example 1 (d) above) in the same manner as described in Example 1(e) above) in 10 mL CH₃CN was added 0.28 mL (2.0 mmol) triethylamine, then 97 mg (0.6 mmol) 5-methylthiophene-2-carbonyl chloride (Oakwood Products, Inc., West Columbia, S.C.) with stirring to give a yellow solution. This was heated to reflux overnight, then cooled, diluted with ethyl acetate, washed with 1N NaOH, then a 5% solution of citric acid, then brine. The organic phase was dried, filtered and concentrated to form 5-methyl-thiophene-2-carboxylic acid [6-(1-dimethylsulfamoyl-3-pyridin-2-yl-1H-pyrazol-4-yl)-quinazolin-4-yl]-amide, a yellow solid, which was used in the next step without further purification; m/z: 520 [M+H]⁺.

5-Methyl-thiophene-2-carboxylic acid (6-(1-dimethylsulfamoyl-3-pyridin-2-yl-1H-pyrazol-4-yl)-quinazolin-4-yl]-amide was then deprotected in the same manner as described in Example 2 to produce the title compound. ¹H-NMR (300 MHz, DMSO-d₆, δ): 8.56 (s, 1H), 8.67 (dd, J=2 Hz, 4 Hz, 1H), 8.25 (d, J=2 Hz, 1H), 8.15 (s, 1H), 8.01 (m, 1H), 7.99 (m, 1H), 7.92 (d, J-=8 Hz, 1H), 7.86 (d, J=8 Hz, 1H), 7.76 (d, J=2 Hz, 1H), 7.55 (t, J=8 Hz, 1H), 7.40 (d, J=8 Hz, 1H), 6.90 (m, 1H), 2.50 (s, 3H); m/z: 413 [M+H]⁺.

### EXAMPLE 29

### (4-Methoxy-phenyl)-[6-(3-pyridin-2-yl-1H-pyrazol-4-yl)-quinazolin-4-yl]-methanone

To a stirred solution of 500 mg (1.2 mmol) 4-(4-amino-quinazolin-6-yl)-3-pyridin-2-yl-pyrazole-1-sulfonic acid dimethylamide (which is prepared by coupling 6-iodo-4-aminoquinazoline (the title compound of Example G above) with 1-(N,N-dimethyl)-sulfamoyl-3-pyridin-2-yl-pyrazole-4-boronic acid (the title compound of Example 1(d) above) in the same manner as described in Example 1(e) above), 0.16 mL (1.3 mmol) p-anisaldehyde, and 53 mg (0.4 mmol) 1,3-dimethylimidazolium methanesulfonate (Fluka) in dioxane under argon was added 53 mg (1.3 mmol) of a 60% dispersion of sodium hydride in oil to give a yellow mixture. This mixture was heated to reflux overnight, then cooled, poured onto ice-water, and extracted with ethyl acetate. The organic layer was washed with 1N NaOH, a 5% solution of citric acid, then brine, and dried (Na₂SO₄). Filtration and evaporation gave a yellow residue, which was recrystallized from ethanol/water to give 276 mg (0.5 mmol, 45%) of 4-[4-(4-methoxy-benzoyl)-quinazolin-6-yl]-3-pyridin-2-yl-pyrazole-1-sulfonic acid dimethylamide as fine, pale yellow crystals; m/z: 516 [M+1]⁺.

4-[4-(4-Methoxy-benzoyl)-quinazolin-6-yl]-3-pyridin-2-yl-pyrazole-1-sulfonic acid dimethylamide was then deprotected in the same manner as described in Example 2 to produce the title compound. ¹H-NMR (300 MHz, DMSO-d₆, δ): 8.86 (d, J=2 Hz, 1H), 8.64 (d, J=4 Hz, 1H), 8.29 (d, J=2 Hz, 1H), 8.10 (d, J=8 Hz, 2H), 7.96 (d, J=9 Hz, 1H), 7.82 (m, 1H), 7.72 (d, J=1 Hz, 1H), 7.51 (tt, J=2 Hz, 8 Hz, 1H), 7.30 (d, J=8 Hz, 1H), 7.19 (m, 1H), 7.13 (d, J=8 Hz, 2H), 4.15 (s, 3H); m/z: 408 [M+1]⁺.

The compounds listed in the following table were prepared in an analogous manner as described in the methods and examples above. The NMR and mass spectroscopy data of these compounds are included in the table (note that “n/a” indicates that NMR data are not available for that compound).

| | | | | Synthetic |
|---|---|---|---|---|
| Example | Compound Name | ¹H-NMR | Mass Spec. (m/z) | Method |
| Ex. 30 | N-[3-(3-Pyridin-2-yl-4- | ¹H-NMR(CDCl₃, 300MHz, δ) 8.86(s, 1H), | 372[M+H]+ | Ex. 10, 11, |
| | quinolin-4-yl-pyrazol-1- | 8.46(s, 1H), 8.11(d, J=9Hz, 1H), | | and 13 |
| | yl)-propyl]-acetamide | 7.76(d, J=8Hz, 1H), 7.64(t, J=8Hz, 2H), | | |
| | | 7.42(t, J=8Hz, 1H), 7.34(t, J=7Hz, | | |
| | | 1H), 7.29(d, J=6Hz, 1H), 7.13(s, 1H), | | |
| | | 6.17(s, 1H), 4.37(t, J=7Hz, 2H), 3.39(t, | | |
| | | J=7Hz, 2H), 2.22(q, J=2Hz, 7Hz, | | |
| | | 2H), 1.94(s, 3H). | | |
| Ex. 31 | N-[3-(3-Pyridin-2-yl-4- | ¹H-NMR(300MHz, CDCl₃, δ): 8.88(s, | 408[M+H]+ | Ex. 10, 11, |
| | quinolin-4-yl-pyrazol-1- | 1H), 8.42(s, 1H), 8.14(d, J=5Hz, 1H), | | and 13 |
| | yl)-propyl]- | 7.78(d, J=8Hz, 1H), 7.67(t, J=7Hz, | | |
| | methanesulfonamide | 2H), 7.46(m, 1H), 7.36(m, 3H), 7.13(s, | | |
| | | 1H), 5.20(s, 1H), 4.45(t, J=7Hz, 2H), | | |
| | | 3.29(t, J=7Hz, 2H), 2.97(s, 3H), | | |
| | | 2.30(q, J=2Hz, 7Hz, 2H). | | |
| Ex. 32 | 4-{3-Pyridin-2-yl-1-[2- | ¹H-NMR(300MHz, DMSO-d₆, δ): | 369[M+H]+. | Ex. 10, 11, |
| | (1H-tetrazol-5-yl)-ethyl]- | 8.79(d, J=5Hz, 1H), 8.09(d, J=4Hz, 1H), | | and 16 |
| | 1H-pyrazol-4-yl}- | 8.03(s, 1H), 7.99(d, J=8Hz, 1H), | | |
| | quinoline | 7.82(d, J=8Hz, 1H), 7.75(td, J=2Hz, 8Hz, | | |
| | | 1H), 7.69(m, 1H), 7.39(td, J=1Hz, 8Hz, | | |
| | | 1H), 7.28(d, J=4Hz, 1H), 7.13(t, J=5Hz, | | |
| | | 1H), 4.56(t, J=7Hz, 2H), 3.31(t, J=7Hz, | | |
| | | 2H); | | |
| Ex. 33 | 2-[4-(4-Methoxy- | ¹H-NMR(300MHz, DMSO-d₆, δ): | 252[M+H]+ | Ex. 1 and 2 |
| | phenyl)-1H-pyrazol-3- | 11.50(br s, 1H), 8.54(d, J=4Hz, 1H), 8.24(s, | | |
| | yl]-pyridine | 1H); 7.89(tt, J=2Hz, 8Hz, 1H), 7.70(d, | | |
| | | J=7Hz, 1H), 7.41(t, J=6Hz, 1H), | | |
| | | 7.28(dd, J=1Hz, 5Hz, 2H), 6.85(dd, J=1Hz, | | |
| | | 5Hz, 2H), 3.74(s, 3H). | | |
| Ex. 34 | 2-Chloro-5-(3-pyridin-2- | ¹H-NMR(300MHz, DMSO-d₆, δ): 13.3(br | 257.7[M+H]+ | Ex. 1 and 2 |
| | yl-1H-pyrazol-4-yl)- | s, 1H), 8.70(t, J=4Hz, 1H), 8.36(t, J=4Hz, | | |
| | pyridine | 1H), 8.08(s, 1H), 7.80(m, 3H), | | |
| | | 7.39(d, J=8Hz, 1H), 7.27(s, 1H). | | |
| Ex. 35 | 5-(3-Pyridin-2-yl-1H- | ¹H-NMR(300MHz, CDCl₃, δ): 11.35(br s, | 238[M+H]+ | Ex. 1 and 2 |
| | pyrazol-4-yl)-pyridin-2- | 1H), 8.61(d, J=4Hz, 1H), 8.15(d, J=2Hz, | | |
| | ylamine | 1H), 7.73(t, J=6Hz, 1H), 7.60(s, | | |
| | | 1H), 7.50(dd, J=2Hz, 8Hz, 1H), 7.39(d, | | |
| | | J=8Hz, 1H), 7.21(m, 1H), 6.58(d, J=8Hz, | | |
| | | 1H), 4.60(br s, 2H). | | |
| Ex. 36 | 2,4-Dimethoxy-5-(3- | ¹H-NMR(300MHz, DMSO-d₆, δ): 11.31(br | 284[M+H]+ | Ex. 1 and 2 |
| | pyridin-2-yl-1H-pyrazol- | s, 1H), 8.43(d, J=4Hz, 1H), 8.20(t, J=4Hz, | | |
| | 4-yl)-pyrimidine | 1H), 7.80(m, 2H), 7.64(d, J=4Hz, | | |
| | | 1H), 7.26(t, J=4Hz, 1H), 3.91(s, | | |
| | | 3H), 3.62(s, 3H). | | |
| Ex. 37 | 2-[4-(3,4-Dimethoxy- | ¹H-NMR(300MHz, CDCl₃, δ): 11.35(br s, | 282[M+H]+ | Ex. 1 and 2 |
| | phenyl)-1H-pyrazol-3- | 1H), 8.65(d, J=4Hz, 1H), 7.66(s, 1H), | | |
| | yl]-pyridine | 7.60(t, J=7Hz, 1H), 7.41(d, J=8Hz, | | |
| | | 1H), 7.28(m, 1H), 6.95(m, 3H), 3.94(s, | | |
| | | 3H), 3.83(s, 3H). | | |
| Ex. 38 | 5-(3-Pyridin-2-yl-1H- | ¹H-NMR(300MHz, CDCl₃, δ): 8.60(d, J=4Hz, | 261[M+H]+ | Ex. 1 and 2 |
| | pyrazol-4-yl)-1H-indole | 1H), 8.30(br s, 1H), 7.71(s, 1H), | | |
| | | 7.67(q, J=1Hz, 4Hz, 2H), 7.44(d, J=8Hz, | | |
| | | 2H), 7.28(m, 1H), 7.16(m, 1H), | | |
| | | 6.58(d, J=4Hz, 1H). | | |
| Ex. 39 | 2-[4-(3-Methoxy- | ¹H-NMR(300MHz, CDCl₃, δ): 11.53(br s, | 252[M+H]+ | Ex. 1 and 2 |
| | phenyl)-1H-pyrazol-3- | 1H), 8.64(d, J=4Hz, 1H), 7.66(s, 1H), | | |
| | yl]-pyridine | 7.56(td, J=2Hz, 8Hz, 1H), 7.41(d, J=8Hz, | | |
| | | 1H), 7.35(t, J=8Hz, 1H), 7.20(td, | | |
| | | J=1Hz, 7Hz, 1H), 7.02(d, J=8Hz, | | |
| | | 1H), 6.97(t, J=2Hz, 1H), 6.91(dd, J=2Hz, | | |
| | | 8Hz, 1H), 3.73(s, 3H). | | |
| Ex. 40 | 2-[4-(2,3-Dihydro- | ¹H-NMR(300MHz, DMSO-d₆, δ): | 280[M+H]+ | Ex. 1 and 2 |
| | benzo[1,4]dioxin-6-yl)- | 13.17(br s, 1H), 8.57(s, 1H), 7.78(m, 2H), | | |
| | 1H-pyrazol-3-yl]- | 7.54(m, 1H), 7.32(t, J=6Hz, 1H), 6.84(t, J=1Hz, | | |
| | pyridine | 1H), 6.78(m, 2H), 4.22(s, 4H). | | |
| Ex. 41 | 2-(3-Pyridin-2-yl-4- | ¹H-NMR(300MHz, DMSO-d₆, δ): 9.20(d, | 316[M+H]+ | Ex. 6 and 11 |
| | quinolin-4-yl-pyrazol-1- | J=4Hz, 1H), 8.46(s, 1H), 8.39(d, J=8Hz, | | |
| | yl)-ethylamine | 1H), 8.17(br s, 2H), 8.10(d, J=5Hz, | | |
| | | 1H), 8.06(t, J=7Hz, 1H), 8.01(d, J=8Hz, | | |
| | | 1H), 7.87(m, 3H), 7.72(t, J=7Hz, | | |
| | | 1H), 7.26(t, J=6Hz, 1H), 4.66(t, J=7Hz, | | |
| | | 2H), 3.45(t, J=7Hz, 2H). | | |
| Ex. 42 | N-[2-(3-Pyridin-2-yl-4- | ¹H-NMR(300MHz, CDCl₃, δ): 8.88(s, | 394[M+H]+ | Ex. 6, 11, |
| | quinolin-4-yl-pyrazol-1- | 1H), 8.42(s, 1H), 8.14(d, J=5Hz, 1H), | | and 13 |
| | yl)-ethyl]- | 7.78(d, J=8Hz, 1H), 7.67(t, J=7Hz, | | |
| | methanesulfonamide | 2H), 7.46(m, 1H), 7.36(m, 3H), 7.13(s, | | |
| | | 1H), 5.20(s, 1H), 4.60(t, J=7Hz, 2H), | | |
| | | 3.55(t, J=7Hz, 2H), 2.94(s, 3H). | | |
| Ex. 43 | 2-Methyl-4- | ¹H-NMR(300MHz, CDCl₃, δ): 8.65(d, J=4Hz, | 284[M+H]+ | Ex. 1 and 2 |
| | methylsulfanyl-6-(3- | 1H), 8.13(d, J=8Hz, 1H), 8.04(s, | | |
| | pyridin-2-yl-1H-pyrazol- | 1H), 7.74(td, J=2Hz, 8Hz, 1H), 7.32(q, | | |
| | 4-yl)-pyrimidine | J=5Hz, 8Hz, 1H), 2.47(s, 6H). | | |
| Ex. 44 | 3-(3-Pyridin-2-yl-1H- | ¹H-NMR(300MHz, CDCl₃, δ): 8.67(d, J=4Hz, | 247[M+H]+ | Ex. 1 and 2 |
| | pyrazol-4-yl)- | 1H), 7.68(m, 5H), 7.51(t, J=8Hz, | | |
| | benzonitrile | 1H), 7.29(m, 2H). | | |
| Ex. 45 | 3-(3-Pyridin-2-yl-1H- | ¹H-NMR(300MHz, DMSO-d₆, δ): | 266[M+H]+ | Ex. 1 and 2 |
| | pyrazol-4-yl)-benzoic | 12.87(br s, 1H), 8.46(br s, 1H), 7.86(t, J=4Hz, | | |
| | acid | 2H), 7.73(m, 3H), 7.52(dd, J=2Hz, | | |
| | | 6Hz, 1H), 7.33(m, 1H), 7.22(t, J=4Hz, | | |
| | | 1H); | | |
| Ex. 46 | 2-(4-Benzo[1,3]dioxol-5- | ¹H-NMR(300MHz, CDCl₃, δ): 12.59(br s, | 266[M+H}+ | Ex. 1 and 2 |
| | yl-1H-pyrazol-3-yl)- | 1H), 8.62(s, 1H), 7.80(m, 2H), 7.55(m, | | |
| | pyridine | 1H), 7.37(t, J=6Hz, 1H), 6.88(t, J=2Hz, | | |
| | | 1H), 6.78(m, 2H), 6.02(s, 2H). | | |
| Ex. 47 | 2-[4-(2,3-Dihydro- | ¹H-NMR(300MHz, CDCl₃, δ): 12.59(br s, | 264[M+H]+ | Ex. 1 and 2 |
| | benzofuran-5-yl)-1H- | 1H), 8.65(s, 1H), 7.82(m, 2H), 7.50(m, | | |
| | pyrazol-3-yl]-pyridine | 1H), 7.31(t, J=6Hz, 1H), 6.88(t, J=2Hz, | | |
| | | 1H), 6.78(m, 2H), 4.63(t, J=8Hz, | | |
| | | 2H), 3.22(t, J=8Hz, 2H). | | |
| Ex. 48 | 5-(3-Pyridin-2-yl-1H | ¹H-NMR(300MHz, DMSO-d₆, δ): | 263[M+H]+ | Ex. 1 and 2 |
| | pyrazol-4-yl)- | 11.03(br s, 1H), 8.54(br s, 1H), 7.76(m, 4H), | | |
| | benzo[d]isoxazole | 7.60(t, J=2Hz, 1H), 7.50(d, J=9Hz, | | |
| | | 1H), 7.33(m, 1H), 6.94(m, 1H). | | |
| Ex. 49 | 3-[4-Benzo[1,3]dioxol-5- | Data for free base: ¹H-NMR(400MHz, | 333[M+H]+. | Ex. 10 and |
| | yl-3-(6-methyl-pyridin-2- | DMSO-d₆, δ): 8.04(s, 1H), 7.61(t, J=12Hz, | | 11 |
| | yl)-pyrazol-1-yl]- | 1H), 7.39(d, J=6Hz, 1H), 7.20(d, J=6Hz, | | |
| | propionitrile | 1H), 7.05(s, 1H), 6.83(s, 2H), | | |
| | | 5.97(s, 2H), 4.44(t, J=6Hz, 2H), 3.13(t, | | |
| | | J=6Hz, 2H), 2.96(s, 3H). | | |
| | | Regiochemistry assigned by 2D-NMR. | | |
| Ex. 50 | N-{3-[4- | ¹H-NMR(300MHz, CDCl₃, δ): 7.97(d, J=4Hz, | 415[M+H]+. | Ex. 10, 11, |
| | Benzo[1,3]dioxol-5-yl-3- | 1H), 7.55(s, 1H), 7.40(m, 2H), | | and 13 |
| | (6-methyl-pyridin-2-yl)- | 7.13(s, 1H), 6.79(d, J=8Hz, 2H), | | |
| | pyrazol-1-yl]-propyl}- | 6.00(s, 2H), 4.46(t, J=6Hz, 2H), 3.20(m, | | |
| | methanesulfonamide | 5H), 2.96(s, 3H), 2.36(t, J=6Hz, 2H) | | |
| Ex. 51 | 2-[4-(2,3-Dihydro- | ¹H-NMR(DMSO-d₆, 400MHz, δ) 8.24(t, | 294[M+H]+ | Ex. 5 |
| | benzol[1,4]dioxin-6-yl)- | J=8Hz, 1H), 8.10(s, 1H), 7.72(d, J=8Hz, | | |
| | 1H-pyrazol-3-yl]-6- | 1H), 7.50(d, J=8Hz, 1H), 6.89(d, J=2Hz, | | |
| | methyl-pyridine | 1H), 6.83(d, J=8Hz, 1H), | | |
| | | 6.76(dd, J=2Hz, 8Hz, 1H), 4.24(s, 4H), | | |
| | | 2.73(s, 3H). | | |
| Ex. 52 | [4-Benzo[1,3]dioxol-5-yl- | ¹H-NMR(300MHz, CDCl₃, δ): 7.61(s, | 319[M+H]+ | Ex. 6 |
| | 3-(6-methyl-pyridin-2- | 1H), 7.50(q, J=6Hz, 15Hz, 1H), | | |
| | yl)-pyrazol-1-yl]- | 7.11(m, 2H), 6.77(s, 1H), 6.75(s, 2H), 5.95(s, | | |
| | acetonitrile | 2H), 5.18(s, 2H), 2.60(s, 3H); m/z: | | |
| | | 319[M+H]⁺. Regiochemistry assigned by 2D- | | |
| | | NMR. | | |
| Ex. 53 | N-{2-[4- | ¹H-NMR(300MHz, CDCl₃, δ): 7.97(d, J=4Hz, | 401[M+H]+ | Ex. 6, 11, |
| | Benzo[1,3]dioxol-5-yl-3- | 1H), 7.55(s, 1H), 7.40(m, 2H), | | and 13 |
| | (6-methyl-pyridin-2-yl)- | 7.13(s, 1H), 6.79(d, J=8Hz, 2H), | | |
| | pyrazol-1-yl]-ethyl}- | 6.00(s, 2H), 4.56(t, J=6Hz, 2H), 3.45(t, J=6Hz, | | |
| | methanesulfonamide | 2H), 3.05(s, 3H). | | |
| Ex. 54 | 4-[3-(6-Methyl-pyridin-2- | n/a | 284[M+H]+ | Ex. 5 |
| | yl)-1H-pyrazol-4-yl]-2- | | | |
| | methylsulfanyl- | | | |
| | pyrimidine | | | |
| Ex. 55 | 4-(3-Pyridin-2-yl-1H- | ¹H-NMR(300MHz, DMSO-d₆, δ): | 290[M+H]+ | Ex. 2 and 4 |
| | pyrazol-4-yl)-2H- | 12.68(s, 1H), 8.35(d, J=4Hz, 1H), 8.28(d, J=7Hz, | | |
| | phthalazin-1-one | 1H), 8.12(s, 1H), 7.90(d, J=8Hz, | | |
| | | 1H), 7.78(m, 1H), 7.71(m, 3H), 7.35(d, J=8Hz, | | |
| | | 1H), 7.24(t, J=6Hz, 1H). | | |
| Ex. 56 | 1-[5-(3-Pyridin-2-yl-1H- | ¹H-NMR(300MHz, CDCl₃, δ): 11.20(br s, | 305[M+H]+ | Ex. 1 and 2 |
| | pyrazol-4-yl)-2,3- | 1H), 8.57(d, J=4Hz, 1H), 8.09(d, J=8Hz, | | |
| | dihydro-indol-1-yl]- | 1H), 8.05(s, 1H), 7.67(m, 1H), | | |
| | ethanone | 7.56(dd, J=2Hz, 8Hz, 1H), 7.28(m, 1H), | | |
| | | 7.18(s, 1H), 7.09(d, J=4Hz, 1H), | | |
| | | 4.10(t, J=8Hz, 2H), 3.17(t, J=8Hz, 2H), | | |
| | | 2.23(s, 2H). | | |
| Ex. 57 | 6-(3-Pyridin-2-yl-1H- | ¹H-NMR(300MHz, CDCl₃, δ): 11.12(br | 263[M+H]+ | Ex. B, 1, and 2 |
| | pyrazol-4-yl)- | s, 1H), 8.73(q, J=1Hz, 2Hz, 1H), | | |
| | [1,2,4]triazolo[1,5- | 8.63(dd, J=1Hz, 5Hz, 1H), 8.39(s, 1H), | | |
| | a]pyridine | 7.80(dd, J=1Hz, 9Hz, 1H), 7.73(s, 1H), | | |
| | | 7.61(qd, J=2Hz, 9Hz, 16Hz, 2H), 7.39(d, J=8Hz, | | |
| | | 1H), 7.24(m, 1H). | | |
| Ex. 58 | 3-Methyl-6-(3-pyridin-2- | ¹H-NMR(300MHz, CDCl₃, δ): 11.24(br s, | 304[M+H]+ | Ex. A, 1, and 2 |
| | yl-1H-pyrazol-4-yl)-3H- | 1H), 8.63(d, J=5Hz, 1H), 8.41(d, J=2Hz, | | |
| | quinazolin-4-one | 1H), 8.08(s, 1H), 7.80(dd, J=2Hz, 8Hz, | | |
| | | 1H), 7.74(q, J=3Hz, 8Hz, 2H), | | |
| | | 7.54(td, J=2Hz, 8Hz, 1H), 7.34(d, J=8Hz, | | |
| | | 1H), 7.24(m, 1H), 3.62(s, 3H). | | |
| Ex. 59 | 6-(3-Pyridin-2-yl-1H- | n/a | 293[M+H]+ | Ex. 1 and 2 |
| | pyrazol-4-yl)-4H- | | | |
| | benzo[1,4]oxazin-3-one | | | |
| Ex. 60 | 6-(3-Pyridin-2-yl-1H- | ¹H-NMR(300MHz, CDCl₃, δ): 11.50(br | 274[M+H]+ | Ex. 1 and 2 |
| | pyrazol-4-yl)- | s, 1H), 8.87(d, J=1Hz, 2H), 8.67(d, J=5Hz, | | |
| | quinoxaline | 1H), 8.21(d, J=2Hz, 1H), 8.14(d, | | |
| | | J=9Hz, 1H), 7.84(dd, J=2Hz, 9Hz, | | |
| | | 1H), 7.82(s 1H), 7.56(td, J=1Hz, 7Hz, | | |
| | | 1H), 7.40(d, J=8Hz, 1H), 7.25(m, 1H). | | |
| Ex. 61 | 3-(4-Nitro-benzyl)-6-(3- | ¹H-NMR(300MHz, CDCl₃, δ): 8.62(d, J=5Hz, | 425[M+H]+ | Ex. 1 and 2 |
| | pyridin-2-yl-1H-pyrazol- | 1H), 8.40(d, J=2Hz, 1H), 8.23(d, | | |
| | 4-yl)-3H-quinazolin-4- | J=8Hz, 2H), 8.15(s, 1H), 7.78(m, 3H), | | |
| | one | 7.56(t, J=9Hz, 3H), 7.32(m, 2H), | | |
| | | 5.30(s, 2H). | | |
| Ex. 62 | 5-Methyl-6-(3-pyridin-2- | ¹H-NMR(300MHz, CDCl₃, δ): 11.35(br s, | 277[M+H]+ | Ex. B, 1, and 2 |
| | yl-1H-pyrazol-4-yl)- | 1H), 8.62(d, J=4Hz, 1H), 8.43(s, 1H), | | |
| | [1,2,4]triazolo[1,5- | 7.73(d, J=8Hz, 1H), 7.65(s, 1H), | | |
| | a]pyridine | 7.52(m, 2H), 7.21(t, J=5Hz, 1H), 7.03(d, J=8Hz, | | |
| | | 1H), 2.68(s, 3H). | | |
| Ex. 63 | 4-Methyl-7-(3-pyridin-2- | ¹H-NMR(300MHz, CDCl₃, δ): 11.00(br s, | 334[M+H]+ | Ex. E, 1, and 2 |
| | yl-1H-pyrazol-4-yl)-3,4- | 1H), 8.60(d, J=4Hz, 1H), 8.05(d, J=1Hz, | | |
| | dihydro-1H- | 1H), 7.72(br s, 1H), 7.66(s, 1H), | | |
| | benzo[e][1,4]diazepine- | 7.59(m, 1H), 7.51(dd, J=2Hz, 8Hz, 1H), | | |
| | 2,5-dione | 7.40(d, J=8Hz, 1H), 7.21(m, 1H), | | |
| | | 6.96(d, J=9Hz, 1H), 3.94(s, 2H), 3.29(s, | | |
| | | 3H). | | |
| Ex. 64 | 2,3-Dimethyl-6-(3- | ¹H-NMR(300MHz, CDCl₃, δ): 11.74(br s, | 318[M+H]+ | Ex. A, 1, and 2 |
| | pyridin-2-yl-1H-pyrazol- | 1H), 8.61(d, J=5Hz, 1H), 8.32(d, J=2Hz, | | |
| | 4-yl)-3H-quinazolin-4- | 1H), 7.74(d, J=2Hz, 1H), 7.71(s, | | |
| | one | 1H), 7.61(d, J=9Hz, 1H), 7.50(td, J=1Hz, | | |
| | | 8Hz, 1H), 7.31(d, J=8Hz, 1H), | | |
| | | 7.18(t, J=7Hz, 1H), 3.62(s, 3H), 2.63(s, | | |
| | | 3H). | | |
| Ex. 65 | 6-[3-(6-Methyl-pyridin-2- | ¹H-NMR(400MHz, CDCl₃, δ): 8.74(s, | 277[M+H]+ | Ex. 5 |
| | yl)-1H-pyrazol-4-yl]- | 1H), 8.38(s, 1H), 7.78(dd, J=1Hz, 9Hz, | | |
| | [1,2,4]triazolo[1,5- | 1H), 7.75(s, 1H), 7.55(m, 2H), 7.20(d, J=8Hz, | | |
| | a]pyridine | 1H), 7.14(d, J=8Hz, 1H), | | |
| | | 2.61(s, 3H). | | |
| Ex. 66 | 1-Methoxy-4-(3-pyridin- | ¹H-NMR(300MHz, CDCl₃, δ): 8.60(d, J=5Hz, | 303[M+H]+ | Ex. I, 1, and 2 |
| | 2-yl-1H-pyrazol-4-yl)- | 1H), 8.34(m, 1H), 8.04(d, J=6Hz, | | |
| | isoquinoline | 1H), 7.68(m, 2H), 7.53(m, 2H), 7.37(t, J=9Hz, | | |
| | | 1H), 7.15(q, J=2Hz, 5Hz, 1H), | | |
| | | 6.90(d, 9Hz, 1H), 4.20(s, 3H). | | |
| Ex. 67 | 2-Methyl-6-(3-pyridin-2- | ¹H-NMR(300MHz, CDCl₃, δ): 9.63(br s, | 277[M+H]+ | Ex. B, 1, and 2 |
| | yl-1H-pyrazol-4-yl)- | 1H), 8.67(d, J=4Hz, 1H), 8.62(s, 1H), | | |
| | [1,2,4]triazolo[1,5- | 7.77(s, 1H), 7.68(m, 2H), 7.53(dd, J=2Hz, | | |
| | a]pyridine | 9Hz, 1H), 7.42(d, J=8Hz, 1H), | | |
| | | 7.30(q, J=1Hz, 6Hz, 1H), 2.64(s, 3H). | | |
| Ex. 68 | 4-(3-Pyridin-2-yl-1H- | ¹H-NMR(300MHz, DMSO-d₆, δ): | 289[M+H]+ | Ex. I, 1, 2, |
| | pyrazol-4-yl)-2H- | 11.40(d, J=6Hz, 1H), 8.48(d, J=6Hz, 1H), | | and 23 |
| | isoquinolin-1-one | 8.25(d, J=7Hz, 1H), 7.80(t, J=8Hz, | | |
| | | 2H), 7.49(m, 3H), 7.33(m, 1H), 7.18(d, | | |
| | | J=8Hz, 1H), 7.11(d, J=8Hz, 1H); | | |
| Ex. 69 | 2-(4-Benzo[1,3]dioxol-5- | ¹H-NMR(300MHz, MeOH-d₄, δ): 8.28(t, | 306.3[M+H]+ | Ex. 19 |
| | yl-1H-pyrazol-3-yl)-6- | 1H), 8.06(d, 1H), 7.96(s, 1H), 7.59(d, | | |
| | propenyl-pyridine | 1H), 7.23-7.11(m, 1H), 6.90-6.82(m, 4H), | | |
| | | 6.02(s, 2H), 1.89(d, 3H) | | |
| Ex. 70 | 2-(4-Benzo[1,3]dioxol-5- | ¹H-NMR(300MHz, MeOH-d₄, δ): 8.29(t, | 308.1[M+H]+ | Ex. 19 |
| | yl-1H-pyrazol-3-yl)-6- | 1H), 8.01(s, 1H), 7.77(d, 1H), 7.66(d, | | |
| | propyl-pyridine | 1H), 6.89-6.81(m, 3H), 6.01(s, 2H), | | |
| | | 3.07(t, 2H), 1.85(m, 2H), 1.07(t, 3H) | | |
| Ex. 71 | 1-[6-(4- | ¹H-NMR(300MHz, MeOH-d₄, δ): 8.25(t, | 310.0[M+H]+ | Ex. 19 |
| | Benzo[1,3]dioxol-5-yl- | 1H), 7.89(s, 1H), 7.85(d, 1H), 7.71(d, | | |
| | 1H-pyrazol-3-yl)-pyridin- | 1H), 6.92-6.89(m, 3H), 6.00(s, 2H), | | |
| | 2-yl]-ethanol | 5.14(q, 1H), 1.58(d, 3H) | | |
| Ex. 72 | 4-Methoxy-6-(3-pyridin- | ¹H-NMR(300MHz, CDCl₃, δ): 11.79(br | 304[M+H]+ | Ex. F, 1, and 2 |
| | 2-yl-1H-pyrazol-4-yl)- | s, 1H), 8.84(d, J=1Hz, 1H), 8.67(dd, J=2Hz, | | |
| | quinazoline | 4Hz, 1H), 8.25(d, J=2Hz, 1H), | | |
| | | 7.96(d, J=9Hz, 1H), 7.86(dt, J=2Hz, | | |
| | | 9Hz, 1H), 7.76(d, J=1Hz, 1H), 7.55(tt, | | |
| | | J=2Hz, 8Hz, 1H), 7.32(d, J=8Hz, | | |
| | | 1H), 7.23(m, 1H), 4.18(s, 3H) | | |
| Ex. 73 | 6-(3-Pyridin-2-yl-1H- | ¹H-NMR(300MHz, CDCl₃, δ): 8.96(dd, J=2Hz, | 273[M+H]+ | Ex. 1 and 2 |
| | pyrazol-4-yl)-quinoline | 4Hz, 1H), 8.67(d, J=5Hz, 1H), | | |
| | | 8.17(dd, J=3Hz, 8Hz, 2H), 7.91(d, J=1Hz, | | |
| | | 1H), 7.78(m, 2H), 7.54(td, J=1Hz, | | |
| | | 7Hz, 1H), 7.46(m, 1H), 7.37(t, J=8Hz, | | |
| | | 1H), 7.25(m, 1H) | | |
| Ex. 74 | 6-(3-Pyridin-2-yl-1H- | ¹H-NMR(300MHz, DMSO-d₆, δ): 8.84(d, | 289[M+H]+ | Ex. G, 1 and 2 |
| | pyrazol-4-yl)-quinazolin- | J=1Hz, 1H), 8.67(dd, J=2Hz, 4Hz, | | |
| | 4-ylamine | 1H), 8.25(d, J=2Hz, 1H), 8.00(br s, | | |
| | | 2H), 7.92(d, J=9Hz, 1H), 7.86(dt, J=2Hz, | | |
| | | 9Hz, 1H), 7.76(d, J=1Hz, 1H), | | |
| | | 7.55(tt, J=2Hz, 8Hz, 1H), 7.32(d, J=8Hz, | | |
| | | 1H), 7.23(m, 1H) | | |
| Ex. 75 | 6-(3-Pyridin-2-yl-1H- | ¹H-NMR(300MHz, DMSO-d₆, δ): | 290[M+H]+ | Ex. 1, 2, and |
| | pyrazol-4-yl)-3H- | 13.38(br s, 1H), 12.19(br s, 1H), 8.51(s, 1H), | | 23 |
| | quinazolin-4-one | 8.09(d, J=2Hz, 1H), 8.05(s, 1H), | | |
| | | 7.79(m, 3H), 7.58(d, J=9Hz, 2H), 7.34(t, J=6Hz, | | |
| | | 1H) | | |
| Ex. 76 | 7-(3-Pyridin-2-yl-1H- | ¹H-NMR(300MHz, CDCl₃, δ): 11.22(br | 290[M+H]+ | Ex. H, 1 and 2 |
| | pyrazol-4-yl)-pyrido[1,2- | s, 1H), 9.22(d, J=1Hz, 1H), 8.61(d, J=4Hz, | | |
| | a]pyrimidin-4-one | 1H), 8.32(d, J=6Hz, 1H), 7.80(m, | | |
| | | 2H), 7.68(td, J=2Hz, 7Hz, 2H), 7.52(d, | | |
| | | J=8Hz, 1H), 7.28(m, 1H), 6.48(d, J=7Hz, | | |
| | | 1H) | | |
| Ex. 77 | 6-[3-(6-Cyclopropyl- | ¹H-NMR(300MHz, MeOH-d₄, δ): 9.02(s, | 334.2[M+H]+ | Ex. 5 |
| | pyridin-2-yl)-1H-pyrazol- | 1H), 8.64(s, 1H), 8.21(s, 1H), 8.16(t, | | |
| | 4-yl]-[1,2,4]triazolo[1,5- | 1H), 7.91(d, 1H), 7.82(dd, 1H), 7.61(d, | | |
| | a]pyridine | 1H), 7.42(d, 1H), 2.50(m, 1H), | | |
| | | 1.46-1.32(m, 2H), 1.17-1.08(m, 2H) | | |
| Ex. 78 | 3-Methyl-6-[3-(6-methyl- | ¹H-NMR(300MHz, MeOH-d₄, δ): 8.45(s, | 318.2[M+H]+ | Ex. 5 |
| | pyridin-2-yl)-1H-pyrazol- | 1H), 8.30(t, 1H), 8.21-8.20(m, 2H), | | |
| | 4-yl]-3H-quinazolin-4- | 7.89(dd, 1H), 7.83(d, 1H), 7.77(d, 1H), | | |
| | one | 7.64(d, 1H), 3.63(s, 3H), 2.88(s, 3H) | | |
| Ex. 79 | 2-(4-Benzo[1,3]dioxol-5- | ¹H-NMR(300MHz, DMSO-d₆, δ): 7.89(s, | 308.2[M+H]+ | Ex. 21 |
| | yl-1H-pyrazol-3-yl)-6- | 1H), 7.87(t, 1H), 7.44(d, 1H), 7.35(d, | | |
| | isopropyl-pyridine | 1H), 7.06(s, 1H), 6.87(s, 2H), 5.99(s, | | |
| | | 2H), 3.07(m, 1H), 1.20(d, 6H) | | |
| Ex. 80 | 6-[3-(5-Fluoro-6-methyl- | ¹H-NMR(300MHz, MeOH-d₄, δ): 9.09(s, | 295.3[M+H]+ | Ex. 5 |
| BIO- | pyridin-2-yl)-1H-pyrazol- | 1H), 8.56(s, 1H), 8.05(s, 1H), 7.86(dd, | | |
| 013075-01 | 4-yl]-[1,2,4]triazolo[1,5- | 1H), 7.80(d, 1H), 7.65(dd, 1H), 7.56(t, | | |
| | a]pyridine | 1H), 2.46(d, 3H) | | |
| Ex. 81 | 6-[3-(6-Trifluoromethyl- | ¹H-NMR(300MHz, MeOH-d₄, δ): 8.97(s, | 331.3[M+H]+ | Ex. 5 |
| BIO- | pyridin-2-yl)-1H-pyrazol- | 1H), 8.50(s, 1H), 8.06(d, 1H), | | |
| 013076-01 | 4-yl]-[1,2,4]triazolo[1,5- | 8.00-7.92(m, 2H), 7.86(d, 1H), 7.69(d, 1H), | | |
| | a]pyridine | 7.60(d, 1H) | | |
| Ex. 82 | 6-[3-(6-Methyl-pyridin-2- | ¹H-NMR(300MHz, MeOH-d₄, δ): 8.89(s, | 344.5[M+H]+ | Ex. 5 |
| BIO- | yl)-1H-pyrazol-4-yl]- | 2H), 8.24(m, 2H), 8.12(d, 1H, J=8.7Hz), | | |
| 013077-01 | quinoxaline | 8.05(m, 1H), 7.88(m, 1H), 7.78(d, | | |
| | | 1H, J=7.8Hz), 7.64(d, 1H, J=7.8Hz), | | |
| | | 2.82(s, 3H) | | |
| Ex. 83 | 6-[3-(6-Cyclopropyl- | ¹H-NMR(300MHz, MeOH-d₄, δ): 8.89(s, | 288.3[M+H]+ | Ex. 5 |
| BIO- | pyridin-2-yl)-1H-pyrazol- | 2H), 8.24(m, 2H), 8.12(d, 1H, J=8.7Hz), | | |
| 013078-01 | 4-yl]-3-methyl-3H- | 8.05(m, 1H), 7.88(m, 1H), 7.78(d, | | |
| | quinazolin-4-one | 1H, J=7.8Hz), 7.64(d, 1H, J=7.8Hz), | | |
| | | 2.82(s, 3H) | | |
| Ex. 84 | 6-(3-Pyridin-2-yl-1H- | ¹H-NMR(300MHz, DMSO-d₆, δ): | 264[M+H]+ | Ex. 1 and 2 |
| BIO- | pyrazol-4-yl)- | 13.38(br s, 1H), 8.40(s, 1H), 8.31(d, J=2Hz, | | |
| 013168-00 | [1,2,4]triazolo[1,5- | 1H), 7.99(d, J=4Hz, 2H), 7.75(m, 3H), | | |
| | b]pyridazine | 7.08(t, J=6Hz, 1H) | | |
| Ex. 85 | 6-[3-(6-Methyl-pyridin-2- | ¹H-NMR(300MHz, MeOH-d₄, δ): 9.13(m, | 287.3[M+H]+ | Ex. 5 |
| BIO- | yl)-1H-pyrazol-4-yl]- | 1H), 8.94(m, 1H), 8.24(m, 4H), 8.09(m, | | |
| 013185-01 | quinoline | 1H), 7.98(m, 1H), 7.75(d, 1H, J=8.1Hz), | | |
| | | 7.60(d, 1H, J=7.8Hz), 2.80(s, 3H) | | |
| Ex. 86 | 6-(4-Benzo[1,3]dioxol-5- | ¹H-NMR(300MHz, MeOH-d₄, δ): 7.76(s, | 298.3[M+H]+ | Ex. 5 |
| BIO- | yl-1H-pyrazol-3-yl)-3- | 1H), 7.61(t, 1H), 7.37(dd, 1H), | | |
| 013203-01 | fluoro-2-methyl-pyridine | 6.88-6.81(m, 3H), 5.97(s, 2H), 2.60(s, 3H) | | |
| Ex. 87 | 7-Methoxy-3-methyl-6- | n/a | 334[M+H]⁺ | Ex. 1 and 2 |
| BIO- | (3-pyridin-2-yl-1H- | | | |
| 013209-00 | pyrazol-4-yl)-3H- | | | |
| | quinazolin-4-one | | | |
| Ex. 88 | (4-Morpholin-4-yl- | ¹H-NMR(300MHz, DMSO-d₆, δ): 8.84(d, | 450[M+H]+ | Ex. 1 and 2 |
| BIO | phenyl)-[6-(3-pyridin-2- | J=1Hz, 1H), 8.67(dd, J=2Hz, 4Hz, | | |
| 013220-00 | yl-1H-pyrazol-4-yl)- | 1H), 8.25(d, J=2Hz, 1H), 8.00(br s, | | |
| | quinazolin-4-yl]-amine | 2H), 7.92(d, J=9Hz, 1H), 7.86(dt, J=2Hz, | | |
| | | 9Hz, 1H), 7.76(d, J=1Hz, 1H), | | |
| | | 7.55(tt, J=2Hz, 8Hz, 1H), 7.32(d, J=8Hz, | | |
| | | 1H), 7.27(m, 2H), 7.23(m, 3H), 3.81(m, | | |
| | | 4H), 2.85(m, 4H) | | |
| Ex. 89 | 4-Isopropoxy-6-(3- | ¹H-NMR(400MHz, DMSO-d₆, δ): | 332[M+H]+ | Ex. 1 and 2 |
| BIO- | pyridin-2-yl-1H-pyrazol- | 13.30(br s, 1H), 8.71(d, J=6Hz, 1H), 8.45(s, | | |
| 013298-00 | 4-yl)-quinazoline | 1H), 8.27(s, 1H), 8.14(m, 1H), 7.93(t, J=6Hz, | | |
| | | 2H), 7.82(m, 2H), 7.33(s, 1H), | | |
| | | 5.50(m, 1H), 1.39(s, 6H) | | |
| Ex. 90 | 6-(3-Pyridin-2-yl-1H- | n/a | 288[M+H]+ | Ex. 1 and 2 |
| BIO- | pyrazol-4-yl)-quinolin-4- | | | |
| 013299-00 | ylamine | | | |
| Ex. 91 | {4-[4-Benzo[1,3]dioxol- | n/a | 511[M+H]+ | Ex. 6 |
| BIO- | 5-yl-3-(6-methyl-pyridin- | | | |
| 013303-00 | 2-yl)-pyrazol-1-yl]- | | | |
| | cyclohexyl}-carbamic | | | |
| | acid benzyl ester | | | |
| Ex. 92 | 4-[4-Benzo[1,3]dioxol-5- | n/a | 377[M+H]+ | Scheme 1 |
| BIO- | yl-3-(6-methyl-pyridin-2- | | | |
| 013307-01 | yl)-pyrazol-1-yl]- | | | |
| | cyclohexylamine | | | |
| Ex. 93 | N-{4-[4- | n/a | 455[M+H]+ | Ex. 13 |
| BIO- | Benzo[1,3]dioxol-5-yl-3- | | | |
| 013314-00 | (6-methyl-pyridin-2-yl)- | | | |
| | pyrazol-1-yl]- | | | |
| | cyclohexyl}- | | | |
| | methanesulfonamide | | | |
| Ex. 94 | 6-[3-(5-Fluoro-6-methyl- | ¹H-NMR(300MHz, MeOH-d₄, δ): 8.84(s, | 306.2[M+H]+ | Ex. 5 |
| BIO- | pyridin-2-yl)-1H-pyrazol- | 1H), 8.83(s, 1H), 8.09(s, 1H), | | |
| 013317-01 | 4-yl]-quinoxaline | 8.04-8.01(m, 2H), 7.84(dt, 1H), 7.63(dt, 1H), | | |
| | | 7.50(dd(br), 1H), 2.25(d, 3H) | | |
| Ex. 95 | 7-(3-Pyridin-2-yl-1H- | ¹H-NMR(300MHz, CDCl₃, δ): 8.69(d, J=5Hz, | 263[M+H]+ | Ex. 1 and 2 |
| BIO- | pyrazol-4-yl)- | 1H), 8.61(d, J=7Hz, 1H), | | |
| 013323-00 | [1,2,4]triazolo[1,5- | 8.39(s, 1H), 7.86(s, 1H), 7.81(s, 1H), 7.67(t, | | |
| | a]pyridine | J=8Hz, 1H), 7.50(d, J=7Hz, 1H), | | |
| | | 7.30(t, J=6Hz, 1H), 7.12(dd, J=2Hz, 7Hz, | | |
| | | 1H) | | |
| Ex. 96 | 5-(3-Pyridin-2-yl-1H- | ¹H-NMR(300MHz, CDCl₃, δ): 8.62(d, J=4Hz, | 280[M+H]+ | Ex. 1 and 2 |
| BIO- | pyrazol-4-yl)- | 1H), 7.85(s, 1H), 7.72(s, 1H), | | |
| 013326-00 | benzo[1,2,5]thiadiazole | 7.64(d, J=7Hz, 1H), 7.50(m, 2H), | | |
| | | 7.31(d, J=7Hz, 1H), 7.20(dd, J=5Hz, 8Hz, | | |
| | | 1H) | | |
| Ex. 97 | 5-(3-Pyridin-2-yl-1H- | ¹H-NMR(300MHz, CDCl₃, δ): 8.67(d, J=4Hz, | 264[M+H]+ | Ex. 1 and 2 |
| BIO- | pyrazol-4-yl)- | 1H), 7.80(s, 1H), 7.74(s, 1H), | | |
| 013337-00 | benzo[1,2,5]oxadiazole | 7.66(d, J=7Hz, 1H), 7.51(m, 2H), | | |
| | | 7.34(d, J=7Hz, 1H), 7.22(dd, J=5Hz, 8Hz, | | |
| | | 1H) | | |
| Ex. 98 | 5-(3-Pyridin-2-yl-1H- | ¹H-NMR(300MHz, CDCl₃, δ): 8.65(d, J=4Hz, | 263[M+H]+ | Ex. 1 and 2 |
| BIO- | pyrazol-4-yl)- | 1H), 8.17(s, 1H), 7.87(s, 1H), | | |
| 013339-00 | benzooxazole | 7.70(s, 1H), 7.65(d, J=8Hz, 1H), | | |
| | | 7.50(m, 2H), 7.31(d, J=8Hz, 1H), 7.18(dd, J=5Hz, | | |
| | | 8Hz, 1H) | | |
| Ex. 99 | 6-[3-(6-Trifluoromethyl- | ¹H-NMR(300MHz, CDCl₃, δ): 9.91(s, | 342.03[M+H]+ | Ex. 5 |
| BIO- | pyridin-2-yl)-1H-pyrazol- | 2H), 8.22(d, 1H), 8.19(d, 1H), 7.89(dd, | | |
| 013366-01 | 4-yl]-quinoxaline | 1H), 7.87(s, 1H), 7.77(t, 1H), 7.65(d, 1H), | | |
| | | 7.62(d, 1H) | | |
| Ex. 100 | 5-[3-(6-Methyl-pyridin-2- | ¹H-NMR(300MHz, DMSO-d₆, δ): 8.25(s, | 293.92[M+H]+ | Ex. 5 |
| BIO- | yl)-1H-pyrazol-4-yl]- | 1H), 8.14(s, 1H), 8.02(dd, 1H), 7.87(dt, | | |
| 013384-01 | benzo[1,2,5]thiadiazole | 1H), 7.74(d, 1H), 7.53(d, 1H), 7.37(d, 1H), | | |
| | | 7.62(d, 1H), 2.50(s, 3H) | | |
| Ex. 101 | 6-(3-Pyridin-2-yl-1H- | ¹H-NMR(300MHz, CDCl₃, δ): 9.05(s, | 279[M+H]+ | Ex. 1 and 2 |
| BIO- | pyrazol-4-yl)- | 1H), 8.70(d, J=5Hz, 1H), 8.19(d, J=9Hz, | | |
| 013387-00 | benzothiazole | 1H), 8.04(d, J=2Hz, 1H), 7.78(s, | | |
| | | 1H), 7.60(m, 2H), 7.35(d, J=8Hz, 1H), | | |
| | | 7.27(t, J=6Hz, 1H) | | |
| Ex. 102 | 3-(3-Methoxy-phenyl)-5- | ¹H-NMR(300MHz, CDCl₃, δ): 8.70(d, J=4Hz, | 369[M+H]+ | Ex. 1 and 2 |
| BIO- | (3-pyridin-2-yl-1H- | 1H), 7.93(s, 1H), 7.78(s, 1H), | | |
| 013392-00 | pyrazol-4-yl)- | 7.66(m, 2H), 7.58(m, 1H), 7.55(m, 1H), | | |
| | benzo[c]isoxazole | 7.47(s, 1H), 7.44(s, 1H), 7.38(d, J=2Hz, | | |
| | | 1H), 7.35(m, 1H), 7.05(dd, J=2Hz, | | |
| | | 8Hz, 1H), 3.90(s, 3H) | | |
| Ex. 103 | 5-[3-(6-Methyl-pyridin-2- | ¹H-NMR(300MHz, MeOH-d₄, δ): 8.30(t, | 352.3[M+H]+ | Ex. 5 |
| BIO- | yl)-1H-pyrazol-4-yl]-3- | 1H), 8.17(s, 1H), 7.96(d, 1H), 7.83(m, | | |
| 013396-01 | phenyl- | 3H), 7.69(d, 1H), 7.60-7.52(m, 4H), | | |
| | benzo[c]isoxazole | 7.35(d, 1H), 2.67(s, 3H) | | |
| Ex. 104 | 3-(4-Methoxy-phenyl)-5- | ¹H-NMR(300MHz, CDCl₃, δ): 8.67(d, J=4Hz, | 369[M+H]+ | Ex. 1 and 2 |
| BIO- | (3-pyridin-2-yl-1H- | 1H), 7.98(d, J=8Hz, 2H), | | |
| 013409-00 | pyrazol-4-yl)- | 7.90(s, 1H), 7.75(s, 1H), 7.62(m, 2H), | | |
| | benzo[c]isoxazole | 7.43(d, J=8Hz, 1H), 7.34(dd, J=2Hz, 9Hz, | | |
| | | 1H), 7.26(m, 1H), 7.07(d, J=8Hz, 2H), | | |
| | | 3.91(s, 3H). | | |
| Ex. 105 | 3-(4-Chloro-phenyl)-5- | ¹H-NMR(300MHz, DMSO-d₆, δ): | 374[M+H]+ | Ex. 1 and 2 |
| BIO- | (3-pyridin-2-yl-1H- | 13.32(br s, 1H), 8.54(s, 1H), 8.25(d, J=8Hz, | | |
| 013414-00 | pyrazol-4-yl)- | 2H), 8.12(s, 1H), 8.04(s, 1H), 7.85(m, | | |
| | benzo[c]isoxazole | 2H), 7.67(d, J=8Hz, 1H), 7.57(dd, J=2Hz, | | |
| | | 9Hz, 1H), 7.39(m, 1H), 7.17(d, J=8Hz, | | |
| | | 2H) | | |
| Ex. 106 | 3-(4-Ethyl-phenyl)-5-(3- | ¹H-NMR(300MHz, CDCl₃, δ): 8.68(m, | 367[M+H]+ | Ex. 1 and 2 |
| BIO- | pyridin-2-yl-1H-pyrazol- | 1H), 7.96(s, 1H), 7.93(s, 3H), 7.76(s, | | |
| 013416-00 | 4-yl)-benzo[c]isoxazole | 1H), 7.63(m, 2H), 7.44(d, J=8Hz, 1H), | | |
| | | 7.38(m, 2H), 7.25(m, 1H), 2.76(q, J=8Hz, | | |
| | | 15Hz, 2H), 1.31(t, J=8Hz, 3H) | | |
| Ex. 107 | 5-(3-Pyridin-2-yl-1H- | ¹H-NMR(300MHz, DMSO-d₆, δ): | 345[M+H]+ | Ex. 1 and 2 |
| BIO- | pyrazol-4-yl)-3- | 13.37(br s, 1H), 8.59(d, J=4Hz, 1H), 8.21(s, | | |
| 013425-00 | thiophen-3-yl- | 1H), 8.00(m, 2H), 7.90(d, J=8Hz, 1H), | | |
| | benzo[c]isoxazole | 7.80(td, J=2Hz, 8Hz, 1H), 7.56(m, | | |
| | | 2H), 7.54(s, 1H), 7.33(m, 1H), 7.28(s, | | |
| | | 1H) | | |
| Ex. 108 | 5-(3-Pyridin-2-yl-1H- | ¹H NMR(300MHz, acetone-d₆, δ): | 306[M+H]+ | Ex. 1 and 2 |
| BIO- | pyrazol-4-yl)-1H- | 8.61(d, J=5Hz, 1H), 8.29(s, 1H), 7.83(s, 1H), | | |
| 013492 | indazole-3-carboxylic | 7.77-7.67(m, 2H), 7.55-7.47(m, 2H), | | |
| | acid | 7.32(m, 1H) | | |
| Ex. 109 | 5-(3-Pyridin-2-yl-1H- | ¹H NMR(300MHz, acetone-d₆, δ): | 319[M+H]+ | Ex. 1 and 2 |
| BIO- | pyrazol-4-yl)-1H- | 8.60(d, J=5Hz, 1H), 8.41(s, 1H), 7.79(s, 1H), | | |
| 013512 | indazole-3-carboxylic | 7.72(t, J=8Hz, 1H), 7.62(d, J=9Hz, 1H), | | |
| | acid methylamide | 7.53-7.42(m, 2H), 7.32(m, 1H), 2.95(s, | | |
| | | 3H) | | |
| Ex. 110 | 5-(3-Pyridin-2-yl-1H- | ¹H NMR(300MHz, acetone-d₆, δ): | 333[M+H]+ | Ex. 1 and 2 |
| BIO- | pyrazol-4-yl)-1H- | 8.57(d, J=5Hz, 1H), 8.22(s, 1H), 7.74(s, 1H), | | |
| 013524 | indazole-3-carboxylic | 7.71-7.59(m, 2H), 7.52-7.41(m, 2H), | | |
| | acid dimethylamide | 7.26(m, 1H), 3.45(s, 3H), 3.10(s, 3H) | | |
| Ex. 111 | 5-(3-Pyridin-2-yl-1H- | n/a | 375[M+H]+ | Ex. 1 and 2 |
| BIO- | pyrazol-4-yl)-1H- | | | |
| 013525 | indazole-3-carboxylic | | | |
| | acid (2,2-dimethyl- | | | |
| | propyl)-amide | | | |
| Ex. 112 | 5-(3-Pyridin-2-yl-1H- | n/a | 381[M+H]+ | Ex. 1 and 2 |
| BIO- | pyrazol-4-yl)-1H- | | | |
| 013526 | indazole-3-carboxylic | | | |
| | acid phenylamide | | | |
| Ex. 113 | Morpholin-4-yl-[5-(3- | ¹H NMR(300MHz, acetone-d₆, δ): | 375[M+H]+ | Ex. 1 and 2 |
| BIO- | pyridin-2-yl-1H-pyrazol- | 8.54(d, J=5Hz, 1H), 7.97(s, 1H), 7.83(s, 1H), | | |
| 013527 | 4-yl)-1H-indazol-3-yl]- | 7.75(t, J=7Hz, 1H), 7.59-7.44(m, 2H), | | |
| | methanone | 7.32(s, 1H), 7.31(m, 1H), 3.65-3.31(m, | | |
| | | 8H) | | |
| Ex. 114 | 5-(3-Pyridin-2-yl-1H- | n/a | 395[M+H]+ | Ex. 1 and 2 |
| BIO- | pyrazol-4-yl)-1H- | | | |
| 013528 | indazole-3-carboxylic | | | |
| | acid benzylamide | | | |
| Ex. 115 | 5-(3-Pyridin-2-yl-1H- | n/a | 373[M+H]+ | Ex. 1 and 2 |
| BIO- | pyrazol-4-yl)-1H- | | | |
| 013529 | indazole-3-carboxylic | | | |
| | acid cyclopentylamide | | | |

The TGFβ or activin inhibitory activity of compounds of formula (I) can be assessed by methods described in the following examples.

### EXAMPLE 116

Cell-Free Assay for Evaluating Inhibition of Autophosphorylation of TGFβ Type I Receptor

The serine-threonine kinase activity of TGFβ type I receptor was measured as the autophosphorylation activity of the cytoplasmic domain of the receptor containing an N-terminal poly histidine, TEV cleavage site-tag, e.g., His-TGFβRI. The His-tagged receptor cytoplasmic kinase domains were purified from infected insect cell cultures using the Gibco-BRL FastBac HTb baculovirus expression system.

To a 96-well Nickel FlashPlate (NEN Life Science, Perkin Elmer) was added 20 μl of 1.25 μCi ³³P-ATP/25 μM ATP in assay buffer (50 mM Hepes, 60 mM NaCl, 1 mM MgCl₂, 2 mM DTT, 5 mM MnCl₂% glycerol, and 0.015% Brij® 35). 10 μl of test compounds of formula (I) prepared in 5% DMSO solution were added to the FlashPlate. The assay was then initated with the addition of 20 ul of assay buffer containing 12.5 ρmol of His-TGFβRI to each well. Plates were incubated for 30 minutes at room temperature and the reactions were then terminated by a single rinse with TBS. Radiation from each well of the plates was measured using TopCount (PerkinElmer Lifesciences, Inc., Boston Mass.). Total binding (no inhibition) was defined as counts measured in the presence of DMSO solution containing with no test compound and non-specific binding was defined as counts measured in the presence of EDTA or no-kinase control.

Alternatively, the reaction performed using the above reagents and incubation conditions but in a microcentrifuge tube was analyzed by separation on a 4-20% SDS-PAGE gel and the incorporation of radiolabel into the 40 kDa His-TGFβRI SDS-PAGE band was quantitated on a Storm Phosphoimager (Molecular Dynamics).

Compounds of formula (I) typically exhibited IC₅₀ values of less than 10 μM; some exhibited IC₅₀ values of less than 1.0 VM; and some even exhibited IC₅₀ values of less than 0.1 μM.

### EXAMPLE 117

Cell-Free Assay for Evaluating Inhibition of Activin Type I Receptor Kinase Activity

Inhibition of the Activin type I receptor (Alk 4) kinase autophosphorylation activity by test compounds of formula (I) can be determined in a similar manner as described above in Example 116 except that a similarly His-tagged form of Alk 4 (His-Alk 4) was used in place of the His-TGFβRI.

### EXAMPLE 118

TGFβ Type I Receptor Ligand Displacement FlashPlate Assay

50 nM of tritiated 4-(3-pyridin-2-yl-1H-pyrazol-4-yl)-quinoline (custom-ordered from PerkinElmer Life Science, Inc., Boston, Mass.) in assay buffer (50 mM Hepes, 60 mM NaCl₂, 1 mM MgCl₂, 5 mM MnCl₂, 2 mM 1,4-dithiothreitol (DTT), 2% Brij® 35; pH 7.5) was premixed with a test compound of formula (I) in 1% DMSO solution in a v-bottom plate. Control wells containing either DMSO without test compound or control compound in DMSO were used. To initiate the assay, His-TGFβ Type I receptor in the same assay buffer (Hepes, NaCl₂, MgCl₂, MnCl₂, DTT, and 30% Brij® added fresh) was added to nickel coated FlashPlate (PE, NEN catalog number: SMP107), while the control wells contained only buffer (i.e., no His-TGFβ Type I receptor). The premixed solution of tritiated 4-(3-pyridin-2-yl-1H-pyrazol-4-yl)-quinoline and test compound of formula (I) was then added to the wells. The wells were aspirated after an hour at room temperature and radioactivity in wells (emitted from the tritiated compound) was measured using TopCount (PerkinElmer Lifesciences, Inc., Boston Mass.).

Compounds of formula (I) typically exhibited Kᵢ values of less than 10 μM; some exhibited IC₅₀ values of less than 1.0 μM; and some even exhibited IC₅₀ values of less than 0.1 μM.

### EXAMPLE 119

Assay for Evaluating Cellular Inhibition of TGFβ Signaling and Cytotoxicity

Biological activity of compounds of formula (I) were determined by measuring their ability to inhibit TGFβ-induced PAI-Luciferase reporter activity in HepG2 cells.

HepG2 cells were stably transfected with the PAI-luciferase reporter grown in DMEM medium containing 10% FBS, penicillin (100 U/ml), streptomycin (100 μg/ml), L-glutamine (2 mM), sodium pyruvate (1 mM), and non essential amino acids (1×). The transfected cells were then plated at a concentration of 2.5×10⁴ cells/well in 96 well plates and starved for 3-6 hours in media with 0.5% FBS at 37° C. in a 5% CO₂ incubator. The cells were then stimulated with ligand either 2.5 ng/ml TGFβ in the starvation media containing 1% DMSO and the presence or absence of test compounds of of formula (I) and incubated as described above for 24 hours. The media was washed out in the following day and the luciferase reporter activity was detected using the LucLite Luciferase Reporter Gene Assay kit (Packard, cat. no. 6016911) as recommended. The plates were read on a Wallac Microbeta plate reader, the reading of which was used to determine the IC₅₀ values of compounds of formula (I) for inhibiting TGFβ-induced PAI-Luciferase reporter activity in HepG2 cells. Compounds of formula (I) typically exhibited IC₅₀ values of less 10 uM.

Cytotoxicity was determined using the same cell culture conditions as described above. Specifically, cell viability was determined after overnight incubation with the CytoLite cell viability kit (Packard, cat. no. 6016901). Compounds of formula (I) typically exhibited LD₂₅ values greater than 10 μM.

### EXAMPLE 120

Assay for Evaluating Cellular Inhibition of TGFβ Signaling

The cellular inhibition of activin signaling activity by test compounds of formula (I) were determined in a similar manner as described above in Example 119 except that 100 ng/ml of activin is added to serum starved cells in place of the 2.5 ng/ml TGFβ.

### EXAMPLE 121

Assay for TGFβ-Induced Collagen Expression

Preparation of Immortalized Collagen Promotor-Green Fluorescent Protein Cells

Fibroblasts were derived from the skin of adult transgenic mice expressing Green Fluorescent Protein (GFP) under the control of the collagen 1A1 promoter (see Krempen, K. et al., Gene Exp. 8: 151-163 (1999)). Cells were immortalised with a temperature sensitive large T antigen that is active at 33° C. Cells are expanded at 33° C. then transferred to 37° C. so that the large T becomes inactive (see Xu, S. et al., Exp. Cell Res. 220: 407-414 (1995)). Over the course of about 4 days and one split, the cells cease proliferating. Cells are then frozen in aliquots sufficient for a single 96 well plate.

Assay of TGFβ-Induced Collagen-GFP Expression

Cells are thawed, plated in complete DMEM (contains nonessential amino acids, 1 mM sodium pyruvate and 2 mM L-glutamine) with 10% fetal calf serum and incubated overnight at 37° C., 5% CO₂. The following day, the cells are trypsinized and transferred into 96 well format with 30,000 cells per well in 50 μl complete DMEM containing 2% fetal calf serum, but without phenol red. The cells are incubated at 37° C. for 3 to 4 hours to allow them to adhere to the plate, solutions containing test compounds of formula (I) are then added to triplicate wells with no TGFβ, as well as triplicate wells with 1 ng/ml TGFβ. DMSO was also added to all of the wells at a final concentration of 0.1%. GFP fluorescence emission at 530 nm following excitation at 485 nm was measured at 48 hours after the addition of solution containing test compounds on a CytoFluor microplate reader (PerSeptive Biosystems). The data are then expressed as the ratio of TGFβ-induced to non-induced for each test sample.

### Other Embodiments

It is to be understood that while the invention has been described in conjunction with the detailed description thereof, the foregoing description is intended to illustrate not limit the scope of the invention, which is defined by the scope of the appended claims. Other aspects, advantages, and modifications are within the scope of the following claims.

1. A compound of formula (I): 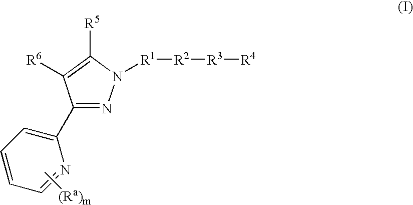
or an N-oxide or a pharmaceutically acceptable salt thereof,
wherein
each R^{a} is independently alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, or heteroaroyl;
R¹ is a bond, alkylene, alkenylene, alkynylene, or —(CH₂)ᵣ₁—O—(CH₂)ᵣ₂—, where each of r1 and r2 is independently 2 or 3;
R² is cycloalkyl, heterocycloalkyl, cycloalkenyl, heterocycloalkenyl, aryl, heteroaryl, or a bond;
R³ is —C(O)—, —C(O)O—, —OC(O)—, —C(O)—N(R^{b})—, —N(R^{b})—C(O)—, —O—C(O)—N(R^{b})—, —N(R^{b})—C(O)—O—, —O—S(O)ₚ—N(R^{b})—, —N(R^{b})—S(O)ₚ—O—, —N(R^{b})—C(O)—N(R^{c})—, —N(R^{b})—S(O)ₚ—N(R^{b})—, —C(O)—N(R^{b})—S(O)ₚ—, —S(O)ₚ—N(R^{b})—C(O)—, —S(O)ₚ—N(R^{b})—, —N(R^{b})—S(O)ₚ—, —N(R^{b})—, —S(O)ₚ—, —O—, —S—, or —(C(R^{b})(R^{c}))_{q}—, or a bond; wherein each of R^{b} and R^{c} is independently hydrogen, hydroxy, alkyl, aryl, aralkyl, heterocycloalkyl, heteroaryl, or heteroaralkyl; p is 1 or 2; and q is 1-4;
R⁴ is hydrogen, alkyl, alkenyl, alkynyl, cycloalkyl, (cycloalkyl)alkyl, heterocycloalkyl, (heterocycloalkyl)alkyl, cycloalkenyl, (cycloalkenyl)alkyl, heterocycloalkenyl, (heterocycloalkenyl)alkyl, aryl, aralkyl, heteroaryl, or heteroaralkyl;
R⁵ is hydrogen, unsubstituted alkyl, halo-substituted alkyl, alkoxy, alkylsulfinyl, amino, alkenyl, alkynyl, cycloalkyl, cycloalkoxy, cycloalkylsulfinyl, heterocycloalkyl, heterocycloalkoxy, heterocycloalkylsulfinyl, aryl, aryloxy, arylsulfinyl, heteroaryl, heteroaryloxy, or heteroarylsulfinyl;
R⁶ is (1) a 5- to 6-membered heterocyclyl containing 1-3 hetero ring atoms selected from the group consisting of —O—, —S—, —N═, and —NR^{d}—, where R^{d} is hydrogen, alkyl, cycloalkyl, cycloalkylalkyl, aryl, aralkyl, heterocycloalkyl, heterocycloalkylalkyl, heteroaryl, or heteroaralkyl; said heterocyclyl being substituted with R^{e} and optionally substituted with one to two R^{f}; where R^{e} is oxo, thioxo, alkoxy, alkylsulfinyl, —NH₂, —NH(unsubstituted alkyl), or —N(unsubstituted alkyl)₂, and R^{f} is alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, aminocarbonyl, alkylcarbonylamino, alkylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, heterocycloalkyl, heterocycloalkyloxy, heterocycloalkylsulfanyl, aryl, aryloxy, arylsulfanyl, aroyl, heteroaryl, heteroaryloxy, heteroarylsulfanyl, or heteroaroyl; or
(2) a fused ring heteroaryl selected from the group consisting of: 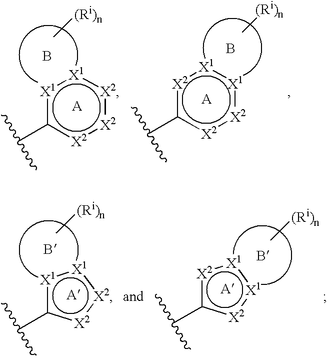
where ring A is an aromatic ring containing 0-4 hetero ring atoms, and ring B is a 5- to 7-membered aromatic or nonaromatic ring containing 0-4 hetero ring atoms; provided that at least one of ring A and ring B contains one or more hetero ring atoms; ring A′ is an aromatic ring containing 0-4 hetero ring atoms, and ring B′ is a 5- to 7-membered saturated or unsaturated ring containing 0-4 hetero ring atoms; provided that at least one of ring A′ and ring B′ contains one or more hetero ring atoms; each hetero ring atom is —O—, —S—, —N═, or —NR^{g} —; each X¹ is independently N or C; each X² is independently —O—, —S—, —N═, —NR^{g}—, or —CHR^{h}—; where R^{g} is hydrogen, alkyl, cycloalkyl, cycloalkylalkyl, aryl, aralkyl, heterocycloalkyl, heterocycloalkylalkyl, heteroaryl, or heteroaralkyl; each of R^{h} and Rⁱ is independently alkyl, alkenyl, alkynyl, alkoxy, acyl, halo, hydroxy, amino, nitro, oxo, thioxo, cyano, guanadino, amidino, carboxy, sulfo, mercapto, alkylsulfanyl, alkylsulfinyl, alkylsulfonyl, aminocarbonyl, alkylcarbonylamino, arylcarbonylamino, heteroarylcarbonylamino, alkylsulfonylamino, arylsulfonylamino, heteroarylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, cycloalkylcarbonyl, heterocycloalkyl, heterocycloalkyloxy, heterocycloalkylsulfanyl, heterocycloalkylcarbonyl, aryl, aryloxy, arylsulfanyl, aroyl, heteroaryl, heteroaryloxy, heteroarylsulfanyl, or heteroaroyl; and n is 0-2; and
m is 0-3; provided that when m≧2, two adjacent R^{a} groups can join together to form a 4- to 8-membered optionally substituted cyclic moiety;
provided that if R⁶ is 2-naphthyridinyl, 4-quinolinyl, imidazo[1,2-a]pyridyl, or benzimidazolyl, then —R¹-R²-R³-R⁴ is not H, unsubstituted alkyl, —CH₂—C(O)—N(H)-alkyl, —CH₂—C(O)—N(alkyl)₂, or benzyl.

2. The compound of claim 1, wherein R⁶ is a 5- to 6-membered heterocyclyl containing 1-3 hetero ring atoms selected from the group consisting of —O—, —S—, —N═, and —NR^{d}— where R is hydrogen or alkyl.

3. The compound of claim 2, wherein R⁶ is a 6-membered heteroaryl containing 1 or 2 hetero ring atoms wherein each hetero ring atom is —N═ or —NR^{d}—.

4. The compound of claim 3, wherein R⁶ is

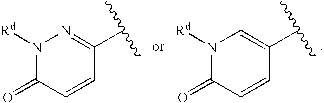

5. The compound of claim 1, wherein R⁶ is

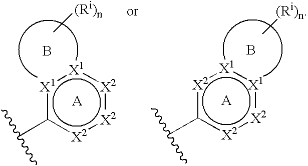

6. The compound of claim 5, wherein ring B is a 5- to 6-membered aromatic or nonaromatic ring.

7. The compound of claim 5, wherein R⁶ contains at least two hetero ring atoms.

8. The compound of claim 5, wherein R⁶ contains at least three hetero ring atoms.

9. The compound of claim 7 or 8, wherein the para-position of ring A is occupied by or substituted with one of said hetero ring atoms or the para-position of ring A is substituted with —OR^{j}, —SR^{j}, —O—CO—R^{j}, —SO₂—R^{j}, —N(R^{j})₂, —NR^{j}—CO—R^{j}, —NR^{j}—SO₂—R^{j}, or —NR^{j}—CO—N(R^{j})₂ where each R^{j} is independently hydrogen, alkyl, cycloalkyl, cycloalkylalkyl, aryl, aralkyl, heterocycloalkyl, heterocycloalkylalkyl, heteroaryl, or heteroaralkyl.

10. The compound of claim 6, wherein R⁶ is 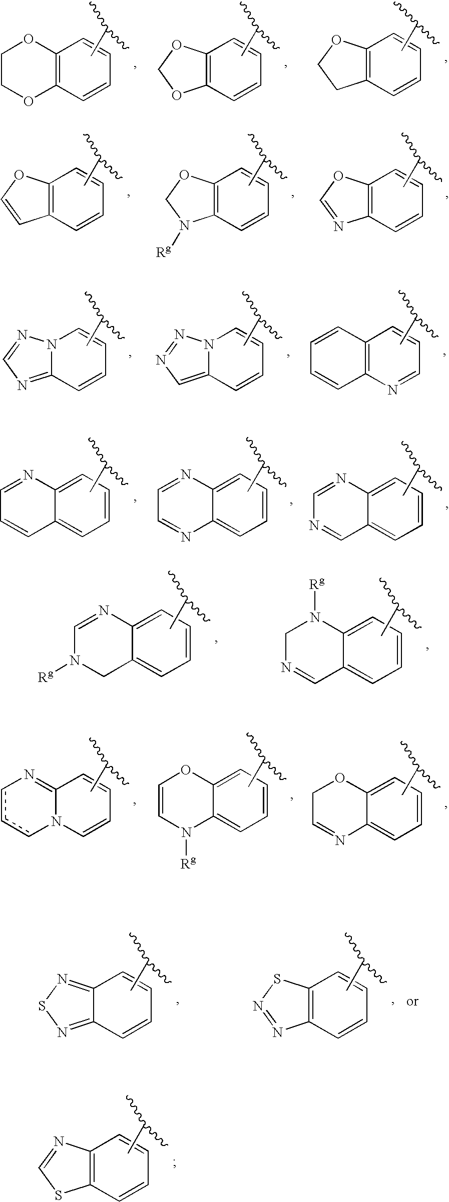
each of which being optionally substituted with alkyl, alkoxy, halo, oxo, thioxo, amino, alkylsulfinyl, cyano, carboxy, aryl, or heteroaryl and R^{g} being hydrogen, alkyl, cycloalkyl, cycloalkylalkyl, aryl, aralkyl, heterocycloalkyl, heterocycloalkylalkyl, heteroaryl, or heteroaralkyl.

11. The compound of claim 10, wherein R⁶ is

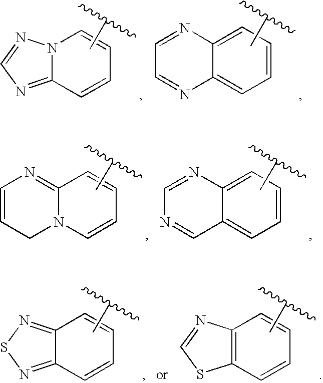

12. The compound of claim 11, wherein R⁶ is

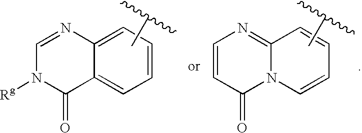

13. The compound of claim 11, wherein R⁶ is

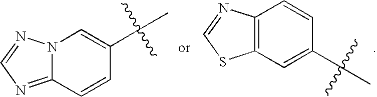

14. The compound of claim 1, wherein R⁶ is

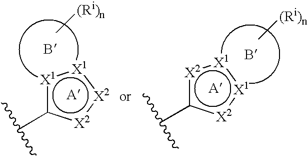

15. The compound of claim 14, wherein ring B′ is a 5- to 6-membered aromatic or nonaromatic ring.

16. The compound of claim 14, wherein R⁶ contains at least two hetero ring atoms.

17. The compound of claim 14, wherein R⁶ contains at least three hetero ring atoms.

18. The compound of claim 15, wherein R⁶ is 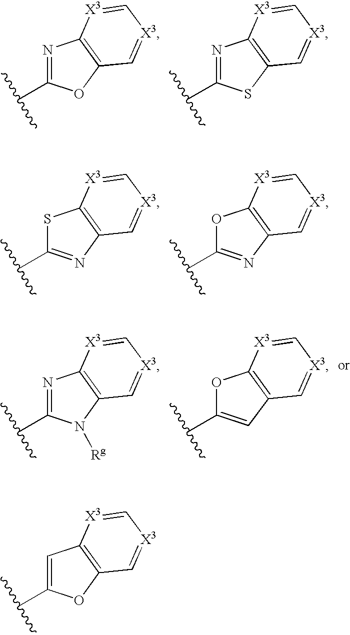
wherein X³ is independently N or C; and each R⁶ is optionally substituted with alkyl, alkoxy, halo, oxo, thioxo, amino, alkylsulfinyl, cyano, carboxy, aryl, or heteroaryl.

19. The compound of claim 1, wherein R¹ is a bond, alkylene, or —(CH₂)₂—O—(CH₂)₂—.

20. The compound of claim 1, wherein R² is cycloalkyl, heterocycloalkyl, aryl, heteroaryl, or a bond.

21. The compound of claim 1, wherein R³ is —N(R^{b})—C(O)—, —N(R^{b})—S(O)ₚ—, —C(O)—, C(O)—O—, —O—C(O)—, —C(O)—N(R^{b})—, —S(O)ₚ—, —O—, —S—, —S(O)ₚ—N(R^{b})—, —N(R^{b})—, —N(R^{b})—C(O)—O—, —N(R^{b})—C(O)—N(R^{b})—, or a bond.

22. The compound of claim 1, wherein R⁴ is hydrogen, alkyl, heterocycloalkyl, aryl, or heteroaryl.

23. The compound of claim 1, wherein R¹ is a bond or alkylene; R² is a bond; R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S(O)ₚ—N(R^{b})—, —N(R^{b})—, or a bond; and R⁴ is hydrogen, alkyl, heterocycloalkyl, aryl, or heteroaryl.

24. The compound of claim 1, wherein R¹ is —(CH₂)₂—O—(CH₂)₂—; R² piperidinyl, piperazinyl, pyrrolidinyl, tetrahydrofuranyl, tetrahydropyranyl, cyclohexyl, cyclopentyl, bicyclo[2.2.1]heptane, bicyclo[2.2.2]octane, bicyclo[3.2. I]octane, 2-oxa-bicyclo[2.2.2]octane, 2-aza-bicyclo[2.2.2]octane, 3-aza-bicyclo[3.2.1]octane, cubanyl, or 1-aza-bicyclo[2.2.2]octane; R³ is a bond; and R⁴ is hydrogen, alkyl, heterocycloalkyl, aryl, or heteroaryl.

25. The compound of claim 1, wherein R¹ is a bond; R² is piperidinyl, piperazinyl, pyrrolidinyl, tetrahydrofuranyl, tetrahydropyranyl, cyclohexyl, cyclopentyl, bicyclo[2.2.1]heptane, bicyclo[2.2.2]octane, bicyclo[3.2.1]octane, 2-oxa-bicyclo[2.2.2]octane, 2-aza-bicyclo[2.2.2]octane, 3-aza-bicyclo[3.2.1]octane, cubanyl, or 1-aza-bicyclo[2.2.2]octane; R³ is —N(R^{b})—C(O)—, —N(R^{b})—S(O)ₚ—, —C(O)—, —C(O)—O—, —O—C(O)—, —C(O)—N(R^{b}), —S(O)ₚ—, —O—, —S—, —S(O)ₚ—N(R^{b})—, —N(R^{b})—, or a bond; and R⁴ is hydrogen, alkyl, heterocycloalkyl, aryl, or heteroaryl.

26. The compound of claim 1, wherein each of R¹, R², and R³ is a bond; and R⁴ is hydrogen or alkyl substituted with cyano.

27. The compound of claim 1, wherein R⁵ is hydrogen, unsubstituted alkyl, or halo-substituted alkyl.

28. The compound of claim 1, wherein m is 0, 1, or 2.

29. The compound of claim 1, wherein R^{a} is substituted at the 6-position.

30. The compound of claim 1, wherein each R^{a} is independently alkyl, alkoxy, alkylsulfinyl, halo, amino, aminocarbonyl, alkoxycarbonyl, cycloalkyl, or heterocycloalkyl.

31. The compound of claim 1, wherein R⁶ is 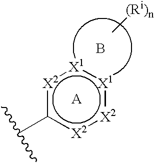
in which ring B is a 5- to 6-membered aromatic or nonaromatic ring; R⁵ is hydrogen, unsubstituted alkyl, or halo-substituted alkyl; R⁴ is hydrogen, alkyl, heterocycloalkyl, aryl, or heteroaryl; R³ is —N(R^{b})—C(O)—, —N(R^{b})—S(O)ₚ—, —C(O)—, —C(O)—O—, —O—C(O)—, —C(O)—N(R^{b})—, —S(O)ₚ—, —O—, —S—, —S(O)ₚ—N(R^{b})—, —N(R^{b})—, or a bond; R² is a bond; R¹ is a bond or alkylene; and R^{a} is alkyl, alkoxy, alkylsulfinyl, halo, amino, aminocarbonyl, or alkoxycarbonyl; provided that if m is not 0, at least one R^{a} is substituted at the 6-position.

32. The compound of claim 31, wherein the para-position of ring A is occupied by or substituted with a hetero ring atom or the para-position of ring A is substituted with —OR^{j}, —SR^{j}, —O—CO—R^{j}, —O—SO₂—R^{j}, —N(R^{j})₂, —NR^{j}—CO—R^{j}, —NR^{j}—SO₂—R^{j}, or —NR^{j}—CO—N(R^{j})₂ where each R^{j} is independently hydrogen, alkyl, cycloalkyl, cycloalkylalkyl, aryl, aralkyl, heterocycloalkyl, heterocycloalkylalkyl, heteroaryl, or heteroaralkyl.

33. The compound of claim 31, wherein R⁶ is 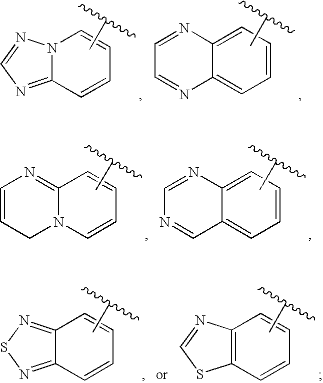
each of which being optionally substituted with alkyl, alkoxy, halo, hydroxy, oxo, amino, alkylsulfinyl, cyano, carboxy, aryl, or heteroaryl.

34. The compound of claim 33, wherein R⁶ is 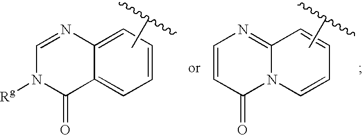
each of which being optionally substituted with alkyl, alkoxy, halo, hydroxy, oxo, amino, alkylsulfinyl, cyano, carboxy, aryl, or heteroaryl.

35. The compound of claim 31, wherein R⁴ is hydrogen or alkyl; R³ is —N(R^{b})—C(O)—, —N(R^{b})—S(O)ₚ—, —C(O)—N(R^{b})—, —S(O)ₚ—N(R^{b})—, —N(R^{b})—, or a bond; R² is cycloalkyl or a bond; R¹ is a bond, alkylene, or —(CH₂)₂—O—(CH₂)₂—.

36. The compound of claim 35, wherein R⁴-R³-R²-R¹- is hydrogen.

37. The compound of claim 34, wherein R⁵ is hydrogen, unsubstituted methyl, or trifluoromethyl.

38. The compound of claim 37, wherein R⁵ is hydrogen.

39. The compound of claim 1, said compound being selected from the group consisting of: 3-(3-pyridin-2-yl-4-quinolin-4-yl-pyrazol-1-yl)-propylamine, N-[3-(3-pyridin-2-yl-4-quinolin-4-yl-pyrazol-1-yl)-propyl]-acetamide, N-[3-(3-pyridin-2-yl-4-quinolin-4-yl-pyrazol-1-yl)-propyl]-methanesulfonamide, dimethyl-[3-(3-pyridin-2-yl-4-quinolin-4-yl-pyrazol-1-yl)-propyl]-amine, 4-{3-pyridin-2-yl-1-[2-(1H-tetrazol-5-yl)-ethyl]-1H-pyrazol-4-yl}-quinoline, 4-[3-pyridin-2-yl-1-(3-pyrrolidin-1-yl-propyl)-1H-pyrazol-4-yl]-quinoline, 5-(3-pyridin-2-yl-1H-pyrazol-4-yl)-pyridin-2-ylamine, 2,4-dimethoxy-5-(3-pyridin-2-yl-1H-pyrazol-4-yl)-pyrimidine, 3-(3-pyridin-2-yl-4-quinolin-4-yl-pyrazol-1-yl)-propionic acid, 5-(3-pyridin-2-yl-1H-pyrazol-4-yl)-1H-indole, 2-[4-(2,3-dihydro-benzo[1,4]dioxin-6-yl)-1H-pyrazol-3-yl]-pyridine, N-hydroxy-3-(3-pyridin-2-yl-4-quinolin-4-yl-pyrazol-1-yl)-propionamide, 2-(3-pyridin-2-yl-4-quinolin-4-yl-pyrazol-1-yl)-ethylamine, N-[2-(3-pyridin-2-yl-4-quinolin-4-yl-pyrazol-1-yl)-ethyl]-methanesulfonamide, 2-methyl-4-methylsulfanyl-6-(3-pyridin-2-yl-1H-pyrazol-4-yl)-pyrimidine, 2-(4-benzo[1,3]dioxol-5-yl-1H-pyrazol-3-yl)-pyridine, 2-[4-(2,3-dihydro-benzofuran-5-yl)-1H-pyrazol-3-yl]-pyridine, 5-(3-pyridin-2-yl-1H-pyrazol-4-yl)-benzo[d]isoxazole, 3-[4-benzo[1,3]dioxol-5-yl-3-(6-methyl-pyridin-2-yl)-pyrazol-1-yl]-propionitrile, N-{3-[4-benzo[1,3]dioxol-5-yl-3-(6-methyl-pyridin-2-yl)-pyrazol-1-yl]-propyl}-methanesulfonamide, 2-[4-(2,3-dihydro-benzo[1,4]dioxin-6-yl)-1H-pyrazol-3-yl]-6-methyl-pyridine, [4-benzo[1,3]dioxol-5-yl-3-(6-methyl-pyridin-2-yl)-pyrazol-1-yl]-acetonitrile, N-{2-[4-benzo[1,3]dioxol-5-yl-3-(6-methyl-pyridin-2-yl)-pyrazol-1-yl]-ethyl}-methanesulfonamide, 4-[3-(6-methyl-pyridin-2-yl)-1H-pyrazol-4-yl]-2-methylsulfanyl-pyrimidine, 4-(3-pyridin-2-yl-1H-pyrazol-4-yl)-2H-phthalazin-1-one, 1-[5-(3-pyridin-2-yl-1H-pyrazol-4-yl)-2,3-dihydro-indol-1-yl]-ethanone, 6-(3-pyridin-2-yl-1H-pyrazol-4-yl)-[1,2,4]triazolo[1,5-a]pyridine, 3-methyl-6-(3-pyridin-2-yl-1H-pyrazol-4-yl)-3H-quinazolin-4-one, 6-(3-pyridin-2-yl-1H-pyrazol-4-yl)-4H-benzo[1,4]oxazin-3-one, 6-(3-pyridin-2-yl-1H-pyrazol-4-yl)-quinoxaline, 3-(4-nitro-benzyl)-6-(3-pyridin-2-yl-1H-pyrazol-4-yl)-3H-quinazolin-4-one, 5-methyl-6-(3-pyridin-2-yl-1H-pyrazol-4-yl)-[1,2,4]triazolo[1,5-a]pyridine, 4-methyl-7-(3-pyridin-2-yl-1H-pyrazol-4-yl)-3,4-dihydro-1H-benzo[e][1,4]diazepine-2,5-dione, 2,3-dimethyl-6-(3-pyridin-2-yl-1H-pyrazol-4-yl)-3H-quinazolin-4-one, 6-[3-(6-methyl-pyridin-2-yl)-1H-pyrazol-4-yl]-[1,2,4]triazolo[1,5-a]pyridine, 1-methoxy-4-(3-pyridin-2-yl-1H-pyrazol-4-yl)-isoquinoline, 2-methyl-6-(3-pyridin-2-yl-1H-pyrazol-4-yl)-[1,2,4]triazolo[1,5-a]pyridine, 4-(3-pyridin-2-yl-1H-pyrazol-4-yl)-2H-isoquinolin-1-one, 2-(4-benzo[1,3]dioxol-5-yl-1H-pyrazol-3-yl)-6-trifluoromethyl-pyridine, 2-(4-benzo[1,3]dioxol-5-yl-1H-pyrazol-3-yl)-6-vinyl-pyridine, 2-(4-benzo[1,3]dioxol-5-yl-1H-pyrazol-3-yl)-6-propenyl-pyridine, 2-(4-benzo[1,3]dioxol-5-yl-1H-pyrazol-3-yl)-6-ethyl-pyridine, 2-(4-benzo[1,3]dioxol-5-yl-1H-pyrazol-3-yl)-6-propyl-pyridine, 2-(4-benzo[1,3]dioxol-5-yl-1H-pyrazol-3-yl)-6-cyclopropyl-pyridine, 1-[6-(4-benzo[1,3]dioxol-5-yl-1H-pyrazol-3-yl)-pyridin-2-yl]-ethanol, 4-methoxy-6-(3-pyridin-2-yl-1H-pyrazol-4-yl)-quinazoline, 6-(3-pyridin-2-yl-1H-pyrazol-4-yl)-quinoline, 6-(3-pyridin-2-yl-1H-pyrazol-4-yl)-quinazolin-4-ylamine, 6-(3-pyridin-2-yl-1H-pyrazol-4-yl)-3H-quinazolin-4-one, 7-(3-pyridin-2-yl-1H-pyrazol-4-yl)-pyrido[1,2-a]pyrimidin-4-one, 6-[3-(6-cyclopropyl-pyridin-2-yl)-1H-pyrazol-4-yl]-[1,2,4]triazolo[1,5-a]pyridine, 3-methyl-6-[3-(6-methyl-pyridin-2-yl)-1H-pyrazol-4-yl]-3H-quinazolin-4-one, 4-(2-{2-[4-benzo[1,3]dioxol-5-yl-3-(6-methyl-pyridin-2-yl)-pyrazol-1-yl]-ethoxy}-ethoxy)-bicyclo[2.2.2]octane-1-carboxylic acid, 4-(2-{2-[4-benzo[1,3]dioxol-5-yl-3-(6-methyl-pyridin-2-yl)-pyrazol-1-yl]-ethoxy}-ethoxy)-bicyclo[2.2.2]octane-1-carboxylic acid methyl ester, 4-[4-benzo[1,3]dioxol-5-yl-3-(6-methyl-pyridin-2-yl)-pyrazol-1-yl]-bicyclo[2.2.2]octane-1-carboxylic acid methyl ester, 2-(4-benzo[1,3]dioxol-5-yl-1H-pyrazol-3-yl)-6-isopropyl-pyridine, 2-(4-benzo[1,3]dioxol-5-yl-5-trifluoromethyl-1H-pyrazol-3-yl)-6-bromo-pyridine, 6-[3-(5-fluoro-6-methyl-pyridin-2-yl)-1H-pyrazol-4-yl]-[1,2,4]triazolo[1,5-a]pyridine, 6-[3-(6-trifluoromethyl-pyridin-2-yl)-1H-pyrazol-4-yl]-[1,2,4]triazolo[1,5-a]pyridine, 6-[3-(6-methyl-pyridin-2-yl)-1H-pyrazol-4-yl]-quinoxaline, 6-[3-(6-cyclopropyl-pyridin-2-yl)-1H-pyrazol-4-yl]-3-methyl-3H-quinazolin-4-one, 6-(3-pyridin-2-yl-1H-pyrazol-4-yl)-[1,2,4]triazolo[1,5-b]pyridazine, 6-[3-(6-methyl-pyridin-2-yl)-1H-pyrazol-4-yl]-quinoline, 6-(4-benzo[1,3]dioxol-5-yl-1H-pyrazol-3-yl)-3-fluoro-2-methyl-pyridine, 7-methoxy-3-methyl-6-(3-pyridin-2-yl-1H-pyrazol-4-yl)-3H-quinazolin-4-one, (4-morpholin-4-yl-phenyl)-[6-(3-pyridin-2-yl-1H-pyrazol-4-yl)-quinazolin-4-yl]-amine, 4-isopropoxy-6-(3-pyridin-2-yl-1H-pyrazol-4-yl)-quinazoline, 6-(3-Pyridin-2-yl-1H-pyrazol-4-yl)-quinolin-4-ylamine, {4-[4-benzo[1,3]dioxol-5-yl-3-(6-methyl-pyridin-2-yl)-pyrazol-1-yl]-cyclohexyl}-carbamic acid benzyl ester, 4-[4-benzo[1,3]dioxol-5-yl-3-(6-methyl-pyridin-2-yl)-pyrazol-1-yl]-cyclohexylamine, N-{4-[4-benzo[1,3]dioxol-5-yl-3-(6-methyl-pyridin-2-yl)-pyrazol-1-yl]-cyclohexyl}-methanesulfonamide, 6-[3-(5-fluoro-6-methyl-pyridin-2-yl)-1H-pyrazol-4-yl]-quinoxaline, 7-(3-pyridin-2-yl-1H-pyrazol-4-yl)-[1,2,4]triazolo[1,5-a]pyridine, 1-tert-butyl-3-[6-(3-pyridin-2-yl-1H-pyrazol-4-yl)-quinazolin-4-yl]-urea, 5-(3-pyridin-2-yl-1H-pyrazol-4-yl)-benzo[1,2,5]thiadiazole, 5-(3-pyridin-2-yl-1H-pyrazol-4-yl)-benzo[1,2,5]oxadiazole, 5-(3-Pyridin-2-yl-1H-pyrazol-4-yl)-benzooxazole, 4-morpholin-4-yl-6-(3-pyridin-2-yl-1H-pyrazol-4-yl)-quinazoline, 6-[3-(6-trifluoromethyl-pyridin-2-yl)-1H-pyrazol-4-yl]-quinoxaline, 4-(4-methoxy-phenyl)-6-(3-pyridin-2-yl-1H-pyrazol-4-yl)-quinazoline, 5-[3-(6-methyl-pyridin-2-yl)-1H-pyrazol-4-yl]-benzo[1,2,5]thiadiazole, 6-(3-pyridin-2-yl-1H-pyrazol-4-yl)-benzothiazole, 3-(3-methoxy-phenyl)-5-(3-pyridin-2-yl-1H-pyrazol-4-yl)-benzo[c]isoxazole, 5-methyl-thiophene-2-carboxylic acid [6-(3-pyridin-2-yl-1H-pyrazol-4-yl)-quinazolin-4-yl]-amide, 5-[3-(6-methyl-pyridin-2-yl)-1H-pyrazol-4-yl]-3-phenyl-benzo[c]isoxazole, 3-(4-methoxy-phenyl)-5-(3-pyridin-2-yl-1H-pyrazol-4-yl)-benzo[c]isoxazole, 3-(4-chloro-phenyl)-5-(3-pyridin-2-yl-1H-pyrazol-4-yl)-benzo[c]isoxazole, 3-(4-ethyl-phenyl)-5-(3-pyridin-2-yl-1H-pyrazol-4-yl)-benzo[c]isoxazole, (4-methoxy-phenyl)-[6-(3-pyridin-2-yl-1H-pyrazol-4-yl)-quinazolin-4-yl]-methanone, 5-(3-pyridin-2-yl-1H-pyrazol-4-yl)-3-thiophen-3-yl-benzo[c]isoxazole, 5-(3-pyridin-2-yl-1H-pyrazol-4-yl)-1H-indazole-3-carboxylic acid, 5-(3-Pyridin-2-yl-1H-pyrazol-4-yl)-1H-indazole-3-carboxylic acid methylamide, 5-(3-pyridin-2-yl-1H-pyrazol-4-yl)-1H-indazole-3-carboxylic acid dimethylamide, 5-(3-pyridin-2-yl-1H-pyrazol-4-yl)-1H-indazole-3-carboxylic acid (2,2-dimethyl-propyl)-amide, 5-(3-pyridin-2-yl-1H-pyrazol-4-yl)-1H-indazole-3-carboxylic acid phenylamide, morpholin-4-yl-[5-(3-pyridin-2-yl-1H-pyrazol-4-yl)-1H-indazol-3-yl]-methanone, 5-(3-pyridin-2-yl-1H-pyrazol-4-yl)-1H-indazole-3-carboxylic acid benzylamide, and 5-(3-pyridin-2-yl-1H-pyrazol-4-yl)-1H-indazole-3-carboxylic acid cyclopentylamide.

40. The compound of claim 1, said compound being selected from the group consisting of: 4-(3-pyridin-2-yl-1H-pyrazol-4-yl)-2H-isoquinolin-1-one, 4-methoxy-6-(3-pyridin-2-yl-1H-pyrazol-4-yl)-quinazoline, 6-(3-pyridin-2-yl-1H-pyrazol-4-yl)-quinoline, 7-(3-pyridin-2-yl-1H-pyrazol-4-yl)-pyrido[1,2-a]pyrimidin-4-one, 6-[3-(6-methyl-pyridin-2-yl)-1H-pyrazol-4-yl]-[1,2,4]triazolo[1,5-a]pyridine, 6-(3-pyridin-2-yl-1H-pyrazol-4-yl)-quinazolin-4-ylamine, 6-(3-pyridin-2-yl-1H-pyrazol-4-yl)-3H-quinazolin-4-one, 6-[3-(6-cyclopropyl-pyridin-2-yl)-1H-pyrazol-4-yl]-[1,2,4]triazolo[1,5-a]pyridine, 3-methyl-6-[3-(6-methyl-pyridin-2-yl)-1H-pyrazol-4-yl]-3H-quinazolin-4-one, 3-methyl-6-(3-pyridin-2-yl-1H-pyrazol-4-yl)-3H-quinazolin-4-one, 2-[4-(2,3-dihydro-benzo[1,4]dioxin-6-yl)-1H-pyrazol-3-yl]-6-methyl-pyridine, 2-(4-benzo[1,3]dioxol-5-yl-1H-pyrazol-3-yl)-6-ethyl-pyridine, 4-(2-{2-[4-benzo[1,3]dioxol-5-yl-3-(6-methyl-pyridin-2-yl)-pyrazol-1-yl]-ethoxy}-ethoxy)-bicyclo[2.2.2]octane-1-carboxylic acid, 2-(4-benzo[1,3]dioxol-5-yl-1H-pyrazol-3-yl)-6-vinyl-pyridine, 4-(2-{2-[4-benzo[1,3]dioxol-5-yl-3-(6-methyl-pyridin-2-yl)-pyrazol-1-yl]-ethoxy}-ethoxy)-bicyclo[2.2.2]octane-1-carboxylic acid methyl ester, 3-[4-benzo[1,3]dioxol-5-yl-3-(6-methyl-pyridin-2-yl)-pyrazol-1-yl]-propionitrile, 2-(4-benzo[1,3]dioxol-5-yl-1H-pyrazol-3-yl)-6-cyclopropyl-pyridine, 2-(4-benzo[1,3]dioxol-5-yl-1H-pyrazol-3-yl)-6-propyl-pyridine, N-[2-(3-pyridin-2-yl-4-quinolin-4-yl-pyrazol-1-yl)-ethyl]-methanesulfonamide, N-{3-[4-benzo[1,3]dioxol-5-yl-3-(6-methyl-pyridin-2-yl)-pyrazol-1-yl]-propyl}-methanesulfonamide, 3-(3-pyridin-2-yl-4-quinolin-4-yl-pyrazol-1-yl)-propionic acid, [4-benzo[1,3]dioxol-5-yl-3-(6-methyl-pyridin-2-yl)-pyrazol-1-yl]-acetonitrile, 6-(3-pyridin-2-yl-1H-pyrazol-4-yl)-4H-benzo[1,4]oxazin-3-one, 4-[3-(6-methyl-pyridin-2-yl)-1H-pyrazol-4-yl]-2-methylsulfanyl-pyrimidine, 5-(3-pyridin-2-yl-1H-pyrazol-4-yl)-benzo[d]isoxazole, N-{2-[4-benzo[1,3]dioxol-5-yl-3-(6-methyl-pyridin-2-yl)-pyrazol-1-yl]-ethyl}-methanesulfonamide, 2-(4-benzo[1,3]dioxol-5-yl-1H-pyrazol-3-yl)-6-trifluoromethyl-pyridine, N-[3-(3-pyridin-2-yl-4-quinolin-4-yl-pyrazol-1-yl)-propyl]-methanesulfonamide, 4-{3-pyridin-2-yl-1-[2-(1H-tetrazol-5-yl)-ethyl]-1H-pyrazol-4-yl}-quinoline, 4-[4-benzo[1,3]dioxol-5-yl-3-(6-methyl-pyridin-2-yl)-pyrazol-1-yl]-bicyclo[2.2.2]octane-1-carboxylic acid methyl ester, 4-(3-pyridin-2-yl-1H-pyrazol-4-yl)-2H-phthalazin-1-one, 3-(4-nitro-benzyl)-6-(3-pyridin-2-yl-1H-pyrazol-4-yl)-3H-quinazolin-4-one, 2-(4-benzo[1,3]dioxol-5-yl-1H-pyrazol-3-yl)-6-propenyl-pyridine, 2-(4-benzo[1,3]dioxol-5-yl-1H-pyrazol-3-yl)-6-isopropyl-pyridine, 1-[6-(4-benzo[1,3]dioxol-5-yl-1H-pyrazol-3-yl)-pyridin-2-yl]-ethanol, 6-(3-pyridin-2-yl-1H-pyrazol-4-yl)-[1,2,4]triazolo[1,5-a]pyridine, 6-(3-pyridin-2-yl-1H-pyrazol-4-yl)-quinoxaline, 5-methyl-6-(3-pyridin-2-yl-1H-pyrazol-4-yl)-[1,2,4]triazolo[1,5-a]pyridine, 2-[4-(2,3-dihydro-benzo[1,4]dioxin-6-yl)-1H-pyrazol-3-yl]-pyridine, 2-(4-benzo[1,3]dioxol-5-yl-1H-pyrazol-3-yl)-pyridine, 2-[4-(2,3-dihydro-benzofuran-5-yl)-1H-pyrazol-3-yl]-pyridine, 2-(4-benzo[1,3]dioxol-5-yl-5-trifluoromethyl-1H-pyrazol-3-yl)-6-bromo-pyridine, 6-[3-(5-Fluoro-6-methyl-pyridin-2-yl)-1H-pyrazol-4-yl]-[1,2,4]triazolo[1,5-a]pyridine, 6-[3-(6-trifluoromethyl-pyridin-2-yl)-1H-pyrazol-4-yl]-[1,2,4]triazolo[1,5-a]pyridine, 6-[3-(6-methyl-pyridin-2-yl)-1H-pyrazol-4-yl]-quinoxaline, 6-[3-(6-cyclopropyl-pyridin-2-yl)-1H-pyrazol-4-yl]-3-methyl-3H-quinazolin-4-one, 6-(3-pyridin-2-yl-1H-pyrazol-4-yl)-[1,2,4]triazolo[1,5-b]pyridazine, 6-[3-(6-methyl-pyridin-2-yl)-1H-pyrazol-4-yl]-quinoline, 6-(4-benzo[1,3]dioxol-5-yl-1H-pyrazol-3-yl)-3-fluoro-2-methyl-pyridine, (4-morpholin-4-yl-phenyl)-[6-(3-pyridin-2-yl-1H-pyrazol-4-yl)-quinazolin-4-yl]-amine, 4-isopropoxy-6-(3-pyridin-2-yl-1H-pyrazol-4-yl)-quinazoline, 6-(3-pyridin-2-yl-1H-pyrazol-4-yl)-quinolin-4-ylamine, 6-[3-(5-fluoro-6-methyl-pyridin-2-yl)-1H-pyrazol-4-yl]-quinoxaline, 7-(3-pyridin-2-yl-1H-pyrazol-4-yl)-[1,2,4]triazolo[1,5-a]pyridine, 1-tert-butyl-3-[6-(3-pyridin-2-yl-1H-pyrazol-4-yl)-quinazolin-4-yl]-urea, 5-(3-pyridin-2-yl-1H-pyrazol-4-yl)-benzo[1,2,5]thiadiazole, 5-(3-pyridin-2-yl-1H-pyrazol-4-yl)-benzo[1,2,5]oxadiazole, 5-(3-pyridin-2-yl-1H-pyrazol-4-yl)-benzooxazole, 4-morpholin-4-yl-6-(3-pyridin-2-yl-1H-pyrazol-4-yl)-quinazoline, 6-[3-(6-trifluoromethyl-pyridin-2-yl)-1H-pyrazol-4-yl]-quinoxaline, 4-(4-methoxy-phenyl)-6-(3-pyridin-2-yl-1H-pyrazol-4-yl)-quinazoline, 5-[3-(6-methyl-pyridin-2-yl)-1H-pyrazol-4-yl]-benzo[1,2,5]thiadiazole, 6-(3-pyridin-2-yl-1H-pyrazol-4-yl)-benzothiazole, 5-methyl-thiophene-2-carboxylic acid [6-(3-pyridin-2-yl-1H-pyrazol-4-yl)-quinazolin-4-yl]-amide, 5-[3-(6-methyl-pyridin-2-yl)-1H-pyrazol-4-yl]-3-phenyl-benzo[c]isoxazolem3-(4-ethyl-phenyl)-5-(3-pyridin-2-yl-1H-pyrazol-4-yl)-benzo[c]isoxazole, 5-(3-pyridin-2-yl-1H-pyrazol-4-yl)-3-thiophen-3-yl-benzo[c]isoxazole, and 5-(3-pyridin-2-yl-1H-pyrazol-4-yl)-1H-indazole-3-carboxylic acid methylamide.

41. A pharmaceutical composition comprising a compound of claim 1 and a pharmaceutically acceptable carrier.

42. A pharmaceutical composition comprising a compound of claim 39 and a pharmaceutically acceptable carrier.

43. A pharmaceutical composition comprising a compound of claim 40 and a pharmaceutically acceptable carrier.

44. A method of inhibiting the TGFβ signaling pathway in a subject, the method comprising administering to said subject with an effective amount of a compound of claim 1.

45. A method of inhibiting the TGFβ signaling pathway in a subject, the method comprising administering to said subject with an effective amount of a compound of claim 39.

46. A method of inhibiting the TGFβ signaling pathway in a subject, the method comprising administering to said subject with an effective amount of a compound of claim 40.

47. A method of inhibiting the TGFβ type I receptor in a cell, the method comprising the step of contacting said cell with an effective amount of a compound of claim 1.

48. A method of inhibiting the TGFβ type I receptor in a cell, the method comprising the step of contacting said cell with an effective amount of a compound of claim 39.

49. A method of inhibiting the TGFβ type I receptor in a cell, the method comprising the step of contacting said cell with an effective amount of a compound of claim 40.

50. A method of reducing the accumulation of excess extracellular matrix induced by TGFβ in a subject, the method comprising administering to said subject an effective amount of a compound of claim 1.

51. A method of reducing the accumulation of excess extracellular matrix induced by TGFβ in a subject, the method comprising administering to said subject an effective amount of a compound of claim 39.

52. A method of reducing the accumulation of excess extracellular matrix induced by TGFβ in a subject, the method comprising administering to said subject an effective amount of a compound of claim 40.

53. A method of treating or preventing fibrotic condition in a subject, the method comprising administering to said subject an effective amount of a compound of claim 1.

54. A method of treating or preventing fibrotic condition in a subject, the method comprising administering to said subject an effective amount of a compound of claim 39.

55. A method of treating or preventing fibrotic condition in a subject, the method comprising administering to said subject an effective amount of a compound of claim 40.

56. The method of claim 53, 54, or 55, wherein the fibrotic condition is selected from the group consisting of scleroderma, lupus nephritis, connective tissue disease, wound healing, surgical scarring, spinal cord injury, CNS scarring, acute lung injury, idiopathic pulmonary fibrosis, chronic obstructive pulmonary disease, adult respiratory distress syndrome, acute lung injury, drug-induced lung injury, glomerulonephritis, diabetic nephropathy, hypertension-induced nephropathy, hepatic or biliary fibrosis, liver cirrhosis, renal fibrosis, primary biliary cirrhosis, fatty liver disease, primary sclerosing cholangitis, restenosis, cardiac fibrosis, opthalmic scarring, fibrosclerosis, fibrotic cancers, fibroids, fibroma, fibroadenomas, fibrosarcomas, transplant arteriopathy, radiation therapy-induced fibrosis, chemotherapy-induced fibrosis, and keloid.

57. A method of inhibiting metastasis of tumor cells in a subject, the method comprising administering to said subject an effective amount of a compound of claim 1.

58. A method of inhibiting metastasis of tumor cells in a subject, the method comprising administering to said subject an effective amount of a compound of claim 39.

59. A method of inhibiting metastasis of tumor cells in a subject, the method comprising administering to said subject an effective amount of a compound of claim 40.

60. A method of treating a disease or disorder mediated by an overexpression of TGFβ, the method comprising administering to a subject in need of such treatment an effective amount of a compound of claim 1.

61. A method of treating a disease or disorder mediated by an overexpression of TGFβ, the method comprising administering to a subject in need of such treatment an effective amount of a compound of claim 39.

62. A method of treating a disease or disorder mediated by an overexpression of TGFβ, the method comprising administering to a subject in need of such treatment an effective amount of a compound of claim 40.

63. The method of claim 60, 61, or 62, said disease or disorder being selected from the group consisting of demyelination of neurons in multiple sclerosis, Alzheimer's disease, cerebral angiopathy, squamous cell carcinomas, multiple myeloma, melanoma, glioma, glioblastomas, leukemia, and carcinomas of the lung, breast, ovary, cervix, liver, biliary tract, gastrointestinal tract, pancreas, prostate, and head and neck.
